SEQUENCE LISTING (58 sequences)

SEQ ID NO: 1 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 2 - RNA, 20 nt, synthetic construct
  source 1..20: mol_type = other RNA
gtccttgccaatgaacgggg

SEQ ID NO: 3 - RNA, 20 nt, synthetic construct
  source 1..20: mol_type = other RNA
ttcaagacgggcgtgactgc

SEQ ID NO: 4 - RNA, 35 nt, synthetic construct
  source 1..35: mol_type = other RNA
ggctttatatatcttgtggaaaggacgaaacaccg

SEQ ID NO: 5 - RNA, 31 nt, synthetic construct
  source 1..31: mol_type = other RNA
gatgaatactgccatttgtctcaagatctag

SEQ ID NO: 6 - RNA, 30 nt, synthetic construct
  source 1..30: mol_type = other RNA
tggtttaacgcgtaactagatagaaccgcg

SEQ ID NO: 7 - RNA, 60 nt, synthetic construct
  source 1..60: mol_type = other RNA
aatgatacggcgaccaccgagatctacacacgtcgatttcttggctttatatatcttgtg

SEQ ID NO: 8 - RNA, 53 nt, synthetic construct
  source 1..53: mol_type = other RNA
caagcagaagacggcatacgagatcctggtagggatgaatactgccatttgtc

SEQ ID NO: 9 - RNA, 55 nt, synthetic construct
  source 1..55: mol_type = other RNA
caagcagaagacggcatacgagatagatgtgcatgtcatatgtagagaggtacct

SEQ ID NO: 10 - RNA, 44 nt, synthetic construct
  source 1..44: mol_type = other RNA
cgatttcttggctttatatatcttgtggaaaggacgaaacaccg

SEQ ID NO: 11 - RNA, 29 nt, synthetic construct
  source 1..29: mol_type = other RNA
gatcttgagacaaatggcagtattcatcc

SEQ ID NO: 12 - RNA, 29 nt, synthetic construct
  source 1..29: mol_type = other RNA
ggatgaatactgccatttgtctcaagatc

SEQ ID NO: 13 - DNA, 38 bp, synthetic construct
  source 1..38: mol_type = other DNA
cacgacgctcttccgatctaacatggaaccagcaacct

SEQ ID NO: 14 - DNA, 42 bp, synthetic construct
  source 1..42: mol_type = other DNA
gacgtgtgctcttccgatctgggagaacacagaactcaaact

SEQ ID NO: 15 - DNA, 39 bp, synthetic construct
  source 1..39: mol_type = other DNA
cacgacgctcttccgatctctgcgtgcatctttgctctg

SEQ ID NO: 16 - DNA, 40 bp, synthetic construct
  source 1..40: mol_type = other DNA
gacgtgtgctcttccgatctaactgctgcctcatcacctc

SEQ ID NO: 17 - DNA, 41 bp, synthetic construct
  source 1..41: mol_type = other DNA
cacgacgctcttccgatctcattcctccagcctcttgtatg

SEQ ID NO: 18 - DNA, 40 bp, synthetic construct
  source 1..40: mol_type = other DNA
gacgtgtgctcttccgatctcacaccatcctcggtctttg

SEQ ID NO: 19 - DNA, 38 bp, synthetic construct
  source 1..38: mol_type = other DNA
cacgacgctcttccgatctgctcgtgtacctggacgtg

SEQ ID NO: 20 - DNA, 40 bp, synthetic construct
  source 1..40: mol_type = other DNA
gacgtgtgctcttccgatctgccggaagaaatcattctga

SEQ ID NO: 21 - DNA, 60 bp, synthetic construct
  source 1..60: mol_type = other DNA
aatgatacggcgaccaccgagatctacacactgtttccctacacgacgctcttccgatct

SEQ ID NO: 22 - DNA, 66 bp, synthetic construct
  source 1..66: mol_type = other DNA
caagcagaagacggcatacgagatcctggtaggtgactggagttcagacgtgtgctcttccgatct

SEQ ID NO: 23 - DNA, 16 bp, synthetic construct
  source 1..16: mol_type = other DNA
acacacattggttccc

SEQ ID NO: 24 - DNA, 15 bp, synthetic construct
  source 1..15: mol_type = other DNA
acacacatggttccc

SEQ ID NO: 25 - DNA, 38 bp, synthetic construct
  source 1..38: mol_type = other DNA
gggaagccgctcggccgcgtggtgctggaggtgagacc

SEQ ID NO: 26 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 27 - DNA, 15 bp, synthetic construct
  source 1..15: mol_type = other DNA
aaacaggaaggagct

SEQ ID NO: 28 - DNA, 15 bp, synthetic construct
  source 1..15: mol_type = other DNA
cccttggaaggagct

SEQ ID NO: 29 - DNA, 15 bp, synthetic construct
  source 1..15: mol_type = other DNA
gcagcggaaggagct

SEQ ID NO: 30 - DNA, 15 bp, synthetic construct
  source 1..15: mol_type = other DNA
tcggtggaaggagct

SEQ ID NO: 31 - DNA, 15 bp, synthetic construct
  source 1..15: mol_type = other DNA
aaacaagccagagct

SEQ ID NO: 32 - DNA, 15 bp, synthetic construct
  source 1..15: mol_type = other DNA
aaacacccttgagct

SEQ ID NO: 33 - DNA, 15 bp, synthetic construct
  source 1..15: mol_type = other DNA
aaacaggaagttggg

SEQ ID NO: 34 - DNA, 38 bp, synthetic construct
  source 1..38: mol_type = other DNA
gggaagccgctcggccgcgtggtgctggcaacgagacc

SEQ ID NO: 35 - DNA, 38 bp, synthetic construct
  source 1..38: mol_type = other DNA
gggaagccgctcggccgcgtggtgctggtcaggagacc

SEQ ID NO: 36 - DNA, 38 bp, synthetic construct
  source 1..38: mol_type = other DNA
gggaagccgctcggccgcgtggtgctggtccagagacc

SEQ ID NO: 37 - protein, 7 aa, synthetic construct
LGRVVLE

SEQ ID NO: 38 - DNA, 25 bp, synthetic construct
  source 1..25: mol_type = other DNA
caccaagtttggggcagaggggaca

SEQ ID NO: 39 - DNA, 25 bp, synthetic construct
  source 1..25: mol_type = other DNA
gaggcagtttggggcagaggggaca

SEQ ID NO: 40 - DNA, 25 bp, synthetic construct
  source 1..25: mol_type = other DNA
gggaaagtttggggcagaggggaca

SEQ ID NO: 41 - DNA, 25 bp, synthetic construct
  source 1..25: mol_type = other DNA
tcggtagtttggggcagaggggaca

SEQ ID NO: 42 - DNA, 25 bp, synthetic construct
  source 1..25: mol_type = other DNA
caccagtgcaggggcagaggggaca

SEQ ID NO: 43 - DNA, 25 bp, synthetic construct
  source 1..25: mol_type = other DNA
caccatcggtggggcagaggggaca

SEQ ID NO: 44 - DNA, 25 bp, synthetic construct
  source 1..25: mol_type = other DNA
caccattgggggggcagaggggaca

SEQ ID NO: 45 - DNA, 25 bp, synthetic construct
  source 1..25: mol_type = other DNA
caccaagtttaacccagaggggaca

SEQ ID NO: 46 - DNA, 25 bp, synthetic construct
  source 1..25: mol_type = other DNA
caccaagtttcccttagaggggaca

SEQ ID NO: 47 - DNA, 25 bp, synthetic construct
  source 1..25: mol_type = other DNA
caccaagtttctcagagaggggaca

SEQ ID NO: 48 - DNA, 25 bp, synthetic construct
  source 1..25: mol_type = other DNA
caccaagtttggggcaacccggaca

SEQ ID NO: 49 - DNA, 25 bp, synthetic construct
  source 1..25: mol_type = other DNA
caccaagtttggggcgaggcggaca

SEQ ID NO: 50 - DNA, 25 bp, synthetic construct
  source 1..25: mol_type = other DNA
caccaagtttggggcgtgcaggaca

SEQ ID NO: 51 - DNA, 25 bp, synthetic construct
  source 1..25: mol_type = other DNA
caccaagtttggggcagaggctcag

SEQ ID NO: 52 - DNA, 25 bp, synthetic construct
  source 1..25: mol_type = other DNA
caccaagtttggggcagaggtcggt

SEQ ID NO: 53 - DNA, 25 bp, synthetic construct
  source 1..25: mol_type = other DNA
caccaagtttggggcagaggttggg

SEQ ID NO: 54 - DNA, 46 bp, synthetic construct
  source 1..46: mol_type = other DNA
tagctttgctttctcaccaagtttggggcagaggggacaaaacagg

SEQ ID NO: 55 - DNA, 30 bp, synthetic construct
  source 1..30: mol_type = other DNA
aaaaaaaaaaaaaaaaaaaaaaaaaaaaaa

SEQ ID NO: 56 - DNA, 30 bp, synthetic construct
  source 1..30: mol_type = other DNA
gggggggggggggggggggggggggggggg

SEQ ID NO: 57 - DNA, 30 bp, synthetic construct
  source 1..30: mol_type = other DNA
cccccccccccccccccccccccccccccc

SEQ ID NO: 58 - DNA, 30 bp, synthetic construct
  source 1..30: mol_type = other DNA
tttttttttttttttttttttttttttttt

(19) United States
(12) Patent Application Publication
(10) Pub. No.: US 2026/0092271 A1
(43) Pub. Date: Apr. 2, 2026

(54) METHODS AND COMPOSITIONS TO CONTROL GENE USING GENOME EDITING

(71) Applicant: The Board of Trustees of the Leland Stanford Junior University, Stanford, CA (US)

(72) Inventors: Jesse Engreitz, Millbrae, CA (US); Gabriella Martyn, Stanford, CA (US); Michael Montgomery, Stanford, CA (US); Benjamin Doughty, Stanford, CA (US); Harold Jones, Stanford, CA (US); Katherine Guo, Davis, CA (US)

(73) Assignee: The Board of Trustees of the Leland Stanford Junior University, Stanford, CA (US)

(21) Appl. No.: 19/112,198

(22) PCT Filed: Sep. 20, 2023
(86) PCT No.: PCT/US2023/074701

§ 371 Date: Mar. 14, 2025

Related U.S. Application Data
(60) Provisional application No. 63/408,256, filed on Sep. 20, 2022.

(51) Int. Cl. C12N 15/10

(52) U.S. Cl. CPC C12N 15/1082

(57) ABSTRACT

Provided herein are methods for screening edited genomic sequences for their effect on expression of a target gene, and for designing a target sequence edit for introducing to a target sequence of a nucleic acid molecule. Also provided are compositions and cells including the edited sequences, and methods for their use in preventing or treating a disease.

### CROSS-REFERENCES TO RELATED APPLICATIONS

The present application is a national stage entry under 35 U.S.C. § 371 of International Application No. PCT/US2023/074701, filed Sep. 20, 2023, which claims priority to U.S. Provisional Application No. 63/408,256, filed Sep. 20, 2022, the full disclosures of which are incorporated by reference in their entireties for all purposes.

### STATEMENT AS TO RIGHTS TO INVENTIONS MADE UNDER FEDERALLY SPONSORED RESEARCH AND DEVELOPMENT

This invention was made with Government support under contract 1656518 awarded by the National Science Foundation and under contracts HG009917 and HG011972 awarded by the National Institutes of Health. The Government has certain rights in the invention.

### SEQUENCE LISTING

The present application contains a Sequence Listing which has been submitted herewith and is hereby incorporated by reference in its entirety. Said .xml copy, created on Nov. 9, 2023, is named 079445-1400135-011310PC, and is 52,932 bytes in size.

### BACKGROUND

Genome-Wide Association Studies (GWAS) have identified >250,000 associations between genetic loci and various human diseases and traits. With ˜93% of these variants falling within noncoding regions, it is challenging to link these variants to transcription factor binding sites, target genes, and the cell types they regulate. An ideal experiment would involve editing the variants into the genome and measuring their effects on the expression of nearby genes, for example using CRISPR-Cas9 genome editing. However, such experiments for even just one variant can be time-consuming and labor-intensive, hence studying many variants in GWAS loci has not been practical using existing techniques.

Approaches are therefore needed to modify the expression of genes in certain cell types to treat disease. For example, the expression of a gene called PPIF in monocytes and macrophages is important for inflammatory bowel disease. However, it is challenging to therapeutically modulate PPIF expression in specific cell types. One solution would be to reprogram the gene regulatory sequences in the genome, which are used in nature to control gene expression in cell-type specific ways, to tune gene expression in specific cell types (e.g., to change PPIF expression in monocytes and macrophages). However, the synthetic sequences needed to change the expression of a gene in a specific way in general is not known. Designing such sequences requires new technologies to screen through many possible changes and empirically evaluate their effects on gene expression in their native genomic context. The disclosure herein provides a set of solutions to address these challenges, and offers associated and other advantages.

### BRIEF SUMMARY

The terms “invention,” “the invention,” “this invention,” and “the present invention,” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures, and each claim. Some of the exemplary embodiments of the present invention are discussed below.

In general, provided herein is a screening technology to introduce synthetic or natural edits into human gene regulatory sequences and measure their effects on gene expression at high-throughput. Also provided is a combination of this experimental screening technology with a computational approach for sequence design. The provided techniques can be used to, for example, identify specific synthetic sequences that can be introduced into regulatory sequences to change gene expression, e.g., in monocytes and T-cells. Accordingly, specific CRISPR reagents can be identified that can engineer these sequence changes efficiently into the human genome. As a result, provided methods can provide improved therapies, for example to treat inflammatory bowel disease or other diseases that depend on gene expression, e.g., PPIF expression.

In one aspect, the disclosure provides a method of screening edited genomic sequences for their effect on expression of a target gene. The method includes providing a population of cells, where the genomes of the population of cells include a target sequence. The method further includes introducing a plurality of different target sequence edits into the genomes of the population of cells. The method further includes measuring a parameter for each cell of the population of cells, where the parameter correlates with an expression level of the target gene. The method further includes separating the population of cells into a plurality of sets, i.e., bins, based on the measured parameter. The method further includes, for each combination of (a) a set of the plurality of sets and (b) a target sequence edit of the plurality of different target sequence edits, determining a count of the target sequence edit incorporated into the genomes of the cells of the population of cells in the set. The method further includes, for each target sequence edit of the plurality of different target sequence edits, based on the counts in the plurality of sets, computing an effect of the target sequence edit on the expression level of the target gene.

In another aspect, the disclosure provides a method of designing a target sequence edit for introducing to a target sequence of a nucleic acid molecule. The method includes receiving an initial target sequence edit and an initial edit site of the target sequence. The method further includes generating an initial edited sequence including the target sequence into which the initial target sequence edit is introduced at the initial edit site. The method further includes determining an initial predicted level of a molecular feature of a target gene in response to the initial edited sequence. The method further includes setting (i) an input target sequence edit equal to the initial target sequence edit, (ii) an input edit site equal to the initial edit site, and (iii) an input level equal to the initial predicted level of the molecular feature. The method further includes performing a series of operations in an iterative manner for a plurality of iterations. The series of operations includes generating a test target sequence edit and a test edit site, where either: (i) the test edit site is equal to the input edit site, and the test target sequence edit is a variant of the input target sequence edit; or (ii) the test target sequence edit is equal to the input target sequence edit, and the test edit site is one position removed from the input edit site in the target sequence. The operations further include generating a test edited sequence including the target sequence into which the test target sequence edit is introduced at the test edit site. The operations further include determining a test predicted level of the molecular feature of the target gene in response to the test edited sequence. The operations further include comparing the test predicted level and the input level. The operations further include, based on the comparing, either (i) updating (1) the input level to equal the test predicted level, (2) the input target sequence edit to equal the test target sequence edit, and (3) the input edit site to equal the test edit site; or (ii) leaving the input expression level, the input target sequence edit, and the input edit site unchanged. The method further includes, subsequent to the plurality of iterations, reporting the input target sequence edit and the input edit site.

In another aspect, the disclosure provides a composition including a plurality of template polynucleotides. The template polynucleotides each independently include a target sequence edit of the plurality of different target sequence edits of a provided method for screening edited genomic sequences for their effect on expression of a target gene.

In another aspect, the disclosure provides a population of cells. The population includes any of the template polynucleotides disclosed herein.

In another aspect, the disclosure provides a cell including a genome incorporating any of the target sequence edits disclosed herein. For example, the target sequence edit can be from a provided method for screening edited genomic sequences for their effect on expression of a target gene. Alternatively, the target sequence edit can be from a provided method for designing a target sequence edit for introducing to a target sequence of a nucleic acid molecule.

In another aspect, the disclosure provides a method of treating a subject having a disease dependent on expression of the target gene of any provided methods for screening edited genomic sequences for their effect on expression of a target gene, or of any the provided method for designing a target sequence edit for introducing to a target sequence of a nucleic acid molecule. The treatment method includes administering to the subject a therapeutically effective amount of any of the template polynucleotides disclosed herein, or a therapeutically effective amount of any of the cells disclosed herein.

These and other aspects of the disclosure are described in detail below. For example, other aspects are directed to systems, devices, and computer readable media associated with methods described herein.

Reference to the remaining portions of the specification, including the drawings and claims, will realize other features and advantages of the present disclosure. Further features and advantages of the present disclosure, as well as the structure and operation of various aspects of the present disclosure, are described in detail below with respect to the accompanying drawings. In the drawings, like reference numbers can indicate identical or functionally similar elements.

### BRIEF DESCRIPTION OF THE DRAWINGS

FIG. 1 presents an overview of a method of screening edited genomic sequences for their effect on expression of a target gene according to a provided embodiment. A plurality (e.g., 100 or more) non-coding variants targeting a cis-regulatory element (CRE) are introduced into a pool of cells using CRISPR prime-editing technology. Lentivirus is used to introduce pegRNAs into a doxycycline inducible Cas9 RT-nickase cell line. Successfully infected cells are selected via puromycin resistance and prime-editing is induced via a doxycycline treatment for 14 days. The cells are then stained for an RNA of interest and sorted into bins (e.g., sets) based on expression. The edited sites are sequenced, and the distribution of variant frequencies across the bins is determined, allows for the inference of the effect of each variant

FIG. 2 presents a schematic illustration of a pegRNA design in accordance with a provided embodiment, with the various lengths of the spacer, reverse-transcription (RT) template and primer binding site (PBS) indicated.

FIG. 3 presents a schematic illustration of a process in which a provided pegRNA ‘nicks’ the target DNA and introduces the edit via reverse transcription.

FIG. 4 presents a flowchart of a method for screening edited genomic sequences for their effect on expression of a target gene in accordance with a provided embodiment.

FIG. 5 presents a schematic illustration of principled CRISPR edit design using MCMC-style simulated annealing. The DNA sequence shown (SEQ ID NO: 24) represents a randomly initialized sequence that is iteratively mutated (e.g., to SEQ ID NO: 23) 1000× to optimize desired effects on gene expression as determined by sequence-based deep learning.

FIG. 6 presents a flowchart of s method for designing a target sequence edit for introducing to a target sequence of a nucleic acid molecule in accordance with a provided embodiment.

FIG. 7 presents a depiction of the gating strategy for an unstained FlowFISH control.

FIG. 8 presents a depiction of the gating strategy for stained FlowFISH samples.

FIG. 9 presents a histogram showing division of the stained samples into six bins, i.e., sets. based on gene expression.

FIG. 10 presents a flowchart of an exemplary step-wise process for the computational analysis of Variant-FlowFISH data.

FIG. 11 presents a schematic illustration of the prime-editing strategy used to introduce three different edits (SEQ ID NOs: 34-36) at the 5′ splice site (SEQ ID NO: 37) of the first intron of the PPIF gene (SEQ ID NO: 25) in THP-1 PE2 cells. The nucleotides to be substituted are bolded and the critical ‘GT’ nucleotides essential for splicing are underlined. The edits were introduced individually and also in a pool.

FIG. 12 presents a graph plotting the variant frequency (%) for each of the PPIF 5′ splice site edits after inducing prime-editing for 13 days through a doxycycline treatment of THP-1 PE2 cells. Shown is the mean+/−95% CI. Dots represent technical replicates from two biological replicates (n=8 dots total).

FIG. 13 presents a graph plotting the frequency of each of the variants in the PPIF 5′ splice site ‘mini-pool’ across the 6-bins (from low to high PPIF expression) after FACS sorting, as determined by sequencing the alleles in each of the bins.

FIG. 14 presents a graph plotting Variant FlowFISH data for each of the PPIF 5′ splice site edits, showing the effect of each of the edits (%) on PPIF gene expression. Shown is the mean+/−95% CI, with dots representing two technical replicates from two biological replicates (n=4 dots total).

FIG. 15 presents a graph plotting qPCR expression data for the PPIF gene, from individual PPIF 5′ splice site clonal cell lines containing each of the edits. Dots represent unedited ‘WT’ clonal cell lines (n=30), AGGT>CACC (n=20), AGGT>TCAG (n=17) and AGGT>TCCA (n=17) clonal cell lines. Shown is the mean+/−95% CI.

FIG. 16 presents a series of graphs showing results from genotyping PPIF 5′ splice site clonal populations. Clonal populations were derived for each of the PPIF 5′ splice site edits: AGGT>CAAC, TCAG, and TCCA. Homozygous edited clones were defined as containing 90-100% of the reads mapping to the intended ‘edited’ sequence; homozygous reference clones were defined as 0-10%.

FIG. 17 presents a scheme for dissecting the regulation of the PPIF locus by performing saturation mutagenesis at the PPIF enhancer, negative control region, and PPIF proximal promoter. The rs1250566 G>A SNP in the predicted PPIF enhancer is also highlighted. The chromatin accessibility (DNase-seq and ATAC-seq) across this locus is highlighted in CD14+ monocytes under various conditions.

FIG. 18 presents a list of multi-nucleotide variants (5 bp) (SEQ ID NOs: 39-53) tiled across the regions of interest (SEQ ID NO: 38) with substitutions made from a bank of 12 possible sequences.

FIG. 19 presents a list of single-nucleotide variants (SNV) (SEQ ID NOs 55-58) introduced across the region of the PPIF enhancer (SEQ ID NO: 54), centered on the rs1250566 SNP for IBD.

FIG. 20 presents a graph plotting percent total editing for the PPIF enhancer (MNV) after THP-1 PE2 cells were treated with doxycycline for 14 days.

FIG. 21 presents a frequency histogram showing the proportion of MNVs in the PPIF enhancer which are above a 0.1% threshold.

FIG. 22 presents a graph plotting a correlation of averaged PCR frequency across bio-replicates for a PPIF enhancer Variant-FlowFISH experiment in THP1 cells.

FIG. 23 presents a graph plotting a correlation of the effect size across bio-replicates for the PPIF enhancer Variant-FlowFISH experiment in THP1 cells.

FIG. 24 presents an illustration of CD14+ DNase-seq tracks and the DNase footprints across the region of the PPIF enhancer amplicon.

FIG. 25 presents an illustration of CD14+ DNase-seq tracks and the DNase footprints across a zoomed in region scrambled within the PPIF enhancer. The conservation of this region across 100 vertebrates as determined by PhastCons is also shown. The figure additionally shows the effect of each of the 5-bp multi-nucleotide variants across the PPIF enhancer, on PPIF gene expression in THP-1 cells, as determined by RNA-FlowFISH. Shown is the mean. Dots represent the average of the 4× technical and 2× biological replicates for each of the three MNV at each scramble position (n=3 dots shown). If the experiment was not able to successfully introduce a MNV into the genome, then this is indicated by # at the relevant position. The figure additionally shows results of a FIMO analysis highlighting potential candidate transcription factors which could bind to the ‘wild-type’ DNA sequence of the PPIF enhancer.

FIG. 26 presents a frequency histogram of the distance between the ‘nick and edit’ (bp) for each of the pegRNAs designed at the PPIF locus: enhancer, negative control region and the promoter.

FIG. 27 presents a frequency histogram for the reverse transcriptase (RT) template length (bp) for each of the pegRNAs designed to target the PPIF locus.

FIG. 28 presents an alignment of two scaffolds used in examples of the disclosure: Scaffold 1 RNA (SEQ ID NO: 26) and the Flip and Extension Scaffold RNA (SEQ ID NO: 1).

FIG. 29 presents a series of graphs plotting a comparison between the two scaffold RNAs used during the pegRNA design, in terms of the percentage (%) of total editing seen at the PPIF promoter, enhancer, or negative control region in THP-1 PE2 cells, after 22-50 days in doxycycline treatment (as indicated). Shown is the mean+/−95% CI and dots represent four PCR replicates.

FIG. 30 presents a graph plotting the percentage of total editing at the HEK3 locus in THP-1 PE2 cells when testing two different multiplicity of infection (MOI) amounts (0.2 MOI vs. 2 MOI). Shown are two PCR replicates from two bio-replicates.

FIG. 31 presents a graph plotting the percentage of total editing at the HEK3 locus in Jurkat cells, across time, as shown by the number of days of doxycycline treatment (which induces the PE2 editing machinery). Nine SNV edits and one MNV edit were introduced at the HEK3 locus, as shown in the legend. Coordinates for the HEK3 site are also shown.

FIG. 32 presents a graph plotting the frequency of variants (%) mapped to the PPIF enhancer. Shown is the mean at each 5-bp position, with dots representing the average variant frequency for each of the MNV. A dotted red line is shown at a frequency of 0.1%.

FIG. 33 presents a graph plotting pegRNA design features mapped to the PPIF enhancer. Shown is the ‘nick to edit’ distance for each of the pegRNAs, referring to the distance between the desired edit and where the Cas9 cuts the DNA 3 bp upstream of the spacer's PAM site. The reverse-transcriptase (RT) template length is also shown. Region 1, a region of interest within the PPIF enhancer and highlighted in FIG. 25, is also shown.

FIG. 34 presents a graph plotting the expression of candidate transcription factors in THP-1 cells. The mean transcripts per million (TMP) is shown.

FIG. 35 presents an illustration of how the ETS family of transcription factors could bind to a critical region of the PPIF enhancer (SEQ ID NO: 27). Shown is the position weight matrix from JASPAR and the effect of each of the 5-bp substitutions (SEQ ID NOs: 28-33) as determined by Variant-FlowFISH.

FIG. 36 presents a schematic overview of an experiment screening synthetic DNA insertions across multiple immune cell types and states with Variant-FlowFISH A library of 8-bp DNA sequences were inserted into the promoter of PPIF followed by Variant-FlowFISH (VFF), single molecule footprinting (SMF) and chromatin immunoprecipitation (ChIP) to assess the functional effects in THP1 monocytes.

FIG. 37 presents a histogram depicting the frequency of each insertion in the pool as a percentage of cells in the population (bottom x-axis) and equivalent number of cells assessed by VFF at a given frequency (top x-axis). The number of insertions per x-axis bin is shown on the y-axis.

FIG. 38 presents a graph plotting the power for VFF to detect a 5% effect for the 41 edits in the 8-mer pool.

FIG. 39 presents a graph plotting a correlation of observed effect sizes across technical and biological Variant-FlowFISH replicates.

FIG. 40 presents a graph plotting the effects of 8-bp insertions on PPIF expression observed by Variant-FlowFISH with benjamini-hochberg corrected p-values on the y axis. Each dot represents a different 8-bp insertion sequence and the horizontal line represents an adjusted p value of 0.05.

FIG. 41 presents a graph plotting the relative binding of NRF1 and MYC transcription factors at the wild-type PPIF promoter as determined by chromatin immunoprecipitation (ChIP) paired with quantitative PCR.

FIG. 42 presents a graph plotting the change in the binding of MYC or NRF1 relative to WT for PPIF promoter alleles containing insertions that create binding motifs for these factors. Allele-specific fold change is calculated as: [(% of reads with insertion in immunoprecipitation % of reads with insertion in whole cell genomic DNA extract) (% of unedited wild type reads in immunoprecipitation % unedited wild type reads in whole cell genomic DNA extract)]. Asterisks represent a p value ≤0.01 by one-sided t-test against an expected null fold change of 1 and “ns” is not significant.

FIG. 43 presents data showing the effects of 8-bp DNA sequence insertion library screened across three cellular conditions (rows) using Variant-FlowFISH: in THP1 monocytes, Jurkat T cells, and Jurkat T cells stimulated with immune ligands (PMA+CD3). The observed effects of each insertion (columns) on PPIF expression were calculated relative to WT cells within that condition.

FIG. 44 presents a graph plotting the effects of all insertions in the pool, grouped by condition. Outliers are labeled with the name of the TF predicted to bind the insertion sequence.

FIG. 45 presents a series of graphs plotting qPCR data for 8-bp DNA insertions on PPIF expression in clonally derived homozygous cell lines relative to WT clonally derived cell lines, and effects for the same insertions observed using Variant-FlowFISH. Each qPCR data point represents an independently generated clonal cell line and each Variant-FlowFISH data point represents a measurement from the pooled screens.

FIG. 46 presents a schematic overview of an experiment for screening edits designed using a principled in silico approach across two cell types. Enformer was used to design a library of 185 small sequence edits across 5 sites spanning the PPIF promoter. Designs included edits optimized to increase, decrease, or have no effect on gene expression.

FIG. 47 presents histograms depicting frequency of each insertion in the pool as a percentage of cells in the population (bottom x-axis) and equivalent number of cells assessed by VFF at a given frequency (top x-axis). The number of insertions per x-axis bin is shown on the y-axis and the vertical line represents an edit frequency of 0.01%.

FIG. 48 presents graphs plotting correlations of effects measured by Variant-FlowFISH in two biological replicates.

FIG. 49 presents graphs plotting effects of sequence edits on PPIF expression observed by Variant-FlowFISH with benjamini-hochberg corrected p-values on the y axis. Each dot represents a different Enformer-designed sequence in the screen and the horizontal line represents an adjusted p value of 0.05.

FIG. 50 presents graphs plotting effects of sequence edits grouped by principled design category. A subset of the edits in the screen were designed to optimally decrease, increase, or have no effect on gene expression (null) in either THP-1 or Jurkat cells.

FIG. 51 presents a graph showing the edits with the largest cell type-specific effects determined by computing the absolute difference between the effects in Jurkat and THP-1 cells and selecting those which displayed ≤10% effect on expression in one cell type. Shown are the observed Variant FlowFISH effect sizes measured in THP1 and Jurkat cells for the top 10 edits from this analysis.

FIG. 52 presents a block diagram of an exemplary measurement system in accordance with a provided embodiment.

FIG. 53 presents a block diagram of an exemplary computer system in accordance with a provided embodiment.

### DETAILED DESCRIPTION

### I. General

The present disclosure generally provides a screening technology involving introducing pools of edits into the human genome using various techniques such as prime editing, e.g., CRISPR prime editing. The technology typically further includes separating cells on the basis of some assay, such as a phenotypic assay, that correlates with the level at which a gene is expressed in a single cell. The assay can include, for example, RNA FlowFISH, immunofluorescence, growth, or other assays tailored to certain genes suitable for use in gene editing screening approaches. The frequency of edits in different pools of cells that have been separated in the course of the assay can be determined, for example via DNA sequencing. A computational approach can then be applied to infer the quantitative effect of each edit using the data about frequency of edits in each separated pool, i.e., in each set or bin. In particular examples, RNA FlowFISH can be used as the phenotypic assay, but other assays can be applied. Likewise, in particular examples, PCR and Illumina DNA sequencing can be used to read out the frequency of edits in different pools of cells, but other methods can be applied. Advantageously, the provided methods report on the effects of each individual edit on the expression of the gene corresponding to the assay.

The present disclosure also provides a computational approach for principled design and optimization of sequence edits that are tuned to have precise, cell type-specific effects on gene expression. This methodology involves initializing DNA sequence edits at a target locus, e.g., an enhancer or promoter. The initialized edits can be, for example, random edits having lengths less than or equal to 10 bp. The approach further includes predicting the effect of the initialized edit on the expression of a target gene in a specific cell type (or types) using, for example, sequence-based deep learning prediction models. The sequence edit design is then iteratively changed, for example in 1 bp increments, where each iteration is followed by effect prediction. The change to the sequence edit at each iteration is accepted of the change improves the desired outcome relative to the previous iteration. This approach to design sequence edits can beneficially be applied to, for example, maximally increase or decrease gene expression, have no change to gene expression, or maximize the effect of an edit in one cell type while minimizing the effect in another cell type. Advantageously, the approach can be adapted to optimize the design of sequence across hundreds of cellular contexts and gene regulatory layers, as the deep learning models that are relevant to this approach (e.g., Enformer, SEI) are compatible with most genomic sequence signal tracks. These include, but are not limited to, data tracks from multiple assays including chromatin immunoprecipitation with sequencing (ChIP-seq), assay for transposable chromatin with sequencing (ATAC-seq), and DNase footprinting.

The provided screening technology can be applied in many scenarios: For example, the provided methods and systems can be used to find sequence changes in coding or noncoding sequences that tune gene expression in precise ways. The provided methods and systems can additionally or alternatively be applied to find such sequences specifically in regions that contain human genetic variation associated with disease; and/or to find such sequences in the promoters of genes implicated in genetic haploinsufficiency. These applications can thus be used, for example, to restore proper levels of gene expression, such as ELN, JAG1, or NOTCH2; or to find such sequences that turn down the expression of key genes involved in immune evasion in cancer, such as CD47 or PDL1/CD247. Beneficially, the provided methods and materials are also generally suitable for use in other applications in which precise changes in gene expression are desired as therapeutics.

As one example, the processes disclosed herein can be used to identify specific synthetic sequences that modulate PPIF expression in monocytes and T-cells. These specific synthetic sequence changes can be introduced into specific locations in the human genome to obtain precise changes in gene expression in these cell types. Further, because of the function of PPIF in inflammatory bowel disease (Nasser et al., Nature 593, (2021): 238), the provided sequence edits identified using the disclosed approaches can be used to tune monocyte function and treat inflammatory bowel disease. These synthetic sequences can be introduced into the human genome using any number of approaches. For example, CRISPR prime editing reagents can be introduced to a subject in need of treatment. Alternatively or additionally, edits can be introduced with other approaches such a CRISPR cutting with a template for homology directed repair or other techniques known in the field of genome editing.

While the provided genome editing materials can be prime editing reagents, e.g., CRISPR reagents for introducing targeted changes based on CRISPR prime editing, other approaches are also suitable for use with the provided materials and methods. In some examples, a specific version of CRISPR prime editing (PE2) is used to introduce sequence changes, for example with a specific pegRNA sequence and nCas9-MMLV construct. Advantageously, however, other types of prime editing strategies (involving fusing a specific DNA recognition domain, a reverse transcriptase, and a template RNA for reverse transcription) can be used to introduce the provided sequence changes for therapeutic benefit.

### II. Definitions

As used herein, the terms “polynucleotide” and “nucleic acid” refer to deoxyribonucleic acids (DNA) or ribonucleic acids (RNA) and polymers thereof in either single- or double-stranded form. Unless specifically limited, the term encompasses nucleic acids containing known nucleotide analogs or modified backbone residues or linkages, which are synthetic, naturally occurring, and non-naturally occurring, that have similar binding properties as the reference nucleic acid and are metabolized in a manner similar to reference nucleotides. Examples of such analogs include, without limitation, phosphorothioates, phosphoramidates, methyl phosphonates, chiral-methyl phosphonates, 2-O-methyl ribonucleotides, and peptide nucleic acids (PNAs). Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences, as well as the sequence explicitly indicated. Specifically, degenerate codon substitutions may be achieved by generating sequences in which the third position of one or more selected (or all) codons is substituted with mixed-base and/or deoxyinosine. Polynucleotide sequences, when provided, are listed in the 5′ to 3′ direction, unless stated otherwise. Nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

The abbreviation “bp” refers to base pairs. In some instances, “bp” may be used to denote a length of a DNA fragment, even though the DNA fragment may be single stranded and does not include a base pair. In the context of single-stranded DNA, “bp” may be interpreted as providing the length in nucleotides.

As used herein, the term “gene” refers to a segment of DNA involved in producing a polypeptide chain. A gene may include regions preceding and/or following the coding region (e.g., a leader and/or trailer) as well as intervening sequences (e.g., introns) between individual coding segments (exons).

As used herein, the term “sequence read” refers to a string of nucleotides obtained from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes as may be used in microarrays, or amplification techniques, such as the polymerase chain reaction (PCR) or linear amplification using a single primer or isothermal amplification. Example sequencing techniques include massively parallel sequencing, targeted sequencing, Sanger sequencing, sequencing by ligation, ion semiconductor sequencing, and single molecule sequencing (e.g., using a nanopore, or single-molecule real-time sequencing (e.g., from Pacific Biosciences)). Such sequencing can be random sequencing or targeted sequencing (e.g., by using capture probes hybridizing to specific regions or by amplifying certain region, both of which enrich such regions). Example PCR techniques include real-time PCR and digital PCR (e.g., droplet digital PCR). As part of an analysis of a biological sample, a statistically significant number of sequence reads can be analyzed, e.g., at least 1,000 sequence reads can be analyzed. As other examples, at least 5,000, 10,000 or 50,000 or 100,000 or 500,000 or 1,000,000 or 5,000,000 sequence reads, or more, can be analyzed.

As used herein, the term “site” (also called a “genomic site”) corresponds to a single site, which may be a single base position or a group of correlated base positions, e.g., a CpG site, TSS site, DNase hypersensitivity site, or larger group of correlated base positions.

As used herein, the terms “identity,” “substantial identity,” “similarity,” “substantial similarity,” “homology” and the related terms and expressions used in the context of describing nucleic acid or amino acid sequences refer to a sequence that has at least 60% sequence identity to a reference sequence. Examples include at least: 60%, 65%, 70%, 75%, 80%, 85%, 86%, 87%, 88%, 89%, 90%, 91%, 92%, 93%, 94%, 95%, 96%, 97%, 98%, or 99%, sequence identity, as compared to a reference sequence using the programs for comparison of nucleic acid or amino acid sequences, such as BLAST using standard parameters. For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default (standard) program parameters can be used, or alternative parameters can be designated. The sequence comparison algorithm then calculates the percent sequence identities for the test sequences relative to the reference sequence, based on the program parameters. A “comparison window” includes reference to a segment of any one of the number of contiguous positions (from 20 to 600, usually about 50 to about 200, more commonly about 100 to about 150), in which a sequence may be compared to a reference sequence of the same number of contiguous positions after the two sequences are optimally aligned. Methods of alignment of sequences for comparison are well-known. Optimal alignment of sequences for comparison may be conducted, for example, by the local homology algorithm of Smith and Waterman (Smith and Waterman “Identification of common molecular subsequences.” J Mol Biol. 147(1):195-7 (1981)) by the homology alignment algorithm of Needleman and Wunsch (Needleman and Wunsch “A general method applicable to the search for similarities in the amino acid sequence of two proteins.” J Mol Biol. 48(3):443-53 (1970)), by the search for similarity method of Pearson and Lipman (Pearson and Lipman “Improved tools for biological sequence comparison.” Proc Natl Acad Sci USA. 85(8):2444-8 (1988)), by computerized implementations of these algorithms (for example, BLAST), or by manual alignment and visual inspection.

Algorithms that are suitable for determining percent sequence identity and sequence similarity include BLAST and BLAST 2.0 algorithms, which are described in Altschul et al. “Basic local alignment search tool.” J. Mol. Biol. 215:403-410 (1990), and Altschul et al., “Gapped BLAST and PSI-BLAST: a new generation of protein database search programs.” Nucleic Acids Res. 25:3389-3402 (1997), respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (Henikoff and Henikoff, “Amino acid substitution matrices from protein blocks.” Proc. Natl. Acad. Sci. USA 89:10915-10919 (1989)). The BLAST algorithm also performs a statistical analysis of the similarity between two sequences (Karlin and Altschul “Applications and statistics for multiple high-scoring segments in molecular sequences.” Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

BLAST nucleotide searches may be performed with the BLASTN program (nucleotide query searched against nucleotide sequences) to obtain nucleotide sequences homologous to nucleic acid molecules of this disclosure, or with the BLASTX program (translated nucleotide query searched against protein sequences) to obtain protein sequences homologous to nucleic acid molecules of the invention. BLAST protein searches may be performed with the BLASTP program (protein query searched against protein sequences) to obtain amino acid sequences homologous to protein molecules of this disclosure, or with the TBLASTN program (protein query searched against translated nucleotide sequences) to obtain nucleotide sequences homologous to protein molecules of this disclosure. To obtain gapped alignments for comparison purposes, Gapped BLAST (in BLAST 2.0) may be utilized as described in Altschul et al. (1997) Nucleic Acids Res. 25:3389. Alternatively, PSI-Blast may be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) may be used.

As used herein, the term “operably linked” refers to a functional linkage between a nucleic acid expression control sequence (such as a promoter, enhancer, or array of transcription factor binding sites) and a second nucleic acid sequence, wherein the expression control sequence directs transcription of the nucleic acid corresponding to the second sequence.

The term “introducing,” as used in the context of a polynucleotide described herein, refers to presenting a nucleic acid sequence to a host cell in such a manner that the sequence gains access to the interior of the cell. Methods for introducing nucleic acid sequences into cells are known in the art and include, but are not limited to, stable transformation methods, transient transformation methods, and virus-mediated methods. “Stable transformation” is intended to mean that the nucleotide construct introduced into a host cell integrates into the genome of the host cell or plant and is capable of being inherited by the progeny thereof. “Transient transformation” is intended to mean that a polynucleotide is introduced into the host cell and does not integrate into the genome of the host cell, or that a polypeptide is introduced into a host cell.

As used herein, the term “subject” refers to a vertebrate, and preferably to a mammal. Mammalian subjects for which the provided composition is suitable include, but are not limited to, mice, rats, simians, humans, farm animals, sport animals, and pets. In some embodiments, the subject is human. In some embodiments, the subject is male. In some embodiments, the subject is female. In some embodiments, the subject is an adult. In some embodiments, the subject is an adolescent. In some embodiments, the subject is a child. In some embodiments, the subject is above 10 years of age, e.g., above 20 years of age, above 30 years of age, above 40 years of age, above 50 years of age, above 60 years of age, above 70 years of age, or above 80 years of age. In some embodiments, the subject is less than 80 years of age, e.g., less than 70 years of age, less than 60 years of age, less than 50 years of age, less than 40 years of age, less than 30 years of age, less than 20 years of age, or less than 10 years of age.

As used herein, the term “administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

As used here, the terms “treat”, “treating” and “treatment” refer to a procedure resulting in any indicia of success in the elimination or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom. The treatment or amelioration of symptoms can be based on any objective or subjective parameter; including, e.g., the result of a physical examination.

As used herein, the terms “pharmaceutically acceptable excipient” and “pharmaceutically acceptable carrier” refer to a substance that aids the administration of an active agent to and absorption by a subject and may be included in the compositions of the present disclosure without causing a significant adverse toxicological effect on the subject. Non-limiting examples of pharmaceutically acceptable excipients and carriers include water, NaCl, normal saline solutions, normal sucrose, normal glucose, binders, fillers, disintegrants, lubricants, coatings, and the like. One of skill in the art will recognize that other pharmaceutically acceptable excipients and carriers are useful in the present disclosure.

As used herein, the term “therapeutically effective amount” refers to an amount or dose of a compound, composition, or formulation that produces therapeutic effects for which it is administered. The exact amount or dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the terms “including,” “comprising,” “having,” “containing,” and variations thereof, are inclusive and open-ended and do not exclude additional, unrecited elements or method steps beyond those explicitly recited. As used herein, the phrase “consisting of” is closed and excludes any element, step, or ingredient not explicitly specified. As used herein, the phrase “consisting essentially of” limits the scope of the described feature to the specified materials or steps and those that do not materially affect the basic and novel characteristics of the disclosed feature.

As used herein, the singular forms “a,” “an,” and “the” include plural referents unless the content clearly dictates otherwise. Thus, for example, reference to “a Toll-like receptor agonist” optionally includes a combination of two or more Toll-like receptor agonists, and the like.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

Standard abbreviations may be used, e.g., bp, base pair(s); kb, kilobase(s); pi, picoliter(s); s or sec, second(s); min, minute(s); h or hr, hour(s); aa, amino acid(s); nt, nucleotide(s); and the like.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described.

### III. Methods for Screening Edited Sequences

In one aspect of the present disclosure, a method is provided for screening edited genomic sequences for their effect on expression of a target gene. The method, named Variant FlowFISH in some embodiments, advantageously provides a high-throughput means for introducing a large number of noncoding variants into a population of cells using pooled prime editing technology, and measuring the effect of each of the variants on the expression of a gene of interest. Prime editing is a precise genome editing technology, which introduces undesired mutations (such as indels) at a very low frequency compared to other editing methods, e.g., CRISPR-Cas9 editing methods. The examples described herein demonstrate that the provided method is advantageously precise and sensitive, introducing up to 185 variants or more within a single pooled experiment, with a total editing rate up to 40% or higher. Furthermore, the provided method overcomes limitations of previous experimental approaches by reading out the effects in single cells, avoiding the need to derive and genotype clonal cell lines. Each variant edit in the genome can be present in >1000 single cells, thereby providing quantitative measurements with power to detect small effects on gene expression, e.g., as low as 5% or lower. This transformative technology can therefore be useful for characterizing disease variants, dissecting the regulatory logic of enhancers and promoters, and reprogramming DNA sequences to modulate expression in endogenous regulatory contexts.

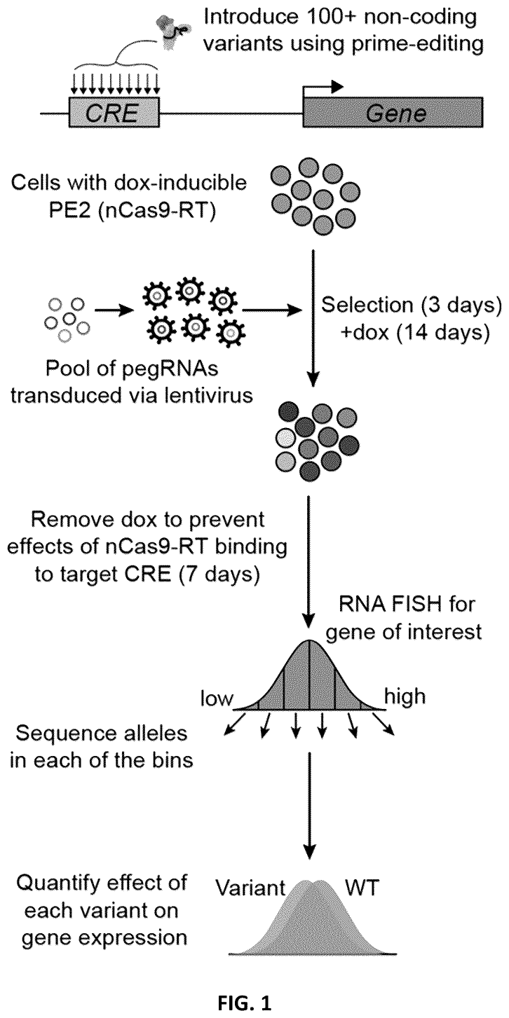

In some embodiments, the provided screening method combines prime editing and RNA FlowFISH assays (FIG. 1). Briefly, in this approach, prime editing is first applied to introduce up to 100+ variants in a single pooled experiment. For example, both the PE2 and PE3 systems (Anzalone et al., Nature 576, (2019): 149) and epegRNA (Nelson et al., Nat. Biotechnol. 40, (2022): 402) are suitable for introducing edits during this process. Importantly, prime editing avoids the introduction of large insertions or deletions common in homology-directed repair approaches, which can confound effect size measurements. Next, this population of edited cells is used for performing RNA FlowFISH for the gene of interest, sorting the cells into, for example, 4 or 6 bins or sets based on their expression levels of this gene. The sorting can be, for example, via fluorescent activated cell sorting (FACS). In some embodiments, the edited site in each of the bins is PCR-amplified and sequenced to infer the effect of each variant on gene expression based on the frequency distribution of the variant across the bins.

This provided approach thus enables the characterization of 100 or more variants within a single pooled experiment by determining the effects of the variants on the expression of a gene of interest over the span of a time period of 3-4 weeks or less. Furthermore, this approach beneficially accounts for inherent inefficiencies or inaccuracies in existing genome editing methods. For example, even if editing is inefficient (e.g. 0.1% frequency) or introduces multiple edits or indels, the provided approach allows for measuring the effect of each allele through pooled sequencing and analysis of allele frequencies in a population of cells.

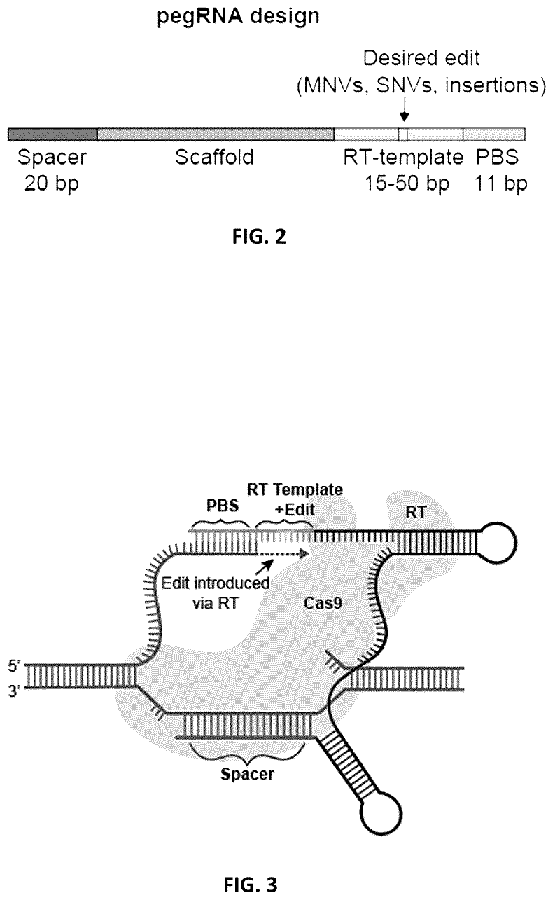

In some embodiments, the targeted genome edits, i.e., target sequence edits, are introduced using prime-editing guide RNA (pegRNA) molecules. A schematic of the structure of a pegRNA suitable for use with the provided method, and the different components of the pegRNA molecule, is shown in FIGS. 2 and 3.

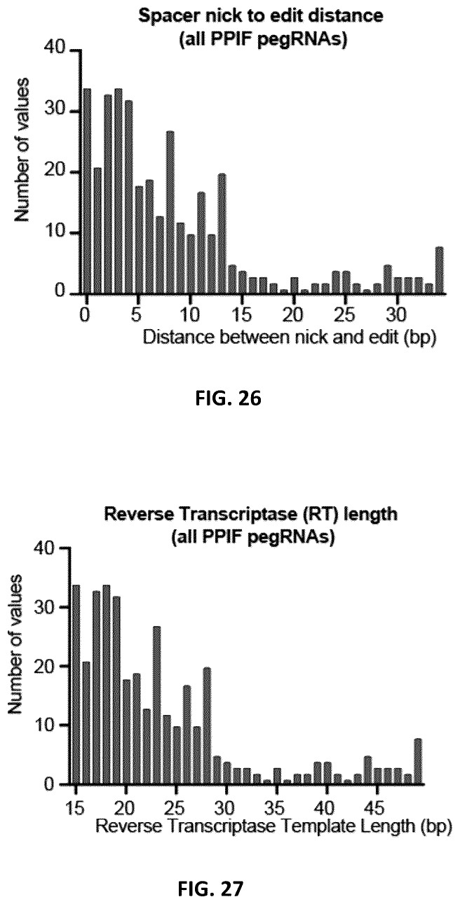

The pegRNA generally includes a spacer region. In certain examples, the spacer is selected based on the specificity score and distance of the edit or variant to a ‘nick’ of the target sequence at which the edit is introduced. As a general rule, the closer the spacer to the edit, the more likely that the edit will be introduced at a higher efficiency. Spacer selection is limited by the availability of PAM sequences and the spacer is always 5′ to the edit. In some embodiments, the spacer is up to 50 bp away from the desired edit. In certain examples, the spacer is no more than 35 bp away from the edit (FIG. 26). For example, the spacer length can be set at 20 bp (FIG. 2).

In some embodiments, each of the pegRNA molecules includes a gRNA spacer that is no more than 50 bp away from the target sequence edit. The spacer length can be, for example, between 0 bp and 50 bp, e.g., between 0 bp and 10 bp, between 1 bp and 15 bp, between 2 bp and 23 bp, between 3 bp and 35 bp, or between 5 bp and 50 bp. In terms of upper limits, the spacer length can be, for example, not more than 50 bp, e.g., no more than 35 bp, no more than 23 bp, no more than 15 bp, no more than 10 bp, no more than 7 bp, no more than 5 bp, no more than 3 bp, no more than 2 bp, or no more than 1 bp. In terms of lower limits, the spacer length can be at least 1 bp, e.g., at least 2 bp, at least 3 bp, at least 5 bp, at least 7 bp, at least 10 bp, at least 15 bp, at least 23 bp, or at least 35 bp.

The pegRNA also generally includes a scaffold region. In some embodiments, the scaffold region has at least 85% sequence identity with the scaffold sequence GTTTAAGAGCTATGCTGGAAACAGCATAGCAAGTTTAAATAAGGCTAGTCCGTTA TCAACTTGAAAAAGTGGCACCGAGTCGGTGC (SEQ ID NO: 1). The scaffold region can have, for example, at least 87%, at least 89%, at least 91%, at least 93%, at least 95%, at least 96%, at least 97%, at least 98%, at least 99%, or 100% sequence identity with the scaffold sequence GTTTAAGAGCTATGCTGGAAACAGCATAGCAAGTTTAAATAAGGCTAGTCCGTTA TCAACTTGAAAAAGTGGCACCGAGTCGGTGC (SEQ ID NO: 1).

The pegRNA also generally includes a primer binding site (PBS). The length of the PBS can be, for example, between 6 bp and 12 bp, between 7 bp and 13 bp, between 8 bp and 14 bp, between 9 bp and 15 bp, or between 10 bp and 16 bp. In terms of upper limits, the PBS length can be, for example, no more than 16 bp, e.g., no more than 15 bp, no more than 14 bp, no more than 13 bp, no more than 12 bp, no more than 11 bp, no more than 10 bp, no more than 9 bp, no more than 8 bp, no more than 7 bp, or no more than 6 bp. In terms of lower limits, the PBS length can be, for example, at least 6 bp, e.g., at least 7 bp, at least 8 bp, at least 9 bp, at least 10 bp, at least 11 bp, at least 12 bp, at least 13 bp, at least 14 bp, at least 15 bp, or at least 16 bp. In certain examples, the PBS length is set to 11 bp. (FIG. 2).

The pegRNA also generally includes a reverse transcription (RT) template. The length of the RT template can be, for example, between 10 bp and 60 bp, e.g., between 10 bp and 40 bp, between 15 bp and 45 bp, between 20 bp and 50 bp, between 25 bp and 55 bp, or between 30 bp and 60 bp. In terms of upper limits, the RT template length can be, for example, no more than 60 bp, e.g., no more than 55 bp, no more than 50 bp, no more than 45 bp, no more than 40 bp, no more than 35 bp, no more than 30 bp, no more than 25 bp, no more than 20 bp, no more than 15 bp, or no more than 10 bp. In terms of lower limits, the RT template length can be at least 10 bp, e.g., at least 15 bp, at least 20 bp, at least 25 bp, at least 30 bp, at least 35 bp, at least 40 bp, at least 45 bp, at least 50 bp, at least 55 bp, or at least 60 bp. In certain examples, the RT template has a length between 15 bp and 50 bp (FIG. 2).

In some embodiments, the RT template extends between 6 bp and 16 bp upstream of the target sequence edit. For example, the RT template can extend upstream of the target sequence edit between 6 bp and 12 bp, between 7 bp and 13 bp, between 8 bp and 14 bp, between 9 bp and 15 bp, or between 10 bp and 16 bp. In terms of upper limits, the RT template can extend upstream of the target sequence edit, for example, no more than 16 bp, e.g., no more than 15 bp, no more than 14 bp, no more than 13 bp, no more than 12 bp, no more than 11 bp, no more than 10 bp, no more than 9 bp, no more than 8 bp, no more than 7 bp, or no more than 6 bp. In terms of lower limits, the RT template can extend upstream of the target sequence edit, for example, at least 6 bp, e.g., at least 7 bp, at least 8 bp, at least 9 bp, at least 10 bp, at least 11 bp, at least 12 bp, at least 13 bp, at least 14 bp, at least 15 bp, or at least 16 bp.

In some embodiments the pegRNA includes a 3′ modification configured or selected to increase editing. For example, the pegRNAs can be modified with a 3′ modification to the RNA transcript (termed engineered pegRNA or epegRNA), where the 3′ modification has at least 86% sequence identity with the 37-bp trimmed evopreRQ (tevopreQ1) pseudoknot sequence (Nelson et al., Nat. Biotechnol. 40, (2022): 402). The 3′ modification can have, for example, at least 87%, at least 89%, at least 91%, at least 93%, at least 95%, at least 96%, at least 97%, at least 98%, at least 99%, or 100% sequence identity with the 37-bp trimmed evopreRQ (tevopreQ1) pseudoknot sequence.

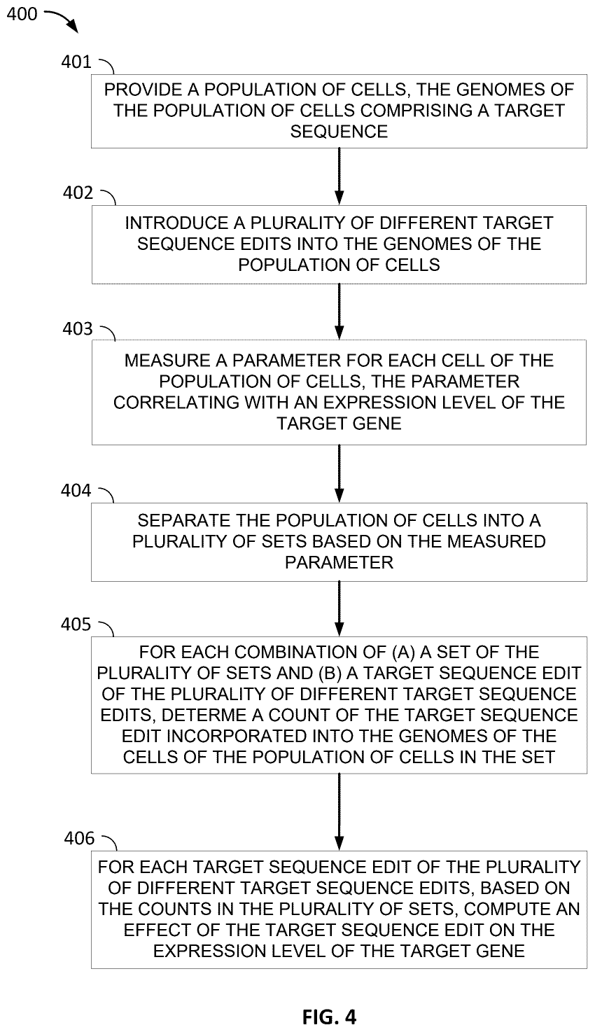

FIG. 4 presents a flowchart of a provided method 400 for screening edited genomic sequences for their effect on expression of a target gene. The edits can include, for example, mutagenesis tiling edits, single nucleotide variants (SNVs), and/or insertions sequences. Various examples of method 400 are described in Section III and Examples 1-4. Method 400 can be performed partially or entirely using a computer system.

At block 401 of method 400, a population of cells is provided, where the genomes of the population of cells include a target sequence. In some embodiments, the population of cells contains a fusion protein, where the fusion protein includes a polymerase and a DNA targeting protein. The DNA targeting protein can have a nickase activity, and/or can bind to the target sequence. In some embodiments, the population of cells includes cells of an inducible Cas9 RT-nickase cell line.

In some embodiments, the target sequence includes a cis-regulatory element (CRE) operably linked to the target gene. The target sequence can be, for example, a sequence or subsequence of a promoter. The target sequence can be a sequence or subsequence of an enhancer. The target sequence can be a sequence or a subsequence of a transcription factor binding region.

In some embodiments, the target sequence is identified or selected by selecting genes known to be involved in a disease through genetic evidence (e.g., coding variants or deletions associated with common or rare disease), or using predictive models (such as ABC (Fulco et al., Nat. Genetics 51, (2019): 1664) or other approaches) to determine the regulatory elements that would control the gene of interest. The target sequence can be identified by selecting genes known to be involved in a disease through genetic evidence (e.g., coding variants or deletions associated with common or rare disease), or using CRISPRi-FlowFISH (see International Patent Application Publication No. WO2018/064208A1) to identify the regulatory elements that control the gene of interest. The target sequence can be identified by selecting genes known to be involved in haploinsufficiencies (e.g., ELN for Williams Syndrome, JAG1 or NOTCH2 for Alagille Syndrome), where it is expected that turning the expression of a gene up by 2-fold would be therapeutic, and selecting the promoters, enhancers, or splice sites around that gene to conduct saturation mutagenesis or design other edits in order to increase the expression of that gene The target sequence can be identified or selected by computationally identifying candidate regulatory elements (by ATAC-seq, ABC, or other methods) that overlap variants associated with human diseases through genome-wide association studies. The target sequence can be identified or selected by conducting high-throughput CRISPR screens to find elements that modulate the expression of a cellular phenotype of interest, such as differentiation of red blood cells, proliferation of T cells, production of elastin fibers by endothelial cells, etc.

At block 402 of method 400, a plurality of different target sequence edits is introduced into the genomes of the population of cells. At least a portion of the plurality of different target sequence edits can each independently include, for example, one or more substitutions relative to the target sequence. At least a portion of the plurality of different target sequence edits can each independently include one or more insertions relative to the target sequence. At least a portion of the plurality of different target sequence edits can each independently include one or more deletions relative to the target sequence.

The target sequence edits can be designed by, for example, choosing transcription factor motifs that are known and/or predicted to increase or decrease gene expression, e.g. using existing tools. The target sequence edits can be designed by using predictive models (e.g. ENFORMER, BPNet, DeepSTARR, SEI) to predict specific synthetic sequences that will have desired effects on gene expression (Avsec et al., Nat. Methods 18, (2021): 1196). The target sequence edits can be designed by pre-screening for sequence edits that would be likely to up-regulate or down-regulate gene expression by specific amounts using plasmid-based reporter assays or other reporter assays. The target sequence edits can be designed by changing specific noncoding variants that occur in a given person back to the sequence present in the reference genome sequence, in order to restore expression to its normal level. The target sequence edits can be selected by designing insertions (e.g., insertions that are 1-50 nucleotides in length) of specific or random sequences, where the maximum length is specified as sequences that can be PCR-amplified from the genome. The target sequence edits can be selected by designing sequence deletions (e.g., deletions that are 1-50 nucleotides in length). The target sequence edits can be selected by designing sequence inversions that might change the direction of a regulatory sequence or CTCF site (e.g., inversions that are 2-50 nucleotides in length), where the maximum length is specified as sequences that can be PCR-amplified from the genome. The target sequence edits can be selected by designing edits to existing sequence motifs to change binding affinity for regulatory factors including transcriptional activators and repressors. These edits could potentially include (1) changing nucleotides within an existing transcription factor binding site motif and (2) modifying the sequences that flank a transcription factor binding site motif.

In some embodiments, the plurality of different target sequence edits includes between 3 and 1000 different target sequence edits. The number of different target sequence edits can be, for example, between 3 and 100, between 10 and 160, between 15 and 250, between 25 and 400, between 40 and 630, or between 63 and 1000. In terms of upper limits, the number of different target sequence edits can be, for example, no more than 1000, e.g., no more than 630, no more than 400, no more than 250, no more than 160, no more than 100, no more than 63, no more than 40, no more than 25, no more than 15, no more than 10, or no more than 3. In terms of lower limits, the number of different target sequence edits can be, for example, at least 3, e.g., at least 10, at least 15, at least 25, at least 40, at least 63, at least 100, at least 160, at least 250, at least 400, at least 630, or at least 1000.

The introducing of the plurality of different target sequence edits can use, for example, any approach delivering a polymerase (e.g., MMLV or other enzymes) to a particular location in the genome by covalent or inducible fusion to a sequence-specific DNA targeting protein with DNA-nicking capabilities (e.g., CRISPR/Cas9, Cas12a, TALENs, Zinc finger nucleases); and simultaneously delivering into cells a template nucleotide molecule (e.g., pegRNA, separate circular RNA, or other RNA template delivery approaches) that can be primed by the nicked genomic DNA and copied into the DNA sequence by the polymerase. Delivery approaches suitable for use with the provided method include, for example, those described in International Patent Application Publication No. WO 2020/191241, the full disclosure of which is incorporated herein by reference in its entirety for all purposes. Other suitable approaches include those for delivering a designed template using sequence-targeted nickases or nucleases, and using DNA or RNA ligation to install the designed template at the target site. These approaches include those described in US Patent Application Publication No. US 2023/0151353, the full disclosure of which is incorporated herein by reference in its entirety for all purposes.

In some embodiments, the introducing of the plurality of different target sequence edits includes introducing to the population of cells a plurality of template polynucleotides. The template polynucleotides can each independently include a target sequence edit of the plurality of different target sequence edits. In some embodiments, the plurality of template polynucleotides are included in a plurality of pegRNA molecules. The pegRNA molecules can have, for example, any of the characteristics described in Section III. In some embodiments, in at least a portion of the population of cells, a template polynucleotide of the plurality of template polynucleotides is primed by a target sequence nicked by a DNA targeting protein of a fusion protein expressed by the cells. Subsequently, the template polynucleotide can be copied into the genome by the polymerase of the fusion protein. In some embodiments, the method includes introducing to the population of cells an inducer of expression of the fusion protein.

In some embodiments, the introducing of the plurality of different target sequence edits includes use of a lentivirus, a transposase, or a recombinase. In certain examples, the introducing includes transducing the population of cells with lentivirus.

At block 403 of method 400, a parameter for cell of the population of cells is measured, where the parameter correlates with an expression level of the target gene. In some embodiments, the measuring of the parameter includes using a phenotypic assay. The phenotypic assay can include, for example, a fluorescence-in-situ-hybridization (FISH) assay, The phenotypic assay can include a fluorescence assay. The phenotypic assay can include an immunofluorescence assay. The phenotypic assay can include a growth assay. In some embodiments, the measuring of the parameter includes antibody staining for the protein from the target gene of interest (e.g. IL2RA) and sorting via fluorescence activated cell sorting (FACS) for that protein of interest.

At block 404 of method 400, the population of cells is separated into a plurality of sets, e.g., bins, based on the measured parameter. In some embodiments, the separating of the population of cells includes FACS. In some embodiments, the population of cells is separated into between 3 and 100 sets. The number of sets can be, for example, between 3 and 20, between 4 and 30, between 6 and 45, between 9 and 70, or between 14 and 100. In terms of upper limits, the number of sets can be, for example, no more than 100, e.g., no more than 70, no more than 45, no more than 45, no more than 30, no more than 20, no more than 14, no more than 9, no more than 6, no more than 4, or no more than 3. In terms of lower limits, the number of sets can be, for example, at least 3, e.g., at least 4, at least 6, at least 9, at least 14, at least 20, at least 30, at least 45, at least 70, or at least 100.

At block 405 of method 400, for each combination of (a) a set of the plurality of sets and (b) a target sequence edit of the plurality of different target sequence edits, a count of the target sequence edit incorporated into the genomes of the cells of the population of cells in the set is determined.

In some embodiments, the determining of the counts of the plurality of different target sequence edits includes receiving a plurality of sequence reads. The determining of the count of the plurality of different target sequence edits can include sequencing a target region of the genomes of the population of cells, where the target region includes the target sequence prior to the introducing of the plurality of different target sequence edits into the population of cells.

The determining of the counts can further include, for each of the plurality of sequence reads, determining if the sequence read belongs to an aligning set consisting of the sequence reads of the plurality of sequence reads that align to a reference amplicon, where the reference amplicon includes the target sequence. The determining of the counts can further include, for each variant sequence of a set of variant sequences, determining a count of the sequence reads of the aligning set that include the variant sequence, where each variant sequence of the set of variant sequences independently includes the target sequence modified according to a target sequence edit of the plurality of different target sequence edits. The determining of the counts can further include determining a count of the sequence reads of the aligning set that do not match any variant sequence of the set of variant sequences, and that differ from the target sequence by no more than a threshold percentage of base pairs.

The threshold percentage of base pairs used in determining the counts can be, for example, between 1% and 15%, e.g., between 1% and 6%, between 2% and 8%, between 3% and 10%, between 4% and 12%, between 5% and 15%. In terms of upper limits, the threshold percentage can be, for example, no more than 15%, e.g., no more than 12%, no more than 10%, no more than 8%, no more than 6%, no more than 5%, no more than 4%, no more than 3%, no more than 2%, or no more than 1%. In terms of lower limits, the threshold percentage can be, for example, at least 1%, e.g., at least 2%, at least 3%, at least 4%, at least 5%, at least 6%, at least 8%, at least 10%, at least 12%, or at least 15%.

At block 406 of method 400, for each target sequence edit of the plurality of different target sequence edits, based on the counts in the plurality of sets, an effect of the target sequence edit on the expression level of the target gene is computed. In some embodiments, the computing of the effect of the target sequence edit includes, for each target sequence edit of the plurality of different target sequence edits, fitting the counts in the plurality of sets to a log-normal distribution. The computing of the effect can further include calculating a percent difference between (a) the log-normal distribution for the target sequence edit, and (b) a log-normal distribution derived by fitting counts of the target sequence in the plurality of sets.

Desirable qualities of DNA sequence edits that could be discovered and measured through the provided screening methods, and that would be helpful for therapeutic purposes include, for example, the ability for a DNA sequence edit to tune gene expression to precise levels, the ability for a DNA sequence edit to modulate gene expression in one cell type but not in another, and/or the ability for a DNA sequence edit to modulate gene expression in a given cell type only in the context of a particular cellular stimulus (such as IL1B activation of monocytes, activation of T cells, smooth muscle cells in serum+ vs serum− cell states).

Variations on the provided method can also enable screening of the effects of larger sequence changes, such as regulatory sequences up to 10 kb. Edits are introduced using a two-step process similar to described by Twin Prime Editing or PASTE, involving first installing a recognition site for a serine recombinase, and second using a serine recombinase to insert a desired sequence of 100-10 kb into that site. Flow sorting or other phenotypic selection can be conducted as in the method described in Section III. The frequency of edits can be read out through one or more of the following: PCR using primers targeting either side of the insertion site (in the cases where the amplicon size is amenable); one primer outside the insertion site and one primer inside the insertion; or both primers inside the insertion, either to read out the full inserted sequence or the sequence of a designed barcode that is unique to the inserted sequence. The effects of edits can be inferred as in the method described in Section III.

### IV. Methods for Designing Sequence Edits

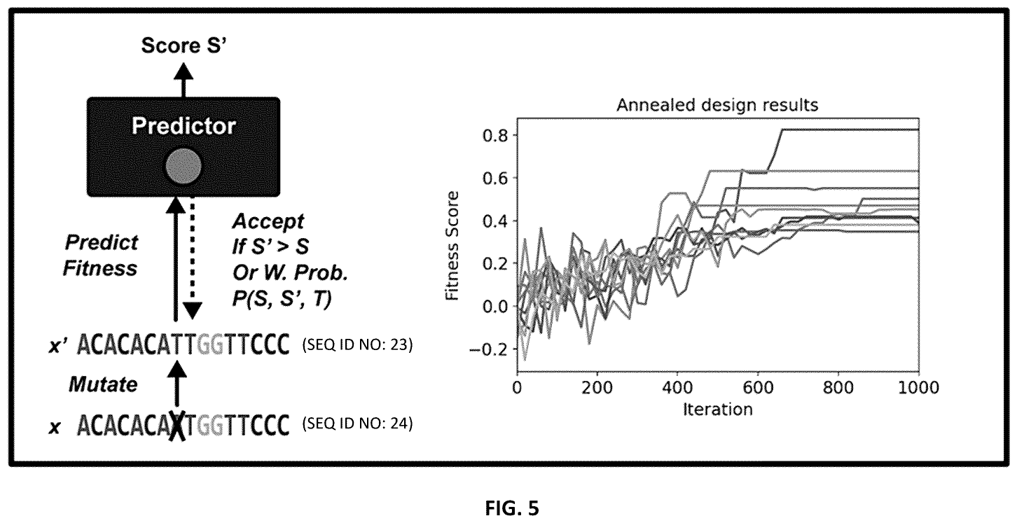

In another aspect of the present disclosure, a method is provided for the design and optimization of sequence edits in silico. The sequence edits designed and optimized using the provided method can be particularly useful in combination with the edit screening methods described in Section III. For example, the in silico sequence edit optimization methods of Section II can provide a source of sequence edits to be tested using the screening methods of Section III. In some embodiments, using the CAGE prediction tracks for THP1 and Jurkat cell types from the Enformer sequence-based deep learning model (Avsec et al., Nat. Methods 18, (2021): 1196), MCMC-style simulated annealing is applied, e.g., to optimize short sequence edits for five potential CRISPR-targetable loci in the PPIF promoter. In one example, starting from a randomly initialized sequence insertion (<=10 bp) at the targetable loci, 1000 randomly sampled alterations to the sequence edit were performed (involving either: (a) inserting one new nucleotide, (b) deleting one nucleotide, (c) shifting the insertion site by one position, or (d) substituting one existing nucleotide for another letter) (FIG. 5). The total edited sequence was restricted from growing beyond 10 bp. Edit alterations that increased the objective function sought to be optimized were accepted. Importantly, with a probability that decreases smoothly and monotonically as a function of the number of alterations lapsed (until the fixed budget of 1000 alterations has been made), ‘bad’ alterations that decrease the objective function value were accepted. By annealing slowly, short sequence edits were found that reach near-global optimal impact on promoter function as predicted by Enformer.

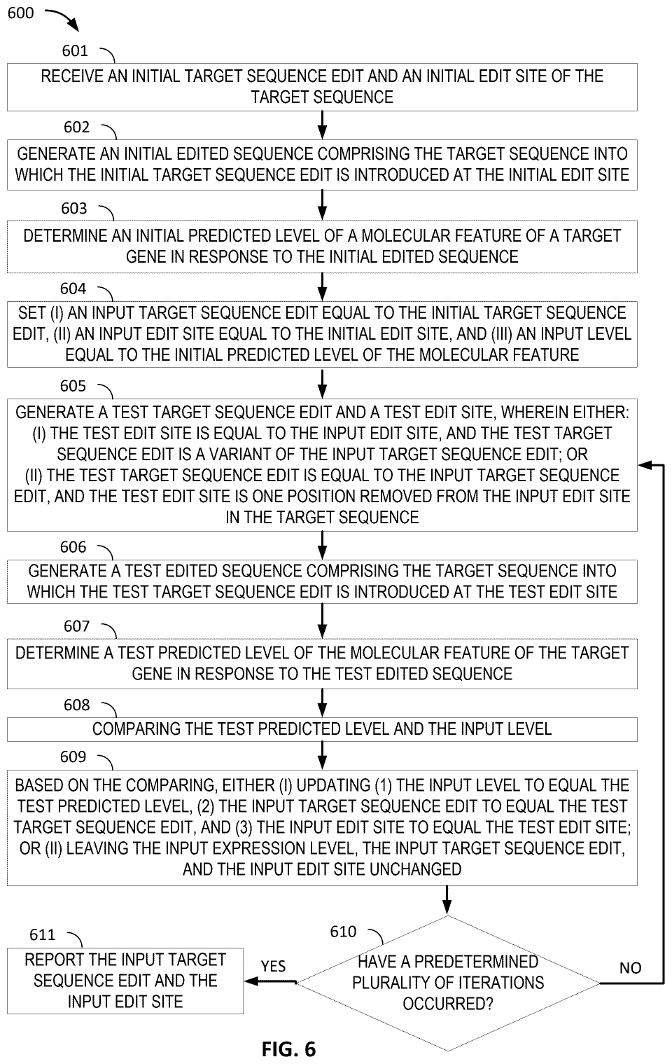

FIG. 6 presents a flowchart of a provided method 600 for designing a target sequence edit for introducing to a target sequence of a nucleic acid molecule. The target sequence edit can be any of those described in relation to method 400. In some embodiments, the target sequence edit is an insertion sequence. The target sequence can be any of those described in relation to block 401. In some embodiments, the target sequence includes a cis-regulatory element (CRE) operably linked to a target gene. An example of method 600 is described in Example 5. Method 600 can be performed partially or entirely using a computer system.

At block 601 of method 600, an initial target sequence edit and an initial edit site of the target sequence are received. In some embodiments, receiving the initial target sequence edit includes randomly generating the initial target sequence edit. Alternatively the initial target sequence edit can be received by, for example, choosing transcription factor motifs that are known and/or predicted to increase or decrease gene expression, e.g. using existing tools. The initial target sequence edit can be received by using predictive models (e.g. ENFORMER, BPNet, DeepSTARR, SEI) to predict specific synthetic sequences that will have desired effects on gene expression (Avsec et al., Nat. Methods 18, (2021): 1196). The initial target sequence edit can be received by pre-screening for sequence edits that would be likely to up-regulate or down-regulate gene expression by specific amounts using plasmid-based reporter assays or other reporter assays. The initial target sequence edit can be received by changing specific noncoding variants that occur in a given person back to the sequence present in the reference genome sequence, in order to restore expression to its normal level. The initial target sequence edit can be received by designing insertions (1-100+ nucleotides in length) of specific or random sequences, where the maximum length is specified as sequences that can be PCR-amplified from the genome. The initial target sequence edit can be received by designing sequence deletions (1-100+ nucleotides in length). The initial target sequence edit can be received by designing sequence inversions that might change the direction of a regulatory sequence or CTCF site (2-100+ nucleotides in length), where the maximum length is specified as sequences that can be PCR-amplified from the genome. The initial target sequence edit can be received by designing edits to existing sequence motifs to change binding affinity for regulatory factors including transcriptional activators and repressors. These edits could potentially include (1) changing nucleotides within an existing transcription factor binding site motif and (2) modifying the sequences that flank a transcription factor binding site motif.

At block 602 of method 600, an initial edited sequence is generated, where the initial edited sequence includes the target sequence into which the initial target sequence edit is introduced at the initial edit site.

At block 603 of method 600, an initial predicted level of a molecular feature of a target gene in response to the initial edited sequence is determined. In some embodiments, the molecular feature includes a gene expression level. In some embodiments, the molecular feature includes a binding of a transcription factor. In some embodiments, the molecular feature includes an epigenetic marker. In some embodiments, the molecular feature includes an accessibility to a chromatin. The initial predicted level of the molecular feature can be determined using sequence-based genomics assays that detect molecular features including transcription factor binding and epigenetic marks (chromatin immunoprecipitation), gene expression (cap analysis of gene expression, RNA-sequencing), and chromatin accessibility (assay for transposase-accessible chromatin with sequencing, DNase I hypersensitive sites sequencing). In some embodiments, the initial predicted level of the molecular feature includes use of Cap Analysis of Gene Expression (CAGE).

At block 604 of method 600, (i) an input target sequence edit is set equal to the initial target sequence edit, (ii) an input edit site is set equal to the initial edit site, and (iii) an input level is set equal to the initial predicted level of the molecular feature.

At block 605 of method 600, a test target sequence edit and a test edit site are generated, where either (i) the test edit site is equal to the input edit site, and the test target sequence edit is a variant of the input target sequence edit; or (ii) the test target sequence edit is equal to the input target sequence edit, and the test edit site is one position removed from the input edit site in the target sequence.

In some embodiments, the operation of block 605 includes generating a test target sequence edit and a test edit site, where either: (i) the test edit site is equal to the input edit site; and the test target sequence edit is equal to (1) the input target sequence edit from which one nucleotide is deleted, (2) the input target sequence edit in which one nucleotide is substituted, or (3) the input target sequence edit into which one nucleotide is inserted; or (ii) the test target sequence edit is equal to the input target sequence edit, and the test edit site is one position removed from the input edit site in the target sequence.

In some embodiments, the initial target sequence edit has a length no greater than a threshold length, and the operation of block 605 includes generating a test target sequence edit and a test edit site, where either: (i) the test edit site is equal to the input edit site; and the test target sequence edit is equal to (1) the input target sequence edit from which one nucleotide is deleted, (2) the input target sequence edit in which one nucleotide is substituted, or, if the input target sequence edit has a length at least one bp shorter than the threshold length, (3) the input target sequence edit into which one nucleotide is inserted; or (ii) the test target sequence edit is equal to the input target sequence edit, and the test edit site is one position removed from the input target sequence edit in the target sequence.

In some embodiments, the threshold length of the target sequence edit is between 3 bp and 35 bp. The threshold length can be, for example, between 3 bp and 13 bp, between 4 bp and 17 bp, between 5 bp and 21 bp, between 6 bp and 27 bp, or between 8 bp and 35 bp. In terms of upper limits, the threshold length can be, for example, no more than 35 bp, e.g., no more than 27 bp, no more than 21 bp, no more than 17 bp, no more than 13 bp, no more than 10 bp, no more than 8 bp, no more than 6 bp, no more than 5 bp, no more than 4 bp, or no more than 3 bp. In terms of lower limits, the threshold length can be, for example, at least 3 bp, e.g., at least 4 bp, at least 5 bp, at least 6 bp, at least 8 bp, at least 10 bp, at least 13 bp, at least 17 bp, at least 21 bp, at least 27 bp, or at least 35 bp.

At block 606 of method 600, a test edited sequence is generated, where the test edited sequence includes the target sequence into which the test target sequence edit is introduced at the test edit site.

At block 607 of method 600, a test predicted level of the molecular feature of the target gene in response to the test edited sequence is determined. The test predicted level of the molecular feature can be determined using sequence-based genomics assays that detect molecular features including transcription factor binding and epigenetic marks (chromatin immunoprecipitation), gene expression (cap analysis of gene expression, RNA-sequencing), and chromatin accessibility (assay for transposase-accessible chromatin with sequencing, DNase I hypersensitive sites sequencing). In some embodiments, the test predicted level of the molecular feature includes use of Cap Analysis of Gene Expression (CAGE).

At block 608 of method 600, the test predicted level and the input level are compared.

At block 609 of method 600, based on the comparing, either (i) (1) the input level is updated to equal the test predicted level, (2) the input target sequence edit is updated to equal the test target sequence edit, and (3) the input edit site is updated to equal the test edit site; or (ii) the input expression level, the input target sequence edit, and the input edit site are each left unchanged.

At block 610 of method 600, a decision is made based on whether or not a predetermined plurality of iterations has occurred. If the plurality of iterations has occurred, then the method proceeds to block 611. Otherwise, the method returns to block 605. In some embodiments, the plurality of iterations consists of between 10 and 1000 iterations. The number of iterations can be, for example, between 10 and 160, between 15 and 250, between 25 and 400, between 40 and 630, or between 63 and 1000. In terms of upper limits, the number of iterations ca be, for example, no more than 1000, e.g., no more than 630, no more than 400, no more than 250, no more than 160, no more than 100, no more than 63, no more than 40, no more than 25, no more than 15, or no more than 10. In terms of lower limits, the number of iterations can be, for example, at least 10, e.g., at least 15, at least 25, at least 40, at least 63, at least 100, at least 160, at least 250, at least 400, at least 630, or at least 1000.

At block 611 of method 600, the input target sequence edit and the input edit site following the iterations are reported, i.e., as output values of the target sequence edit and the edit site.

In some embodiments, method 600 further includes a step of synthesizing a nucleic acid molecule including the reported input target sequence edit. The nucleic acid molecule can be, for example, a pegRNA molecule. The peg RNA molecule can have any of the characteristics described in Section III.

### V. Compositions

In another aspect, the present disclosure provides compositions including a plurality of any of the template polynucleotides disclosed herein. For example, a provided composition can include a plurality of pegRNA molecules as described herein. The composition can be a pharmaceutical composition configured to better enable the delivery of the template polynucleotides, e.g., pegRNA molecules, to a subject.

In some embodiments, the composition is a pharmaceutical composition including a therapeutically effective amount of a pharmaceutically acceptable excipient. In some embodiments, the pharmaceutical composition includes one or more of a diluent, adjuvant, or carrier in a formulation suitable for administration, e.g., administration to a mammal. Suitable diluents, adjuvants, or carriers can include, for example, lipids, e.g., liposomes, e.g., liposome dendrimers; liquids, such as water and oils, including those of petroleum, animal, vegetable, or synthetic origin, such as peanut oil, soybean oil, mineral oil, sesame oil and the like; gum acacia; gelatin; starch paste; talc; keratin; colloidal silica; urea; and the like. Additional examples of suitable diluents include distilled water, buffered water, physiological saline, PBS, Ringer's solution, dextrose solution, and Hank's solution. The pharmaceutical compositions can also include additional substances to approximate physiological conditions, such as pH adjusting and buffering agents, toxicity adjusting agents, wetting agents, and detergents. In addition, auxiliary, thickening, lubricating, and coloring agents can alternatively or additionally be used. Pharmaceutical compositions can be formulated into preparations in solid, semisolid, liquid, or gaseous forms, such as tablets, capsules, powders, granules, ointments, solutions, suppositories, injections, inhalants, gels, microspheres, and aerosols.

A provided pharmaceutical composition can additionally or alternatively include any of a variety of stabilizing agents, such as an antioxidant for example. When the pharmaceutical composition includes a polynucleotide, the polynucleotide can be complexed with various well-known compounds or structures that enhance the in vivo stability or activity of the polynucleotide, or otherwise enhance its pharmacological properties (e.g., increase the half-life of the polynucleotide, reduce its toxicity, and/or enhance solubility or uptake). Examples of such modifications or complexing agents include lipids (e.g., ionic lipids), nanoparticles, lipid nanoparticles, exosomes, plasmids, vectors, and viruses (e.g., lentiviruses).

### VI. Methods for Treating or Preventing a Disease

In another aspect, the present disclosure provides methods for preventing or treating a disease. The methods generally include administering to a subject any of the any of the template polynucleotides disclosed herein, e.g., in Sections III and IV. Pharmaceutical compositions or cells containing the template polynucleotides, e.g., pegRNA molecules, can be administered for prophylactic and/or therapeutic treatments. In therapeutic applications, these compositions can be administered to a subject already suffering from a disease or condition, in an amount sufficient to cure or at least partially arrest the symptoms of the disease or condition, or to cure, heal, improve, or ameliorate the condition. Amounts effective for this use can vary based on the severity and course of the disease or condition, previous therapy, the subject's health status, weight, and response to the drugs, and the judgment of the treating physician.

The template polynucleotides described herein can be administered before, during, or after the occurrence of a disease or condition, and the timing of administering the composition can vary. For example, a pharmaceutical compositions can be used as a prophylactic and can be administered continuously to subjects with a propensity to conditions or diseases in order to prevent the occurrence of the disease or condition. The pharmaceutical compositions can be administered to a subject during or as soon as possible after the onset of the symptoms. The administration can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A composition can be administered as soon as is practicable after the onset of a disease or condition is detected or suspected, and for a length of time necessary for the treatment of the disease, such as, for example, from about 1 month to about 3 months. The length of treatment can vary for each subject.

A wide variety of diseases can be prevented or treated using the provided methods. The methods are particularly suitable for broad cell or gene therapy applications to treat a disease dependent on expression of the target gene that is edited by the administered template polynucleotides. In some embodiments, the method downregulates PPIF for treatment of inflammatory bowel disease. Inflammatory bowel disease (IBD) is a group of inflammatory conditions of the colon and small intestine, principally including Crohn's disease and ulcerative colitis, with other forms of IBD representing far fewer cases (e.g., collagenous colitis, lymphocytic colitis, diversion colitis, Behcet's disease and indeterminate colitis). Pathologically, Crohn's disease affects the full thickness of the bowel wall (e.g., transmural lesions) and can affect any part of the gastrointestinal tract, while ulcerative colitis is restricted to the mucosa (epithelial lining) of the colon and rectum. In certain embodiments, the IBD is Crohn's disease or ulcerative colitis. In certain embodiments, the IBD is collagenous colitis, lymphocytic colitis, diversion colitis, Behcet's disease, or indeterminate colitis.

In some embodiments, the provided treatment method is effective in upregulating the expression of genes in which mutations are known to cause haploinsufficiencies (through a mechanism not involving dominant negative effects of the disease-causing), for example including up-regulating ELN in endothelial cells, smooth muscle cells, and other cell types for Williams Syndrome; up-regulating JAG1 or NOTCH2 for Alagille Syndrome; and other haploinsufficiencies. In some embodiments, the provided treatment method is effective in modulating the expression of genes known to act as modifiers of rare or Mendelian diseases, for example including down-regulating BCL11A expression in red blood cell progenitors for treatment of sickle cell disease or thalassemias In some embodiments, the provided treatment method is effective in upregulating globin genes (including adult or fetal hemoglobins) or down-regulating BCL11A in red blood cell progenitors for sickle cell or beta thalassemia In some embodiments, the provided treatment method is effective in upregulating LDLR in hepatocytes for hypercholesterolemia and coronary artery disease. In some embodiments, the provided treatment method is effective in down-regulating CCM2 or TLNRD1 in arterial endothelial cells for coronary artery disease. In some embodiments, the provided treatment method is effective in upregulating NOS3 in arterial endothelial cells for coronary artery disease or hypertension. In some embodiments, the provided treatment method is effective in upregulating PLPP3 in arterial endothelial cells for coronary artery disease. In some embodiments, the provided treatment method is effective in downregulating PLPPR4 in endocardial cells for aortic valve calcification or stenosis. In some embodiments, the provided treatment method is effective in upregulating GOSR2 in endothelial cells for coronary artery disease. In some embodiments, the provided treatment method is effective in downregulating IL2RA in T Lymphocytes for inflammatory bowel disease. In some embodiments, the provided treatment method is effective in modulating the expression of immune checkpoint receptors on engineered immune cells (e.g. CAR-T cells), for example using regulatory sequences that are activated specifically in the tumor microenvironment but not in other normal tissues. In some embodiments, the provided treatment method is effective in modulating the expression of any gene of interest linked to a phenotypic trait in plants or animals.

In some embodiments, the provided method further includes obtaining a test sample from the subject. The test sample can include, for example, a blood sample, a tissue sample, a urine sample, a saliva sample, a cerebrospinal fluid sample, or a combination thereof. In some embodiments, the provided method further includes determining the level of one or more biomarkers in the obtained test sample. Determining the presence or level of biomarkers(s) can be used to, as non-limiting examples, determine response to treatment or to select an appropriate composition for the prevention or treatment of the disease.

In some embodiments, the provided method further includes comparing the determined level of the one of more biomarkers in the obtained test sample to the level of the one or more biomarkers in a reference sample. The reference sample can be obtained, for example, from the subject, with the reference sample being obtained prior to the obtaining of the test sample, e.g., prior to the administering to the subject of the therapeutically effective amount of the provided materials. In this way, the reference sample can provide information about baseline levels of the biomarkers in the sample before the treatment, and the test sample can provide information about levels of the biomarkers after the treatment.

Alternatively, the reference sample can be obtained, for example, from a different subject, e.g., a subject in which the treatment is not provided according to the provided methods. In this way, the reference sample can provide information about baseline levels of the biomarkers without treatment, and the test sample can provide information about levels of the biomarkers with treatment. The reference sample can also be obtained, for example, from a population of subjects, e.g., subjects in which the treatment is not provided according to the provided method. In this way, the reference sample can provide population-averaged information about baseline levels of the biomarkers without treatment, and the test sample can provide information about levels of the biomarkers with treatment.

The reference sample can also be obtained from an individual or a population of individuals after treatment is provided according to the provided methods, and can serve as, for example, a positive control sample. In some embodiments, the reference sample is obtained from normal tissue. In some embodiments, the reference sample is obtained from abnormal tissue.

Depending on the biomarker, an increase or a decrease relative to a normal control or reference sample can be indicative of the presence of a disease, or response to treatment for a disease. In some embodiments, an increased level of a biomarker in a test sample, and hence the presence of a disease, e.g., an infectious disease or cancer, increased risk of the disease, or response to treatment is determined when the biomarker levels are at least, 1.1-fold, e.g., at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, or at least 20-fold higher in comparison to a negative control. In other embodiments, a decreased level of a biomarker in the test sample, and hence the presence of the disease, increased risk of the disease, or response to treatment is determined when the biomarker levels are at least 1.1-fold, e.g., at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, or at least 20-fold lower in comparison to a negative control.

The biomarker levels can be detected using any method known in the art, including the use of antibodies specific for the biomarkers. Exemplary methods include, without limitation, polymerase chain reaction (PCR), Western Blot, dot blot, ELISA, radioimmunoassay (RIA), immunoprecipitation, immunofluorescence, FACS analysis, electrochemiluminescence, and multiplex bead assays, e.g., using Luminex or fluorescent microbeads. In some instances, nucleic acid sequencing is employed.

In certain embodiments, the presence of decreased or increased levels of one or more biomarkers is indicated by a detectable signal, e.g., a blot, fluorescence, chemiluminescence, color, or radioactivity, in an immunoassay or PCR reaction, e.g., quantitative PCR. This detectable signal can be compared to the signal from a reference sample or to a threshold value.

In some embodiments, the results of the biomarker level determinations are recorded in a tangible medium. For example, the results of diagnostic assays, e.g., the observation of the presence or decreased or increased presence of one or more biomarkers, and the diagnosis of whether or not there is an increased risk or the presence of a disease, e.g., an infectious disease or cancer, or whether or not a subject is responding to treatment can be recorded, for example, on paper or on electronic media, e.g., audio tape, a computer disk, a CD-ROM, or a flash drive.

In some embodiments, the provided method further includes the step of providing to the subject a diagnosis and/or the results of treatment.

### VII. Systems

In another aspect, the present disclosure provides various systems, e.g., measurement systems and/or computer systems, for performing the methods described herein, or individual or combined operations of those methods.

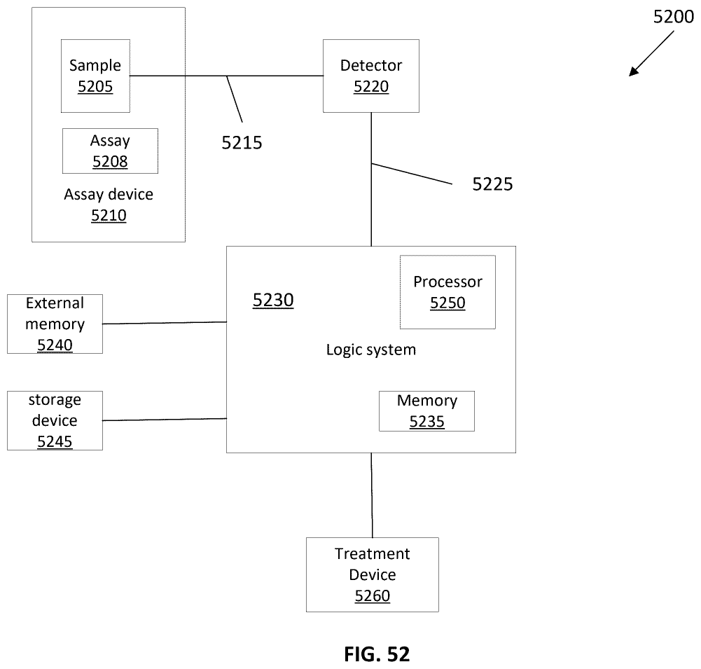

FIG. 52 illustrates a measurement system 5200 according to an embodiment of the present disclosure. The system as shown includes a sample 5205, such as sorted cells within an assay device 5210, where an assay 5208 can be performed on sample 5205. For example, sample 5205 can be contacted with reagents of assay 5208 to provide a signal of a physical characteristic 5215. Physical characteristic 5215 from the sample is detected by detector 5220. Detector 5220 can take a measurement at intervals (e.g., periodic intervals) to obtain data points that make up a data signal. In one embodiment, an analog-to-digital converter converts an analog signal from the detector into digital form at a plurality of times. Assay device 5210 and detector 5220 can form an assay system. A data signal 5225 is sent from detector 5220 to logic system 5230. As an example, data signal 5225 can be used to determine sequence information or gene expression. Data signal 5225 can include various measurements made at a same time, e.g., different colors of fluorescent dyes or different electrical signals for a different molecule or cell of sample 5205, and thus data signal 5225 can correspond to multiple signals. Data signal 5225 may be stored in a local memory 5235, an external memory 5240, or a storage device 5245.

Logic system 5230 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 5230 and the other components may be part of a stand-alone or network connected computer system, or they may be directly attached to or incorporated in a device (e.g., a sequencing device) that includes detector 5220 and/or assay device 5210. Logic system 5230 may also include software that executes in a processor 5250. Logic system 5230 may include a computer readable medium storing instructions for controlling measurement system 5200 to perform any of the methods described herein. For example, logic system 5230 can provide commands to a system that includes assay device 5210 such that sequencing operations are performed. Such physical operations can be performed in a particular order, e.g., with reagents being added and removed in a particular order. Such physical operations may be performed by a robotics system, e.g., including a robotic arm, as may be used to obtain a sample and perform an assay.

Measurement system 5200 may also include a treatment device 5260, which can provide a treatment to the subject. Treatment device 5260 can determine a treatment and/or be used to perform a treatment. Logic system 5230 may be connected to treatment device 5260, e.g., to provide results of a method described herein. The treatment device may receive inputs from other devices, such as an imaging device and user inputs (e.g., to control the treatment, such as controls over a robotic system).

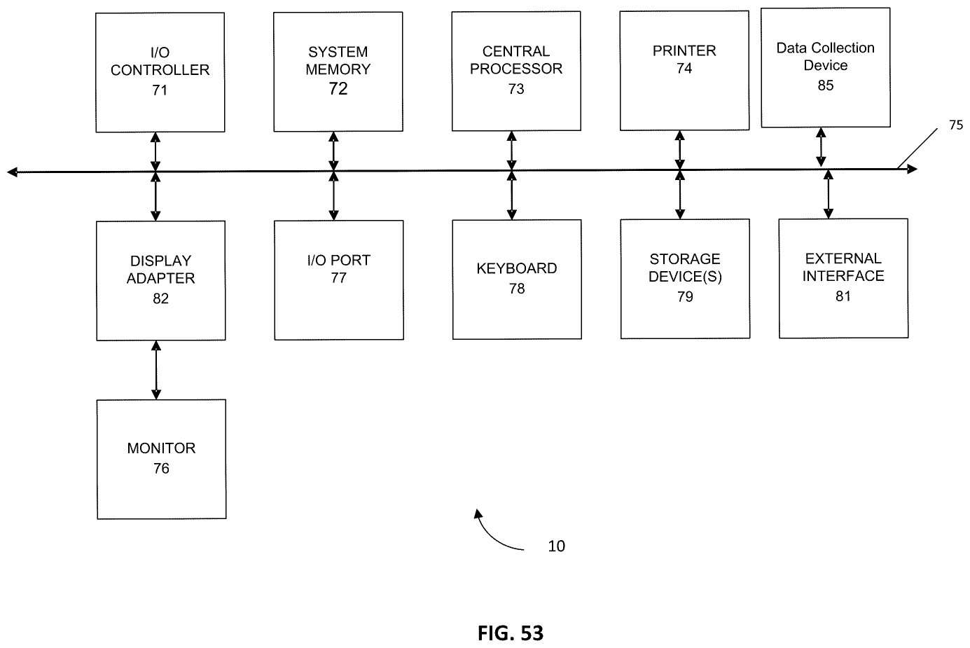

Any of the computer systems mentioned herein may utilize any suitable number of subsystems. Examples of such subsystems are shown in FIG. 53 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 53 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FIREWIRE©). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components. In various embodiments, methods may involve various numbers of clients and/or servers, including at least 10, 20, 50, 100, 200, 500, 1,000, or 10,000 devices. Methods can include various numbers of communications between devices, including at least 100, 200, 500, 1,000, 10,000, 50,000, 100,000, 500,000, or one million communications. Such communications can involve at least 1 MB, 10 MB, 100 MB, 1 GB, 10 GB, or 100 GB of data.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor may be performed in real-time. The term “real-time” may refer to computing operations or processes that are completed within a certain time constraint. The time constraint may be 1 minute, 1 hour, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

### VIII. Exemplary Embodiments

The following embodiments are contemplated. All combinations of features and embodiments are contemplated.

Embodiment 1: A method of screening edited genomic sequences for their effect on expression of a target gene, the method comprising: providing a population of cells, the genomes of the population of cells comprising a target sequence; introducing a plurality of different target sequence edits into the genomes of the population of cells; measuring a parameter for each cell of the population of cells, the parameter correlating with an expression level of the target gene; separating the population of cells into a plurality of sets based on the measured parameter; for each combination of (a) a set of the plurality of sets and (b) a target sequence edit of the plurality of different target sequence edits, determining a count of the target sequence edit incorporated into the genomes of the cells of the population of cells in the set; and for each target sequence edit of the plurality of different target sequence edits, based on the counts in the plurality of sets, computing an effect of the target sequence edit on the expression level of the target gene.

Embodiment 2: An embodiment of embodiment 1, wherein: the population of cells contains a fusion protein, the fusion protein comprising a polymerase and a DNA targeting protein, the DNA targeting protein having a nickase activity and binding to the target sequence; the introducing of the plurality of different target sequence edits comprises introducing to the population of cells a plurality of template polynucleotides, the template polynucleotides each independently comprising a target sequence edit of the plurality of different target sequence edits; and in at least a portion of the population of cells, a template polynucleotide of the plurality of template polynucleotides is primed by a target sequence nicked by the DNA targeting protein, and copied into the genome by the polymerase.

Embodiment 3: An embodiment of embodiment 2, wherein a plurality of prime-editing guide RNA (pegRNA) molecules comprises the plurality of template polynucleotides.

Embodiment 4: An embodiment of embodiment 3, wherein the plurality of pegRNA molecules further comprises a gRNA spacer no more than 50 bp away from the target sequence edit.

Embodiment 5: An embodiment of embodiment 3 or 4, wherein the plurality of pegRNA molecules further comprises a scaffold sequence having at least 85% sequence identity with the sequence:

(SEQ ID NO: 1)
GTTTAAGAGCTATGCTGGAAACAGCATAGCAAGTTTAAATAAGGCTAGTC
CGTTATCAACTTGAAAAAGTGGCACCGAGTCGGTGC.

Embodiment 6: An embodiment of any one of embodiments 3-5, wherein the plurality of pegRNA molecules further comprises a primer-binding site (PBS) having a length between 6 bp and 16 bp.

Embodiment 7: An embodiment of any one of embodiments 3-6, wherein the plurality of pegRNA molecules further comprises a reverse transcription (RT) template having a length no greater than 60 bp.

Embodiment 8: An embodiment of embodiment 7, wherein the reverse transcription (RT) template extends between 6 bp and 16 bp upstream of the target sequence edit.

Embodiment 9: An embodiment of any one of embodiments 3-8, wherein the plurality of pegRNA molecules further comprises a 3′ end modification having at least 86% sequence identity with the 37-bp trimmed evopreRQ (tevopreQ1) pseudoknot sequence.

Embodiment 10: An embodiment of any one of embodiments 2-9, wherein the introducing of the plurality of template polynucleotides comprises use of a lentivirus, a transposase, or a recombinase.

Embodiment 11: An embodiment of embodiment 10, wherein the introducing of the plurality of template polynucleotides comprises transducing the population of cells with lentivirus.

Embodiment 12: An embodiment of any one of embodiments 2-11, wherein the method further comprises introducing to the population of cells an inducer of expression of the fusion protein.

Embodiment 13: An embodiment of embodiment 12, wherein the population of cells comprises cells of an inducible Cas9 RT-nickase cell line.

Embodiment 14: An embodiment of any one of embodiments 1-13, wherein the determining of the counts of the plurality of different target sequence edits comprises: receiving a plurality of sequence reads; for each of the plurality of sequence reads, determining if the sequence read belongs to an aligning set consisting of the sequence reads of the plurality of sequence reads that align to a reference amplicon, the reference amplicon comprising the target sequence; for each variant sequence of a set of variant sequences, determining a count of the sequence reads of the aligning set that comprise the variant sequence, each variant sequence of the set of variant sequences independently comprising the target sequence modified according to a target sequence edit of the plurality of different target sequence edits; and determining a count of the sequence reads of the aligning set that do not match any variant sequence of the set of variant sequences, and that differ from the target sequence by no more than a threshold percentage of base pairs.

Embodiment 15: An embodiment of embodiment 14, wherein the threshold percentage is no greater than 15%.

Embodiment 16: An embodiment of any one of embodiments 1-15, wherein the computing of the effect of the target sequence edit comprises: for each target sequence edit of the plurality of different target sequence edits, fitting the counts in the plurality of sets to a log-normal distribution; and calculating a percent difference between (a) the log-normal distribution for the target sequence edit, and (b) a log-normal distribution derived by fitting counts of the target sequence in the plurality of sets.

Embodiment 17: An embodiment of any one of embodiments 1-16, wherein the target sequence comprises a cis-regulatory element (CRE) operably linked to the target gene.

Embodiment 18: An embodiment of any one of embodiments 1-17, wherein the plurality of different target sequence edits each independently comprise one or more substitutions, insertions, deletions, or a combination thereof, relative to the target sequence.

Embodiment 19: An embodiment of any one of embodiments 1-18, wherein the determining of the count of the plurality of different target sequence edits comprises sequencing a target region of the genomes of the population of cells, the target region comprising the target sequence prior to the introducing of the plurality of different target sequence edits into the population of cells.

Embodiment 20: An embodiment of any one of embodiments 1-19, wherein the plurality of different target sequence edits comprises at least 3 different target sequence edits.

Embodiment 21: An embodiment of any one of embodiments 1-20, wherein the plurality of sets comprises at least 3 sets.

Embodiment 22: An embodiment of any one of embodiments 1-21, wherein the measuring of the parameter comprises using a phenotypic assay.

Embodiment 23: An embodiment of embodiment 22, wherein the phenotypic assay comprises a fluorescence-in-situ-hybridization (FISH) assay, a fluorescence assay, an immunofluorescence assay, a growth assay, or a combination thereof.

Embodiment 24: An embodiment of any one of embodiments 1-23, wherein the separating of the population of cells comprises fluorescence activated cell sorting (FACS).

Embodiment 25: A method of designing a target sequence edit for introducing to a target sequence of a nucleic acid molecule, the method comprising: receiving an initial target sequence edit and an initial edit site of the target sequence; generating an initial edited sequence comprising the target sequence into which the initial target sequence edit is introduced at the initial edit site; determining an initial predicted level of a molecular feature of a target gene in response to the initial edited sequence; setting (i) an input target sequence edit equal to the initial target sequence edit, (ii) an input edit site equal to the initial edit site, and (iii) an input level equal to the initial predicted level of the molecular feature; performing a series of operations in an iterative manner for a plurality of iterations, the series of operations comprising: (a) generating a test target sequence edit and a test edit site, wherein either: (i) the test edit site is equal to the input edit site, and the test target sequence edit is a variant of the input target sequence edit; or (ii) the test target sequence edit is equal to the input target sequence edit, and the test edit site is one position removed from the input edit site in the target sequence; (b) generating a test edited sequence comprising the target sequence into which the test target sequence edit is introduced at the test edit site; (c) determining a test predicted level of the molecular feature of the target gene in response to the test edited sequence; (d) comparing the test predicted level and the input level; (e) based on the comparing, either (i) updating (1) the input level to equal the test predicted level, (2) the input target sequence edit to equal the test target sequence edit, and (3) the input edit site to equal the test edit site; or (ii) leaving the input expression level, the input target sequence edit, and the input edit site unchanged; and subsequent to the plurality of iterations, reporting the input target sequence edit and the input edit site.

Embodiment 26: An embodiment of embodiment 25, wherein operation (a) of the series of operations comprises (a) generating a test target sequence edit and a test edit site, wherein either: (i) the test edit site is equal to the input edit site; and the test target sequence edit is equal to (1) the input target sequence edit from which one nucleotide is deleted, (2) the input target sequence edit in which one nucleotide is substituted, or (3) the input target sequence edit into which one nucleotide is inserted; or (ii) the test target sequence edit is equal to the input target sequence edit, and the test edit site is one position removed from the input edit site in the target sequence.

Embodiment 27: An embodiment of embodiment 26, wherein: the initial target sequence edit has a length no greater than a threshold length; and operation (a) of the series of operations comprises (a) generating a test target sequence edit and a test edit site, wherein either: (i) the test edit site is equal to the input edit site; and the test target sequence edit is equal to (1) the input target sequence edit from which one nucleotide is deleted, (2) the input target sequence edit in which one nucleotide is substituted, or, if the input target sequence edit has a length at least one bp shorter than the threshold length, (3) the input target sequence edit into which one nucleotide is inserted; or (ii) the test target sequence edit is equal to the input target sequence edit, and the test edit site is one position removed from the input target sequence edit in the target sequence.

Embodiment 28: An embodiment of embodiment 27, wherein the threshold length is no greater than 35 bp.

Embodiment 29: An embodiment of any one of embodiments 26-28, wherein the target sequence edit is an insertion sequence.

Embodiment 30: An embodiment of any one of embodiments 25-29, wherein the molecular feature of the target gene comprises a gene expression level, a binding of a transcription factor, an epigenetic mark, or an accessibility to a chromatin.

Embodiment 31: An embodiment of embodiment 30, wherein the molecular feature of the target gene is an expression level of the target gene operably linked to the target sequence.

Embodiment 32: An embodiment of embodiment 31, wherein the target sequence is a cis-regulatory element (CRE) operably linked to the target gene.

Embodiment 33: An embodiment of embodiment 31 or 32, wherein the determining of the initial predicted level and the test predicted level comprises use of Cap Analysis of Gene Expression (CAGE).

Embodiment 34: An embodiment of any one of embodiments 25-33, wherein the receiving of the initial target sequence edit comprising randomly generating the initial target sequence edit.

Embodiment 35: An embodiment of any one of embodiments 25-34, wherein the plurality of iterations consists of at least 10 iterations.

Embodiment 36: An embodiment of any one of embodiments 25-35, wherein the method further comprises synthesizing a nucleic acid molecule comprising the reported input target sequence edit.

Embodiment 37: An embodiment of embodiment 36, wherein the nucleic acid molecule is a pegRNA molecule.

Embodiment 38: An embodiment of embodiment 37, wherein the pegRNA molecule comprises a gRNA spacer no more than 50 bp away from the reported target sequence edit.

Embodiment 39: An embodiment of embodiment 37 or 38, wherein the pegRNA molecule comprises a scaffold sequence having at least 85% sequence identity with the sequence:

(SEQ ID NO: 1)
GTTTAAGAGCTATGCTGGAAACAGCATAGCAAGTTTAAATAAGGCTAGTC
CGTTATCAACTTGAAAAAGTGGCACCGAGTCGGTGC.

Embodiment 40: An embodiment of any one of embodiments 37-39, wherein the pegRNA molecule comprises a primer-binding site (PBS) having a length between 6 bp and 16 bp.

Embodiment 41: An embodiment of any one of embodiments 37-40, wherein the pegRNA molecule comprises a reverse transcription (RT) template having a length no greater than 60 bp.

Embodiment 42: An embodiment of any one of embodiments 37-41, wherein the reverse transcription (RT) template extends between 6 bp and 16 bp upstream of the reported input insertion sequence.

Embodiment 43: An embodiment of any one of embodiments 37-42, wherein the pegRNA molecule comprises a 3′ end modification having at least 86% sequence identity with the 37-bp trimmed evopreRQ (tevopreQ1) pseudoknot sequence.

Embodiment 44: An embodiment of embodiment 3, wherein the plurality of pegRNA molecules comprises the pegRNA molecule of any one of claims 37-43.

Embodiment 45: A composition comprising the plurality of template polynucleotides of any one of claims 2-9.

Embodiment 46: A population of cells comprising the plurality of template polynucleotides of any one of claims 2-9.

Embodiment 47: A cell comprising a genome incorporating a target sequence edit of the plurality of different target sequence edits of any one of embodiments 1-24, or incorporating the reported input target sequence edit of any one of embodiments 25-44.

Embodiment 48: An embodiment of embodiment 47, wherein the cell is a monocyte or T cell.

Embodiment 49: A method of treating a subject having a disease dependent on expression of the target gene of any one of embodiments 1-44, the method comprising administering to the subject a therapeutically effective amount of the plurality of template polynucleotides of any one of embodiment 2-9, or a therapeutically effective amount of a population of cells comprising the cell of embodiment 47 or 48.

Embodiment 50: An embodiment of embodiment 49, wherein the target gene encodes peptidylprolyl isomerase F (PPIF).

Embodiment 51: An embodiment of embodiment 50, wherein the disease comprises inflammatory bowel disease.

Embodiment 52: A computer product comprising a non-transitory computer readable medium storing a plurality of instructions that, when executed, cause a computer system to perform the method of any one of embodiments 1-44.

Embodiment 53: A system comprising: the computer product of embodiment 52; and one or more processors for executing instructions stored on the computer readable medium.

Embodiment 54: A method of screening edited genomic sequences for their effect on expression of a target gene, the method comprising: providing a population of cells, the genomes of the population of cells comprising a target sequence; introducing a plurality of different target sequence edits into the genomes of the population of cells; measuring a parameter for each cell of the population of cells, the parameter correlating with an expression level of the target gene; separating the population of cells into a plurality of sets based on the measured parameter; for each combination of (a) a set of the plurality of sets and (b) a target sequence edit of the plurality of different target sequence edits, determining a count of the target sequence edit incorporated into the genomes of the cells of the population of cells in the set; and for each target sequence edit of the plurality of different target sequence edits, based on the counts in the plurality of sets, computing an effect of the target sequence edit on the expression level of the target gene.

Embodiment 55: An embodiment of embodiment 54, wherein the plurality of different target sequence edits comprises a designed target sequence edit identified by a simulation process, the simulation process comprising: receiving an initial target sequence edit and an initial edit site of the target sequence; generating an initial edited sequence comprising the target sequence into which the initial target sequence edit is introduced at the initial edit site; determining an initial predicted level of a molecular feature of a target gene in response to the initial edited sequence; setting (i) an input target sequence edit equal to the initial target sequence edit, (ii) an input edit site equal to the initial edit site, and (iii) an input level equal to the initial predicted level of the molecular feature; performing a series of operations in an iterative manner for a plurality of iterations, the series of operations comprising: (a) generating a test target sequence edit and a test edit site, wherein either: (i) the test edit site is equal to the input edit site, and the test target sequence edit is a variant of the input target sequence edit; or (ii) the test target sequence edit is equal to the input target sequence edit, and the test edit site is one position removed from the input edit site in the target sequence; (b) generating a test edited sequence comprising the target sequence into which the test target sequence edit is introduced at the test edit site; (c) determining a test predicted level of the molecular feature of the target gene in response to the test edited sequence; (d) comparing the test predicted level and the input level; (e) based on the comparing, either (i) updating (1) the input level to equal the test predicted level, (2) the input target sequence edit to equal the test target sequence edit, and (3) the input edit site to equal the test edit site; or (ii) leaving the input expression level, the input target sequence edit, and the input edit site unchanged; and subsequent to the plurality of iterations, reporting the input target sequence edit as the designed target sequence edit.

Embodiment 56: An embodiment of embodiment 55, wherein operation (a) of the series of operations comprises (a) generating a test target sequence edit and a test edit site, wherein either: (i) the test edit site is equal to the input edit site; and the test target sequence edit is equal to (1) the input target sequence edit from which one nucleotide is deleted, (2) the input target sequence edit in which one nucleotide is substituted, or (3) the input target sequence edit into which one nucleotide is inserted; or (ii) the test target sequence edit is equal to the input target sequence edit, and the test edit site is one position removed from the input edit site in the target sequence.

Embodiment 57: An embodiment of embodiment 56, wherein: the initial target sequence edit has a length no greater than a threshold length; and operation (a) of the series of operations comprises (a) generating a test target sequence edit and a test edit site, wherein either: (i) the test edit site is equal to the input edit site; and the test target sequence edit is equal to (1) the input target sequence edit from which one nucleotide is deleted, (2) the input target sequence edit in which one nucleotide is substituted, or, if the input target sequence edit has a length at least one bp shorter than the threshold length, (3) the input target sequence edit into which one nucleotide is inserted; or (ii) the test target sequence edit is equal to the input target sequence edit, and the test edit site is one position removed from the input target sequence edit in the target sequence.

Embodiment 58: An embodiment of embodiment 57, wherein the threshold length is no greater than 35 bp.

Embodiment 59: An embodiment of any one of embodiments 55-58, wherein the designed target sequence edit is an insertion sequence.

Embodiment 60: An embodiment of any one of embodiments 54-59, wherein the molecular feature of the target gene comprises a gene expression level, a binding of a transcription factor, an epigenetic mark, or an accessibility to a chromatin.

Embodiment 61: An embodiment of embodiment 60, wherein the molecular feature of the target gene is an expression level of the target gene operably linked to the target sequence.

Embodiment 62: An embodiment of embodiment 60 or 61, wherein the determining of the initial predicted level and the test predicted level comprises use of Cap Analysis of Gene Expression (CAGE).

Embodiment 63: An embodiment of any one of embodiments 55-62, wherein the receiving of the initial target sequence edit comprising randomly generating the initial target sequence edit.

Embodiment 64: An embodiment of any one of embodiments 55-63, wherein the plurality of iterations consists of at least 10 iterations.

Embodiment 65: An embodiment of any one of embodiments 55-64, wherein the method further comprises performing the simulation process, thereby identifying the designed target sequence edit.

Embodiment 66: An embodiment of any one of embodiments 54-65, wherein: the population of cells contains a fusion protein, the fusion protein comprising a polymerase and a DNA targeting protein, the DNA targeting protein having a nickase activity and binding to the target sequence; the introducing of the plurality of different target sequence edits comprises introducing to the population of cells a plurality of template polynucleotides, the template polynucleotides each independently comprising a target sequence edit of the plurality of different target sequence edits; and in at least a portion of the population of cells, a template polynucleotide of the plurality of template polynucleotides is primed by a target sequence nicked by the DNA targeting protein, and copied into the genome by the polymerase.

Embodiment 67: An embodiment of embodiment 66, wherein a plurality of prime-editing guide RNA (pegRNA) molecules comprises the plurality of template polynucleotides.

Embodiment 68: An embodiment of embodiment 67, wherein the plurality of pegRNA molecules further comprises a gRNA spacer no more than 50 bp away from the target sequence edit.

Embodiment 69: An embodiment of embodiment 67 or 68, wherein the plurality of pegRNA molecules further comprises a scaffold sequence having at least 85% sequence identity with the sequence:

(SEQ ID NO: 1)
GTTTAAGAGCTATGCTGGAAACAGCATAGCAAGTTTAAATAAGGCTAGTC
CGTTATCAACTTGAAAAAGTGGCACCGAGTCGGTGC.

Embodiment 70: An embodiment of any one of embodiments 67-69, wherein the plurality of pegRNA molecules further comprises a primer-binding site (PBS) having a length between 6 bp and 16 bp.

Embodiment 71: An embodiment of any one of embodiments 67-70, wherein the plurality of pegRNA molecules further comprises a reverse transcription (RT) template having a length no greater than 60 bp.

Embodiment 72: An embodiment of embodiment 71, wherein the reverse transcription (RT) template extends between 6 bp and 16 bp upstream of the target sequence edit.

Embodiment 73: An embodiment of any one of embodiments 67-72, wherein the plurality of pegRNA molecules further comprises a 3′ end modification having at least 86% sequence identity with the 37-bp trimmed evopreRQ (tevopreQ1) pseudoknot sequence.

Embodiment 74: An embodiment of any one of embodiments 66-73, wherein the introducing of the plurality of template polynucleotides comprises use of a lentivirus, a transposase, or a recombinase.

Embodiment 75: An embodiment of embodiment 74, wherein the introducing of the plurality of template polynucleotides comprises transducing the population of cells with lentivirus.

Embodiment 76: An embodiment of any one of embodiments 66-75, wherein the method further comprises introducing to the population of cells an inducer of expression of the fusion protein.

Embodiment 77: An embodiment of embodiment 76, wherein the population of cells comprises cells of an inducible Cas9 RT-nickase cell line.

Embodiment 78: An embodiment of any one of embodiments 54-77, wherein the determining of the counts of the plurality of different target sequence edits comprises: receiving a plurality of sequence reads; for each of the plurality of sequence reads, determining if the sequence read belongs to an aligning set consisting of the sequence reads of the plurality of sequence reads that align to a reference amplicon, the reference amplicon comprising the target sequence; for each variant sequence of a set of variant sequences, determining a count of the sequence reads of the aligning set that comprise the variant sequence, each variant sequence of the set of variant sequences independently comprising the target sequence modified according to a target sequence edit of the plurality of different target sequence edits; and determining a count of the sequence reads of the aligning set that do not match any variant sequence of the set of variant sequences, and that differ from the target sequence by no more than a threshold percentage of base pairs.

Embodiment 79: An embodiment of embodiment 78, wherein the threshold percentage is no greater than 15%.

Embodiment 80: An embodiment of any one of embodiments 54-79, wherein the computing of the effect of the target sequence edit comprises: for each target sequence edit of the plurality of different target sequence edits, fitting the counts in the plurality of sets to a log-normal distribution; and calculating a percent difference between (a) the log-normal distribution for the target sequence edit, and (b) a log-normal distribution derived by fitting counts of the target sequence in the plurality of sets.

Embodiment 81: An embodiment of any one of embodiments 54-80, wherein the target sequence comprises a cis-regulatory element (CRE) operably linked to the target gene.

Embodiment 82: An embodiment of any one of embodiments 54-81, wherein the plurality of different target sequence edits each independently comprise one or more substitutions, insertions, deletions, or a combination thereof, relative to the target sequence.

Embodiment 83: An embodiment of any one of embodiments 54-82, wherein the determining of the count of the plurality of different target sequence edits comprises sequencing a target region of the genomes of the population of cells, the target region comprising the target sequence prior to the introducing of the plurality of different target sequence edits into the population of cells.

Embodiment 84: An embodiment of any one of embodiments 54-83, wherein the plurality of different target sequence edits comprises at least 3 different target sequence edits.

Embodiment 85: An embodiment of any one of embodiments 54-84, wherein the plurality of sets comprises at least 3 sets.

Embodiment 86: An embodiment of any one of embodiments 54-85, wherein the measuring of the parameter comprises using a phenotypic assay.

Embodiment 87: An embodiment of embodiment 86, wherein the phenotypic assay comprises a fluorescence-in-situ-hybridization (FISH) assay, a fluorescence assay, an immunofluorescence assay, a growth assay, or a combination thereof.

Embodiment 88: An embodiment of any one of embodiments 54-87, wherein the separating of the population of cells comprises fluorescence activated cell sorting (FACS).

Embodiment 89: A composition comprising the plurality of template polynucleotides of any one of embodiments 66-73.

Embodiment 90: A population of cells comprising the plurality of template polynucleotides of any one of embodiments 66-73.

Embodiment 91: A cell comprising a genome incorporating a target sequence edit of the plurality of different target sequence edits of any one of embodiments 54-88.

Embodiment 92: An embodiment of embodiment 91, wherein the cell is a monocyte or T cell.

Embodiment 93: A method of treating a subject having a disease dependent on expression of the target gene of any one of embodiments 54-88, the method comprising administering to the subject a therapeutically effective amount of the plurality of template polynucleotides of any one of embodiments 66-73, or a therapeutically effective amount of a plurality of cells comprising the cell of embodiment 91 or 92.

Embodiment 94: An embodiment of embodiment 93, wherein the target gene encodes peptidylprolyl isomerase F (PPIF).

Embodiment 95: An embodiment of embodiment 94, wherein the disease comprises inflammatory bowel disease.

Embodiment 96: An embodiment of embodiment A computer product comprising a non-transitory computer readable medium storing a plurality of instructions that, when executed, cause a computer system to perform the method of any one of embodiments 1-88.

Embodiment 97: A system comprising: the computer product of claim 96; and one or more processors for executing instructions stored on the computer readable medium.

### EXAMPLES

The present disclosure will be better understood in view of the following non-limiting examples. The following examples are intended for illustrative purposes only and do not limit in any way the scope of the present invention.

### Example 1. Development of Experimental Protocols and Workflows

A. Cloning of pegRNA Pools
1. Amplification of pegRNAs from Oligo Pools

The pegRNA constructs were ordered as a pool of oligos from Agilent Technologies Inc. pegRNAs designed to target and scramble the sequence at each individual loci (e.g. enhancer, promoter, or negative control region) were ordered as an individual subpool. These subpools were PCR amplified first using primers specific to each of the subpool specific handles, before a second round of PCR was performed to add the arms necessary for Gibson Assembly into the sgOpti (puromycin resistance) vector. The oligo pool was diluted to 1 ng/μl for the first PCR. The 2× NEBNext Master Mix was used as the polymerase. A 1.2× Ampure XP SPRI clean was performed in between each round of PCR. Example primer sequences are shown in Table 1 below. For Variant FlowFISH experiments with principled design edit pools, the pegRNAs were cloned into a modified SgOpti vector containing the tevopreQ1 pseudoknot sequence (epegRNA) derived from Nelson et al., Nat. Biotechnol. 40, (2022): 402.

TABLE 1
Example primer sequences for amplification of pegRNA oligos

| PCR stage | Primer Name | Sequence |
|---|---|---|
| PCR1 | Subpool Fwd primer (wells A1) | GTCCTTGCCAATGAACGGGG (SEQ ID NO: 2) |
| | Subpool Rev primer (wells E1) | TTCAAGACGGGCGTGACTGC (SEQ ID NO: 3) |
| PCR2 | CI0111_CRISPRPoolAmp_F | ggctttatatatcttgtggaaaggacgaaacaccg (SEQ ID NO: 4) |
| | pSgOptipegAmp_R | gatgaatactgccatttgtctcaagatctag (SEQ ID NO: 5) |
| | or | |
| | epegRNA Rev tevoPQ1 | tggtttaacgcgtaactagatagaaccgcg (SEQ ID NO: 6) |

### 2. Digestion of Vector and Assembly

The sgOpti plasmid (puromycin resistance gene) was sequentially digested with the BsmBI (55° C. overnight digestion) and EcoRI/ClaI (37° C. overnight digestion) restriction enzymes. The fragments together were Gibson Assembled and a high complexity transformation of the plasmids into Lucigen Endura ElectroCompetent Cells was performed. Serial dilutions of the cells were performed and plated onto LB (carbenicillin resistance) plates: 1:1K, 1:10K, 1:100K, 1:1M, 1:10M, 1:100M. A negative control of the digested backbone was also included. A successful high complexity transformation was indicated by >1000 fold coverage of the library and >100 fold increase over the negative control (digested backbone control).

3. Verification of the pegRNA Pool and Skew Ratio

The pegRNA pools were PCR amplified and sequenced via an Illumina Platform (e.g. MiSeq) to determine the skew ratio for each of the pegRNA pools. Example primers required for this step and the custom index primers required to run these products on the MiSeq Illumina platform are shown in Table 2 below.

TABLE 2
Example primers to amplify and determine the skew ratio for pegRNA oligos
pools

| Step | Primer Name and Sequence |
|---|---|
| PCR | Fwd: Seq997 barcoded series |
| | AATGATACGGCGACCACCGAGATCTACACACGTCGATTTCTTGGCTTTATATATCTTGTG (SEQ |
| | ID NO: 7) |
| | Rev: sgPuroPrimeEditorSeqRev |
| | CAAGCAGAAGACGGCATACGAGATCCTGGTAG GGATGAATACTGCCATTTGTC (SEQ ID NO: 8) |
| | or |
| | Rev: sgPurotevoPQ_PrimeEditorSeqRev |
| | CAAGCAGAAGACGGCATACGAGATAGATGTGCATGTCATATGTAGAGAGGTACCT (SEQ ID NO: |
| | 9) |
| MiSeq | Custom Primer Into Port 18: Custom Read 1 Sequencing Primer: Seq999_hU6_R1 |
| | CGATTTCTTGGCTTTATATATCTTGTGGAAAGGACGAAACACCG (SEQ ID NO: 10) |
| | Into Port 19: Custom Index Primer: PE_sgSeq007_I1 |
| | gatcttgagacaaatggcagtattcatcc (SEQ ID NO: 11) |
| | Into Port [BLANK]: Custom Read 2 Sequencing Primer: PE_sgSeq007_R2 |
| | ggatgaatactgccatttgtctcaagatc (SEQ ID NO: 12) |

### B. Generating Lentivirus

Lentivirus containing each of the pegRNA pools was generated using HEK293T cells (cultured in DMEM, 10% hiFBS, without PS). Approximately 550,000 HEK293T cells were seeded the day before transfection in each well of a 6 well plate. On the day of transfection, 1200 ng of transfer plasmid (pegRNA pool) was combined with 900 ng of psPAX2 and 360 ng of pMD2.g (lentivirus packaging plasmids). A mixture of 192 μl of Opti-MEM was combined with 5.8 μl of XtremeGENE and let to incubate for 5 minutes, before combining with the DNA mixture (lentivirus packaging plasmids and transfer DNA). The DNA/XtremeGENE mixture was incubated for 15 minutes before dripping onto the cells in the 6-well dish. The cells were incubated for 48 hours at 37° C., before the virus/media of the cells was harvested and passed through a syringe with a 0.45 μM filter and stored for use when infecting the cells.

### C. Constructing the THP-1 Prime Editor Cell Line

A THP-1 cell line was constructed where the prime editing Cas9-reverse transcriptase nickase machinery (PE2 system) was incorporated into the genome under the control of a doxycycline inducible promoter using the Tet Response Element (TRE) construct TRE-PE2-IRES-BFP. This construct is also fused to a BFP expression cassette, such that BFP expression can be used to indicate the expression of the PE machinery. The cell line also has the reverse tetracycline-controlled transactivator (rtTA) neomycin resistance construct incorporated into the genome. rtTA interacts with doxycycline to induce expression of the PE2 system. THP-1 cells were treated with doxycycline (final concentration 1 μg/mL) for 48 hours to induce BFP expression and turn on the PE machinery. BFP enrichment sorts are regularly performed on this cell line to ensure that the THP-1 PE cell population retains a BFP expression level >90%. Other PE2 cell lines, including Jurkat PE2, K562 PE2, GM12878 PE2 and TeloHAEC PE2, have also been developed and used with the provided methods.

D. Titering and Spinfection with the Lentivirus

### 1. Titering the Lentivirus

The harvested lentivirus was titered to determine a MOI of 0.2 via spinfection. The amount of virus was titered to yield approximately one pegRNA per cell. Different volumes of virus were added to each well of a 12-well plate to determine the amount of virus required for an MOI of 0.2 (e.g. untreated control, 1 μl, 3 μl, 5 μl, 10 μl, etc.). Approximately 500,000 THP-1 PE2 cells (cultured in RPMI, 10% hiFBS, 1% PS, 2 mM L-Glutamine) were seeded per well of a 12-well plate with 8 μg/mL of polybrene per well. The appropriate volume of virus was added to each well and mixed. The plates were spun at 1200 g for 40 minutes at 37° C. to infect the cells. Approximately 24 hours after infection, puromycin selection (encoded on the pegRNA plasmid) was applied to the THP-1 PE2 cells at a final concentration of 1.5 μg/mL. Selection of the infected cells occurs over a 72 hour period before the viability of the cells is assessed via a hemocytometer. The titering determined the amount of virus (per 12-well) which allows 20% of the cells to survive, with less than 5% survival in the untreated control well. This amount was designated as having a MOI of 0.2.

2. Spinfection with the Lentivirus

THP-1 PE2 cells were infected at a MOI of 2 for the final infection of the cells for the Variant FlowFISH experiment (10× the volume of virus determined for a MOI of 0.2). This amount was selected because not every pegRNA is effective at introducing a variant into the genome, and if more than one pegRNA enters a cell, it is unlikely that more than one edit would be introduced at the target site. In terms of coverage, the aim was for at least ˜1000 cells with each variant, but to avoid bottle-necks during the culturing process between biological replicates, some extra coverage was desired and infection was carried out at 5000× coverage. Cells were spinfected following the protocol described in the virus titering section. Infections were performed with two biological replicates.

### 3. Selection of Infected Cells

Infected cells were selected with a final concentration of puromycin of 1.5 ug/mL for 72 hrs.

E. Treating Cells with Doxycycline

At 72 hrs, the cells were pooled together, spun down, and resuspended in fresh RPMI media (10% hiFBS, 1% PS, 2 mM L-Glutamine) containing a final concentration of 0.3 μg/mL puromycin and 1 μg/mL doxycycline. This maintenance dose of puromycin prevents infected cells from losing the pegRNAs over time. The doxycycline is added to induce the prime editing machinery (PE2 system with the Cas9-RT nickase). Editing was induced for a period of 10-14 days, and an increase in the total % of editing is observed with increasing time. Cells are maintained at the coverage they were initially infected at during the culturing process, with a minimum coverage of at least 50,000× (i.e., 50,000 cells per variant) during the culturing process.

### F. Extracting Genomic DNA Extraction and Checking for Editing Through Genotyping

After at least 10 days of doxycycline induction of the THP-1 or Jurkat PE2 cells, the genomic DNA (gDNA) is harvested from the cells. Briefly, ˜1M cells are harvested for each condition, along with an unedited ‘wild-type’ control, and gDNA is extracted using a ChIP lysis buffer (final concentration 1% SDS, 10 mM EDTA, 50 mM Tris-HCL, pH 7.5). The cells are resuspended in the lysis buffer and heated at 65° C. for 10 minutes. 2 μl of RNase Cocktail is then added to each tube of lysate and incubated at 37° C. for 30 minutes. Finally the lysate is treated with 10 μl of NEB Proteinase K and incubated at 65° C. for 2 hours before the enzymes are heat inactivated at 95° C. for 20 minutes

Following the gDNA extraction, the lysate from the cells are Ampure SPRI cleaned at 0.7× and PCR amplified for the region of interest where the editing occurred. The lysate is split into 2 PCR reps, each containing ˜500,000 cells. Primers are designed for each of the specific target genomic DNA sites and handles are added to the primers as shown in the table below (Table 3). The PCR1 product is checked on a gel, SPRI cleaned (1×) and a second round of a barcoding PCR is performed (3 μl of the 50 μl elute used as template). This PCR2 product is also run on a gel, pooled together (3 μl from each well) and SPRI cleaned twice at 1×. The library is run on a MiSeq at 1× coverage to determine the percentage of total editing for the population of cells. If editing has occurred at a reasonable frequency, with each variant present at >0.5% of the total population of cells, then the protocol proceeds with the RNA-FlowFISH protocol

TABLE 3
Primer sequences for genotyping the regions of interest and creating a
barcoded library

| | | | Forward/ | |
|---|---|---|---|---|
| Step | Gene | Region | Reverse | DNA Sequence |
| Genotyping | PPIF | Promoter | Forward | CACGACGCTCTTCCGATCTAACATGGAA |
| (PCR1) | | | | CCAGCAACCT (SEQ ID NO: 13) |
| | | | Reverse | GACGTGTGCTCTTCCGATCTGGGAGAAC |
| | | | | ACAGAACTCAAACT (SEQ ID NO: 14) |
| | | Enhancer | Forward | CACGACGCTCTTCCGATCTCTGCGTGCA |
| | | | | TCTTTGCTCTG (SEQ ID NO: 15) |
| | | | Reverse | GACGTGTGCTCTTCCGATCTAACTGCTG |
| | | | | CCTCATCACCTC (SEQ ID NO: 16) |
| | | Negative | Forward | CACGACGCTCTTCCGATCTCATTCCTCC |
| | | control | | AGCCTCTTGTATG (SEQ ID NO: 17) |
| | | region | Reverse | GACGTGTGCTCTTCCGATCTCACACCAT |
| | | | | CCTCGGTCTTTG (SEQ ID NO: 18) |
| | | 5′ splice site | Forward | CACGACGCTCTTCCGATCT |
| | | | | GCTCGTGTACCTGGACGTG (SEQ ID |
| | | | | NO: 19) |
| | | | Reverse | GACGTGTGCTCTTCCGATCT |
| | | | | GCCGGAAGAAATCATTCTGA (SEQ ID |
| | | | | NO: 20) |

| | | |
|---|---|---|
| Barcoding (Example only) | Forward | AATGATACGGCGACCACCGAGATCTAC |
| (PCR2) | | ACACTGTTTCCCTACACGACGCTCTTCC |
| | | GATCT (SEQ ID NO: 21) |
| | Reverse | CAAGCAGAAGACGGCATACGAGATCCT |
| | | GGTAGGTGACTGGAGTTCAGACGTGTG |
| | | CTCTTCCGATCT (SEQ ID NO: 22) |

### G. Removing Doxycycline

After confirming the successful introduction of variants into the THP-1 PE2 cells using the prime editing technology, doxycycline was removed from the media and the cells were cultured for another week in RPMI (10% hiFBS, 100 PS, 2 mM L-Glutamine) with 0.3 μg/mL of maintenance puromycin. This is to ensure that the prime editing machinery is no longer expressed and that the presence of the prime editing machinery binding to the target sites is not the reason for the changes in gene expression which are detected

### H. Performing RNA FlowFISH and FACS

### 1. Prime-Flow

RNA FlowFISH is performed on the pooled prime-edited cells after at least one week without doxycycline treatment. Approximately 12-15 M cells are harvested per FlowFISH tube. Experimentally, from each ‘biological replicate’ from the initial spinfection, between two and ten FlowFISH replicate tubes were set up. Additionally, two FlowFISH tubes were set up as ‘unstained’ controls (i.e., they contain no probe-sets and act as a control while drawing the gates during FACS).

The FlowFISH protocol is performed by using 12-15 million cells per reaction and seven washes with 40° C. wash buffer following the staining protocol. Each sample was stained for the gene of interest with an Alexa Fluor 647 (AF647, ‘Type 1’) probe set and against the positive control housekeeping gene (RPL13A) with Alexa Fluor 488 (AF488, ‘Type 4’). The PrimeFlow tubes are described in Table 4.

TABLE 4
PrimeFlow probe-sets used for the Variant-FlowFISH assay

| Gene | Type | Catalog Number (ThermoFisher) |
|---|---|---|
| PPIF | 1 | VA1-3000735-PF |
| RPL13A | 4 | VA4-13187-PF |

### 2. FACS

Following the preparation of the samples via RNA FlowFISH, the samples are prepared for FACS. For example, with twelve FlowFISH tubes per ‘Biological Replicate’, ‘FlowFISH replicate’ tubes 1-3, 4-6, 7-9 and 10-12 are pooled together, to make FlowFISH replicates 1-4, respectively. Approximately 200,000 cells are collected from each tube as a genotyping control to understand the variants present within the FlowFISH samples or ‘input’ for later analysis.

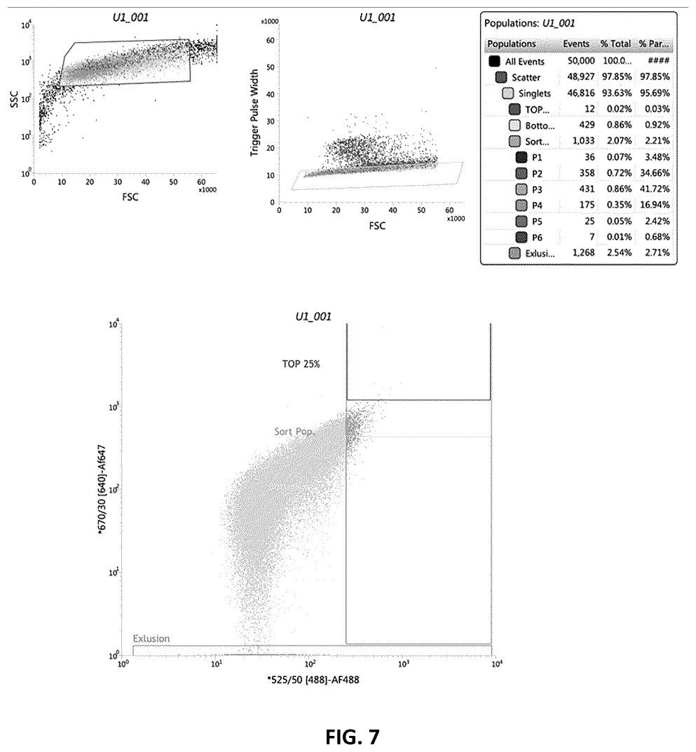

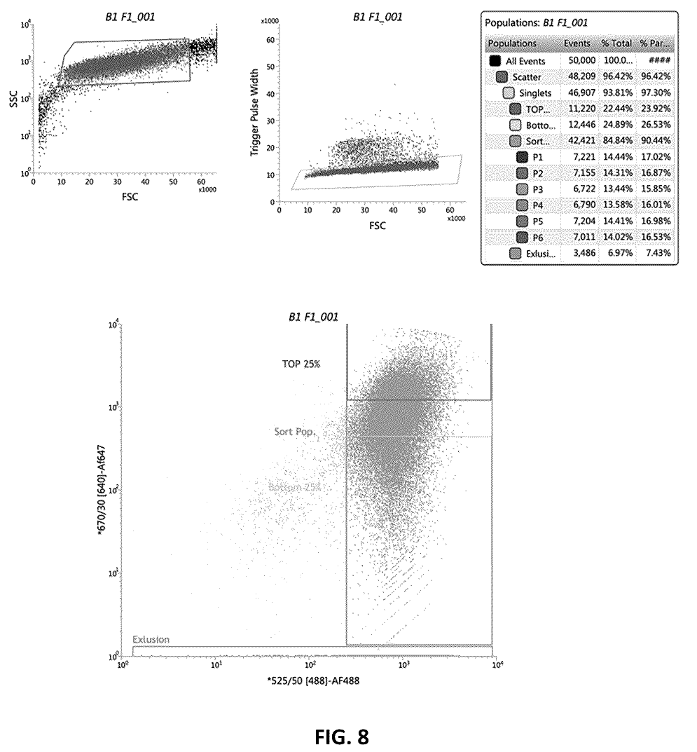

Samples are sorted on the Influx FACS sorter using the gating strategy shown in FIGS. 7-9. Briefly, the first two gates in FIGS. 7 and 8 are standard and select cells and then single cells. The third plot in FIGS. 7 and 8 has compensated *525/50 [488]-AF488 on the x axis and *670/30 [640]-AF647 on the y-axis. The compensation value is added to this population because there is a relationship between the total amount of RNA (normalized by the RPL13A reference control) and the expression of the gene of interest.

The unstained cells are loaded onto the sorter first (FIG. 7) and a gate is drawn as to where the stained or ‘sort’ population should be. Up to 10% of the unstained cells are allowed to be in this gate but the lower the amount, the better. Next, the stained sample is loaded on the FACS sorter and analysis confirms that this sample falls within the ‘sort’ population gate (FIG. 8) Some additional gates are drawn as controls during the sort. An exclusion zone is drawn at the bottom of the plot and can contain up to 10% of the cells. As a check to confirm that the compensation value is approximately comparable between each sort, two additional gates are drawn covering the top 25% of cells and the bottom 25% of cells of the ‘sort’ population. A goal is to have the mean of the sorted population in the 525/50 [488] channel to be within 10% of the mean for the top 25% and the bottom 25% populations. For example, if the mean is 1000 for the sorted population, then the mean for the top 25% population should be ˜1100 and the mean for the bottom 25% population should be ˜900. While this may not always be possible the compensation values can be adjusted to obtain values as close as possible to these parameters.

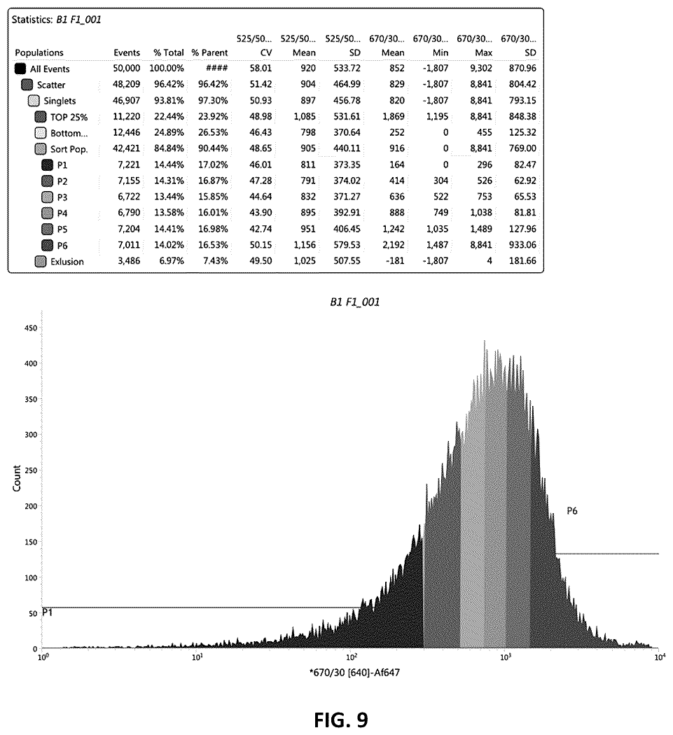

The plot of FIG. 9 is a histogram which has the compensated *670/30 [640]-AF647 channel on the x-axis and count data on the y-axis. This histogram is then divided into six ‘bins’ or ‘sets’ spanning the entire distribution (˜16.6% of the sorted population in each bin). These six bins are termed bins A, B, C, D, E and F. With bin A containing the cells having the lowest expression of the gene of interest and bin F containing the cells having the highest expression levels of the gene of interest. The FlowFISH sample cells are sorted into each of these bins and collected. Additionally, the number of cells collected in each bin, and the mean, minimum and maximum value for each of the bins, is recorded as this data can be used for reconstructing the distribution of the population and determining the effects of each of the variants. This methodology has also been validated for other numbers of sets or bins, e.g., with a 4-bin sorting approach.

### I. Preparing Sequencing Library

Following the FACS sort of the FlowFISH samples, the genomic DNA from each of the bins and input samples is harvested. Approximately 5M cells are collected in each bin so the gDNA extraction process is scaled accordingly. Those 5M cells are split equally into 5 wells of a 96-well plate and the gDNA extraction process is applied to the samples.

Additionally, a library is prepped for Illumina sequencing with two rounds of PCR. The first of these is to amplify the region of interest from the genome, and the second is to add Illumina sequencing handles compatible with Next-Seq sequencing platforms.

### J. Analyzing Variant FlowFISH Screens

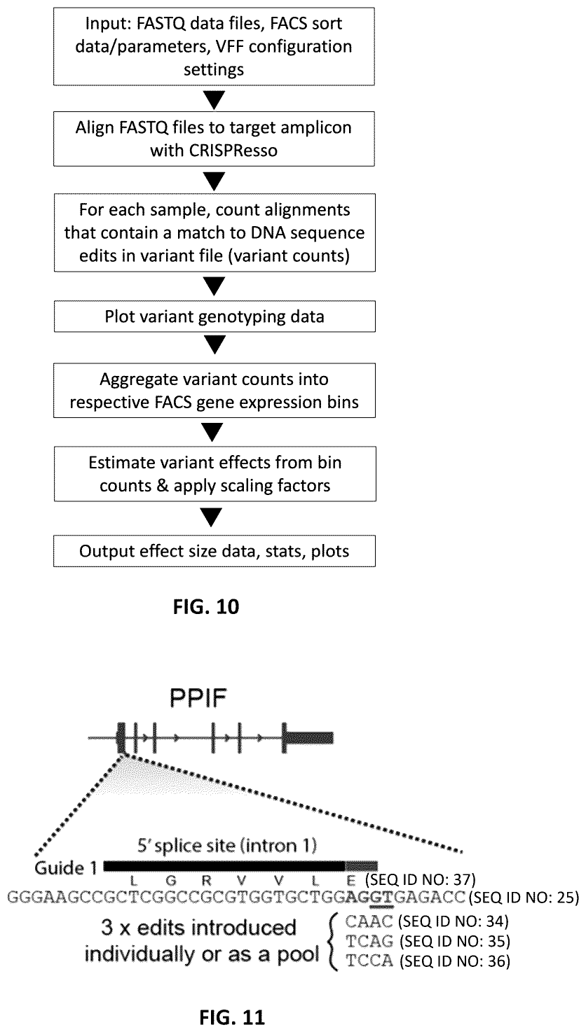

To determine the effects of each edit on fluorescence, a maximum likelihood estimation method was applied to the normalized distribution of sequencing reads for each edit across FACS bins. The workflow for our Variant-FlowFISH computational analysis is summarized in FIG. 10. First, raw sequencing reads were demultiplexed using the bcl2fastq program (Illumina) and CRISPResso2 was used to align reads with a minimum average quality score of 20 (phred 33) to a reference amplicon sequence. For the filtered and aligned reads in each sample, the number of unedited reference reads, as well as the number of reads containing each edit specified in a variant list file, were counted. The variant list file contains a “Mapping Sequence” column, which is a sequence containing the expected variant as well as 5-bp buffer on each side of the variant. If a read exactly contained a variant's mapping sequence, it was added to the variant's read count.

For each sample, which corresponds to a FACS bin of a PCR replicate, a count was made of how many of the reads correspond to an introduced variant and how many are wild type. If the wild-type reference allele mapping sequence was provided, then the wild-type count was obtained from matching reads to the provided sequence. However, for long amplicons, there are more likely to be PCR and sequencing errors in the reference sequence. In this case, if sequences are labeled as reference only if they are an exact match, the reference allele read counts will likely be underrepresented. To address this issue, an option was added to infer which reads are unedited reference reads by using reads unmatched to a variant, and applying an error threshold to approximately match each read to the wild-type sequence.

The read counts were aggregated across PCR replicates to obtain a total count for unedited reference and designed edit sequences in each FACS bin. These aggregated counts were normalized so that the sum of reference and variant alleles in each sample equaled the total number of cells sorted into that FACS bin (i.e., to account for differences between the number of sequencing reads and the number of cells in a given bin).

The limited-memory Broyden-Fletcher-Goldfarb-Shanno algorithm maximum-likelihood method in the R stats4 package was then used to fit the read counts in each fluorescence bin to the log-normal distribution that would most probably have produced the observed counts in the bins. To calculate effect sizes, the percent difference between the mean of the log-normal fit for each edit and the mean of the log-normal fit for the wild-type reference sequence was determined. Two scaling factors were applied to these effect sizes: one to account for background signal in the RNA FISH assay, and one to account for heterozygous editing events during prime editing. First, the difference between CRISPRi-FlowFISH TSS guide knockdown and qPCR effect size was used to generate a linear scaling factor applied to effect sizes, which accounts for FlowFISH signal from non-specific RNA FISH probe binding (this is based on the observation that promoter CRISPRi typically shows 80-90% knockdown by qPCR). Next, the editing efficiency of each designed edit in the pool (edit frequency in pool * total number of edits) was calculated and applied to the stable allele frequency equation to obtain an estimate of the fraction of cells receiving a specific edit on one or both alleles. A derived function was then applied to the effect sizes to account for the estimated allelic editing rate of each designed edit.

$$1 + y = \frac{( 1 + x ) \frac{{p}^{2}}{p ( 1 - p ) + {p}^{2}} + ( 1 + \frac{x}{2} ) \frac{p ( 1 - p )}{p ( 1 - p ) + {p}^{2}}}{\frac{{( 1 - p )}^{2}}{p ( 1 - p ) + {( 1 - p )}^{2}} + ( 1 + \frac{x}{2} ) \frac{p ( 1 - p )}{p ( 1 - p ) + {( 1 - p )}^{2}}}$$

where x equals the effect size of the homozygous variant, y equals the measured variant effect through Variant-FlowFISH, and p equals the allele frequency of the introduced variant. The actual effect size output by Variant-FlowFISH is 1+x, as the effect size was normalized such that the wild type “effect” is equal to 1 (x=0, no change in expression), with effect sizes larger than 1 indicating a positive effect on expression, effect sizes smaller than 1 indicating a negative effect on expression, and an effect size of 0 indicating no expression.

Several simplifying assumptions were made when setting up the above equation. Firstly, it was assumed that both alleles in the diploid cell are independently edited, such that the distribution of edited and unedited alleles would follow p\hat{ }2+2pq+q\hat{ }2=1. Secondly, it was assumed that the effect of a variant edited heterozygously is half that of the homozygously edited variant. Finally, it was assumed that the wild-type, homozygous edited, and heterozygously edited cells are normally distributed in log space during the FACS sort and that the homozygous and heterozygous cell distributions have the same variance.

The equation states that the measured variant effect from Variant-FlowFISH is equal to the homozygous effect size multiplied by the proportion of the cells that are homozygously edited plus the heterozygous effect size multiplied by half of the proportion of cells that are heterozygously edited, divided by the reference effect size (1) multiplied by the proportion of cells that are wild type (unedited) plus the heterozygous effect multiplied by the other half of the heterozygously edited cells.

When solving for x (the change in effect size from reference), a simplified form of the above equation can be obtained:

$$x = \frac{2 y}{1 - py}$$

If it is considered that the wild-type effect size is not impacted by heterozygous editing, as usually a majority of the cells are wild-type, then the denominator of the first equation can be set to 1. That change leads to this simplification:

$$x = \frac{2 y}{1 + p}$$

This provides some intuition that if there is almost no editing (p˜=0), the actual homozygous effect should be two times the measured effect. If almost all the alleles are edited (p˜=1), the homozygous effect should be very close to the measured effect.

### Example 2. Pooled Measurements of Effects of Sequence Edits on Gene Expression

This example describes an application of the provided high-throughput technology designated Variant-FlowFISH, which has the capability to edit hundreds of variants into the genome via CRISPR prime-editing and measure the effect of these variants on gene expression in parallel, via using an RNA FlowFISH (fluorescent in situ hybridization) flow cytometry based screening strategy (FIG. 1) (Fulco et al., Nat. Genet. 51, (2019): 1664). Briefly, a ‘pooled prime-editing’ approach was used, where prime-editing is applied to introduce up to 100+ variants at a single endogenous target loci, such as an enhancer or promoter. The desired edits are introduced into a population of cells containing the doxycycline inducible Cas9 nickase-reverse transcriptase (nCas9-RT) from the PE2 version of prime editing (Anzalone et al., Nature 576, (2019): 149), via lentivirus containing prime-editing guide RNAs (pegRNAs). Successfully infected cells are selected via puromycin resistance and subsequently the prime-editing machinery is induced through doxycycline treatment of the cells (here, for 14 days), wherein the editing efficiency was observed to increases with time (FIG. 31). Next, doxycycline is removed for at least 7 days, to ensure that any changes observed for gene expression were due to the introduced variants and not due the presence of the prime-editing machinery binding to the CRE. To measure the effect of each variant on the expression of the gene of interest, the method in this example includes (i) taking the population of edited cells and performing RNA FlowFISH for the gene of interest, and (ii) sorting the cells into 4-6 bins based on their expression levels of this gene, via fluorescent activated cell sorting (FACS). Alternatively, it is also possible to adopt protein-antibody fluorescent conjugate based measurements of gene expression instead of RNA-FISH during this cell sorting process. The method of this example further includes (iii) extracting genomic DNA from the cells and PCR-amplifying the edited site, using high-throughput sequencing to determine the frequency of the variants across each of the bins.

A mathematical approach and computational pipeline was developed to use these data to estimate the quantitative effect of each edit, considering editing efficiency and cell ploidy. Because prime editing is not 100% efficient, some cells will carry homozygous edits and some will carry heterozygous edits, each of which will show different levels of expression from RNA FISH. Accordingly, the provided computational pipeline infers the effects of edits on gene expression by adjusting a previous maximum likelihood estimation procedure (Nasser et al., Nature 593, (2021): 238) to account for a distribution of genotypes in the population of cells carrying 0, 1, or 2 copies of the intended edit. Notably, this estimation procedure assumes that, in diploid cells with two alleles of the targeted site, (i) the editing of each allele in a cell is independent of the other, and (ii) a single cell does not receive two different edits, which holds to a sufficient extent for CRISPR prime editing due to its precision in installing the intended edit, but is not generally the case for alternative editing methods such as CRISPR HDR or base editing that introduces frequent unintended edits at the targeted site. In particular, regarding the latter assumption, the introduction of unintended edits can create both false positives and false negatives.

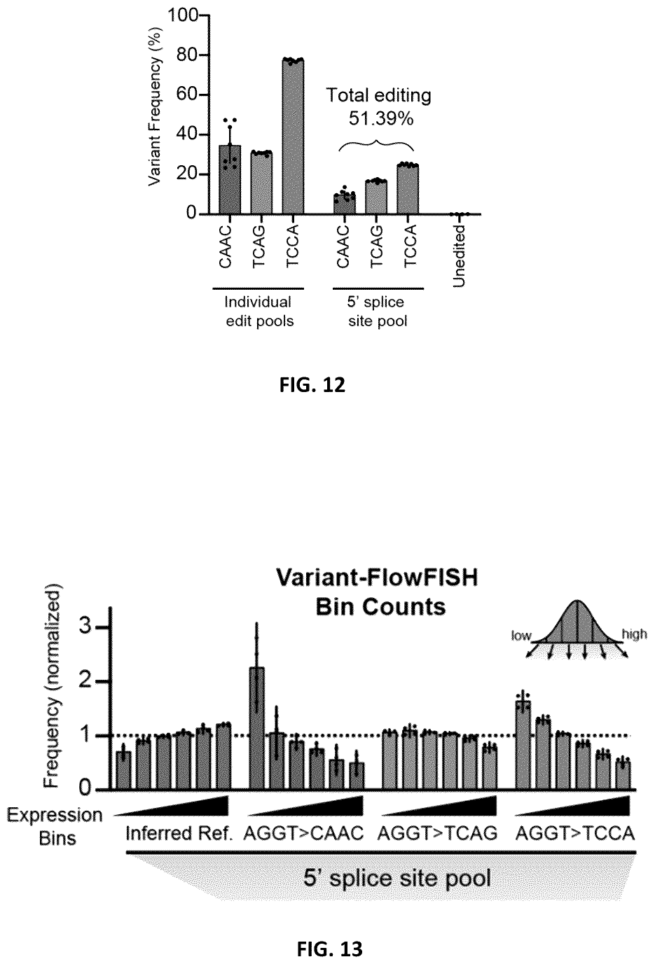

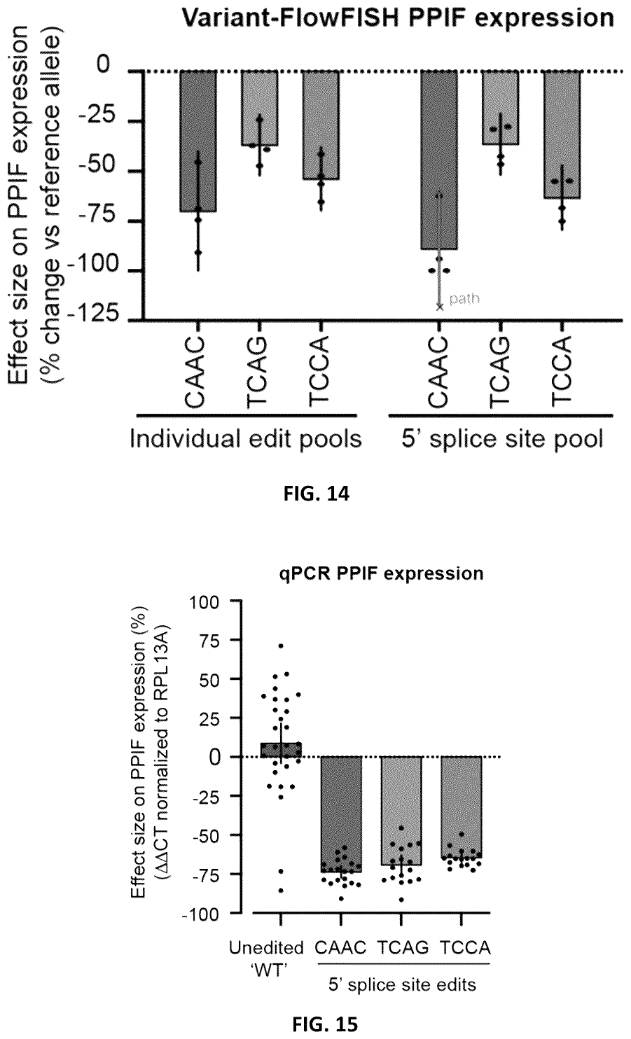

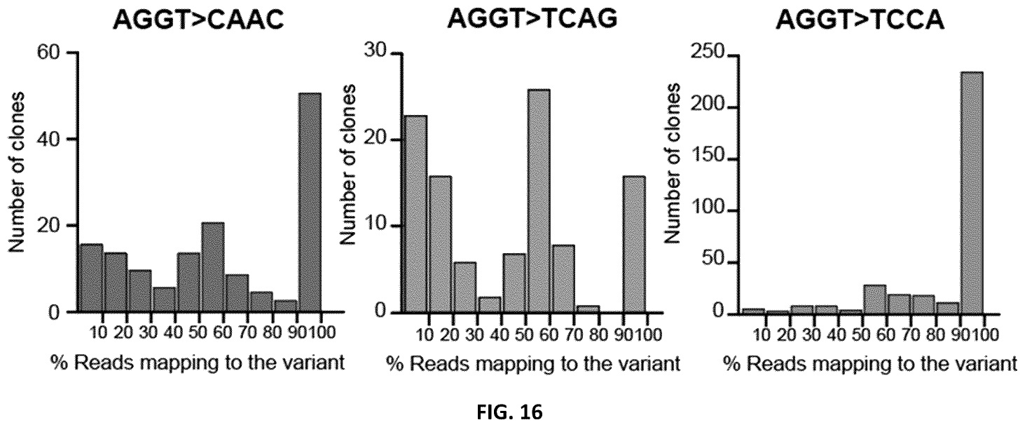

To demonstrate the provided technology, i.e., the Variant-FlowFISH technology, a proof-of-concept study was designed to introduce sequence edits that should strongly reduce expression of a target gene of interest, PPIF (FIG. 11). The PPIF gene is involved in tuning the mitochondrial membrane potential in macrophages, and previous studies used KRAB-dCas9 (CRISPRi) combined with RNA FlowFISH to identify regulatory elements that control PPIF expression in several immune cell lines (Nasser et al., Nature 593, (2021): 238). Three edits were designed to disrupt the ‘GT’ splice donor at the first 5′ splice site (AGGT>CAAC, AGGT>TCAG, and AGGT>TCCA), which was expected to lead to strong decreases in expression resulting from aberrant splicing and nonsense mediated decay. One pegRNA was designed to introduce each of these three sequence edits, and populations of THP-1 PE2 cells were transduced either with each pegRNA individually or with a pool of all three pegRNAs. The edited site was sequenced and an editing rate of 34, 31, and 77% was observed for the populations of cells that received a single pegRNA, with a total editing rate of 51% observed in the population of cells that received all three (FIG. 12). Variant-FlowFISH was performed on these populations of cells and the frequencies of the variants across the six bins after FACS sorting were determined via sequencing the alleles in each of the bins (FIG. 13). This frequency distribution was used to determine the effect of each of the variants using the maximum likelihood estimation procedure and a 36.94-69.89% decrease in PPIF expression was detected for the single edits and a 36.43-89.15% decrease of PPIF gene expression was detected for the ‘mini-pool’ (FIG. 14). To validate the Variant-FlowFISH technology, homozygous clonal cell populations were derived and genotyped (FIG. 16) and qPCR was performed for each of the 5′ splice site edits, resulting in detection of a 73.70%, 69.26% and 64.62% decrease for each of the AGGT>CAAC, AGGT>TCAG, and AGGT>TCCA edits, respectively (FIG. 15). Therefore, these results show that the Variant-FlowFISH technology can accurately detect the effects of sequence edits on gene expression and can be multiplexed to study multiple edits in a pooled manner.

### Example 3. Tiling Mutagenesis of Regulatory Elements in the Human Genome

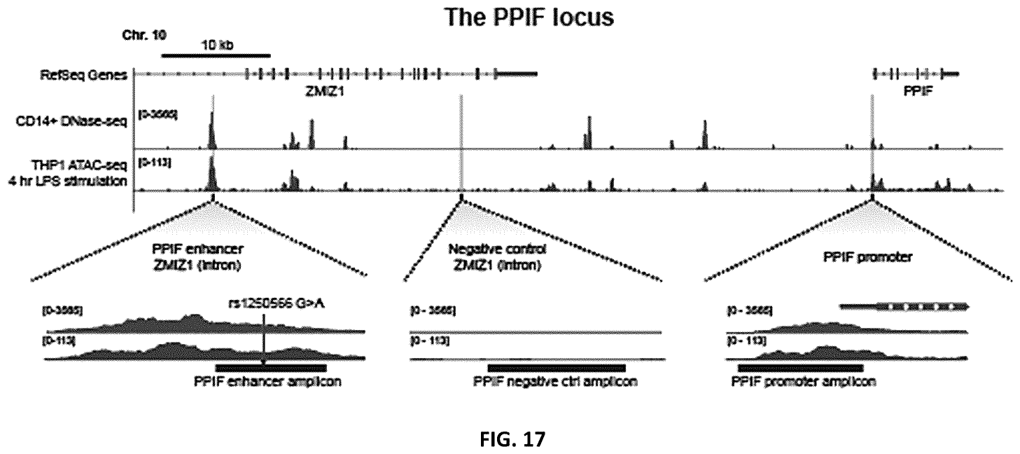

A next study explored the utility of Variant-FlowFISH in mapping the functions of regulatory DNA sequences in their endogenous locations in the genome through high-throughput tiled mutagenesis. To do so, the PPIF promoter, a distal enhancer of PPIF, and a negative control region were selected, and experiments were designed to systematically identify sequence motifs important for regulating PPIF gene expression (FIG. 17). This distal enhancer which contributes to PPIF expression was recently identified in an intron of the neighboring gene, ZMIZ1, and it was shown that perturbing this region by CRISPRi-FlowFISH reduced PPIF expression by ˜40% (Nasser et al., Nature 593, (2021): 238). Furthermore, this enhancer contains a single-nucleotide polymorphism (SNP) linked to inflammatory bowel disease (IBD). The negative control region was randomly selected at this loci, based on a lack of chromatin accessibility, as determined by DNase-seq and ATAC-seq data in relevant cell types (FIG. 17).

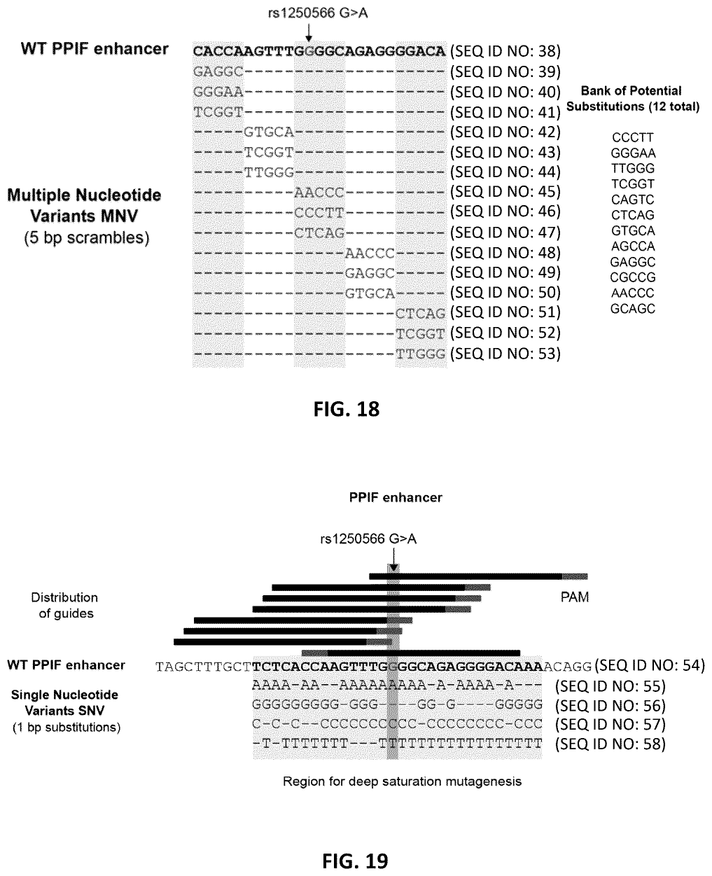

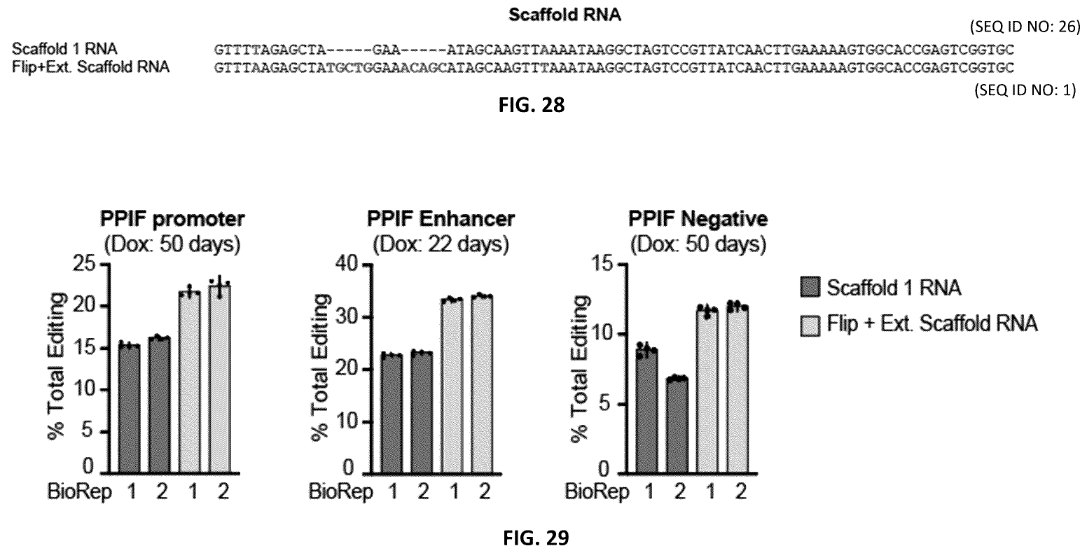

With the aim of introducing drastic changes to the DNA sequence at the PPIF enhancer, proximal promoter, and negative control region, 5-bp multi-nucleotide variants (MNV) or substitutions across these three regions, were designed and systematically tiled (FIG. 18). The MNVs contained a GC content >50% and were positioned end to end, with each position receiving three different sequences from a bank of 12 possible substitutions. This was to account for the chance of any particular MNV altering PPIF gene expression, by creating a de novo site for a new transcription factor which does not normally act through that region. The experiments were performed in pools for each of the three different sites, with over 100 prime-editing guide RNAs (pegRNAs) being designed for each region: 105 pegRNAs for the PPIF enhancer, 132 pegRNAs for the PPIF promoter and 126 pegRNAs for the PPIF negative control region. Additionally, in order to dissect whether the SNP located within the PPIF enhancer is important in controlling PPIF gene expression, a pool of 90 pegRNAs was designed to perform saturation mutagenesis and introduce single-nucleotide variants (SNV) across a 30-bp region of the PPIF enhancer, centered over the IBD SNP (FIG. 19). In terms of pegRNA design, spacers were selected no more than 50 bp away from the nick site, the reverse transcriptase template (RT-template) was between 15 and 50 bp long, the primer-binding site (PBS) was 11 bp long, and an optimized flip+extension scaffold sequence was utilized as it increased editing on average by a fold change of 1.46 (FIGS. 26-29).

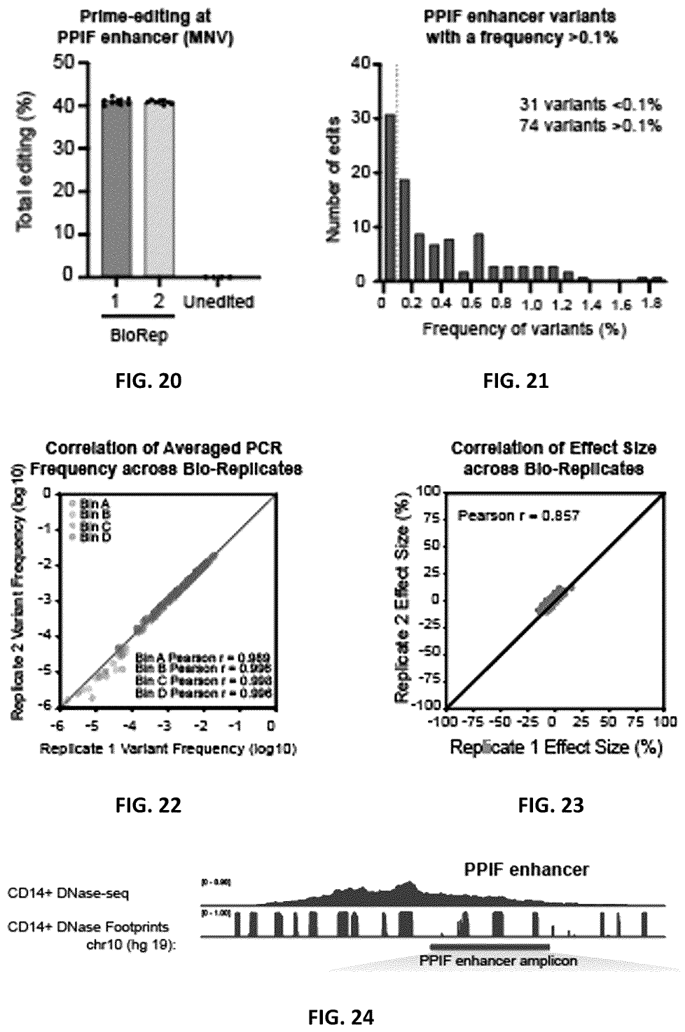

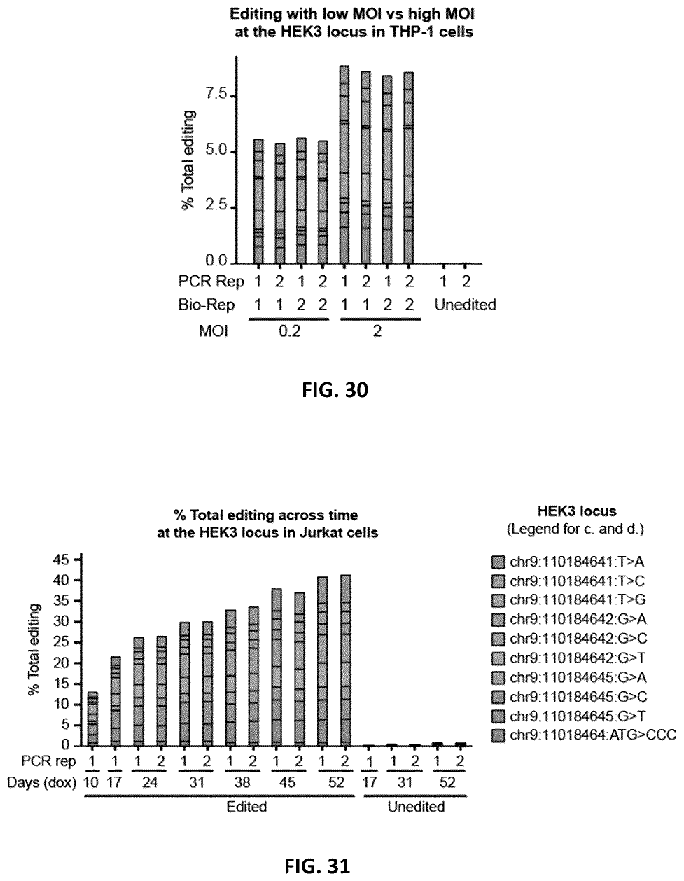

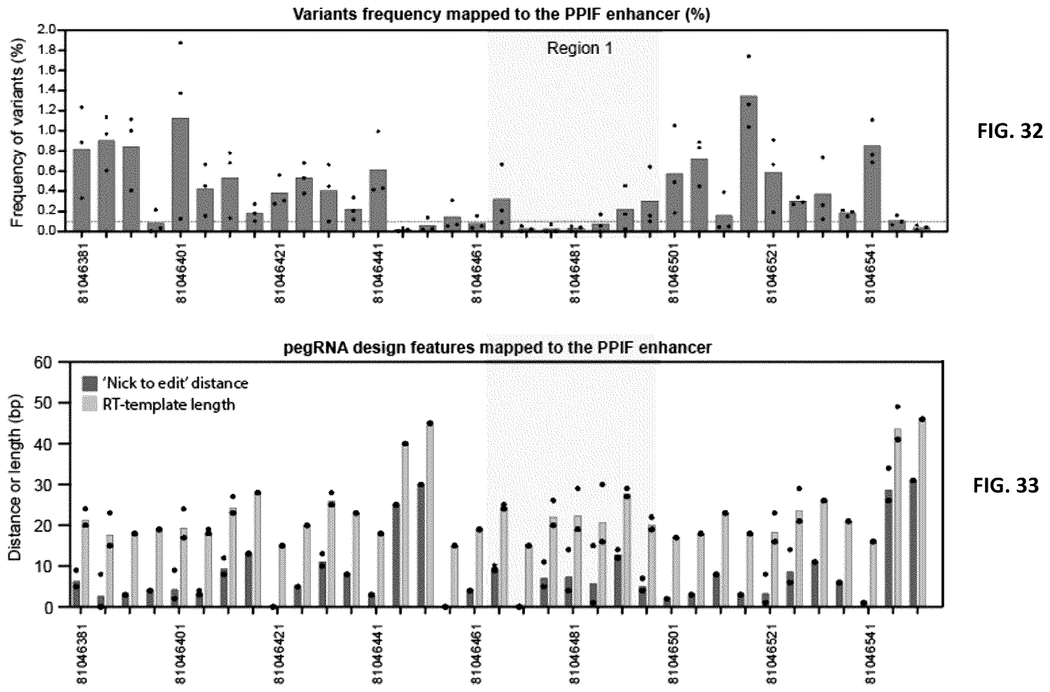

The pools of 100+ pegRNAs for each of these regions within the PPIF locus were introduced into the THP-1 doxycycline-inducible PE2 cells line via lentivirus, using a multiplicity of infection (MOI) of 2. Since not every pegRNA is efficient at introducing the desired edit, preliminary experiments showed that the percentage of total editing increased by increasing the MOI (0.2 vs. 2) and by also increasing the amount of time the PE2 machinery is induced by a doxycycline treatment (FIGS. 30 and 31). After 14 days of doxycycline induction of the PE2 system, a total editing rate of 40.92% was observed for the MNV substitutions at the PPIF enhancer (FIG. 20). However, not every desired MNV was successfully introduced at the target loci, with 29.5% (31 out of 105) of the pegRNA edits being detected at a frequency <0.1% at the PPIF enhancer (FIG. 21). While many of the edits at this low frequency (<0.1%) fall within the same region of the PPIF enhancer, there does not seem to be a clear difference between pegRNA design features between regions which did edit at a higher efficiency, compared to those which did not, in terms of the ‘nick to edit’ distance for the pegRNA spacer or the RT-template length (FIGS. 32 and 33).

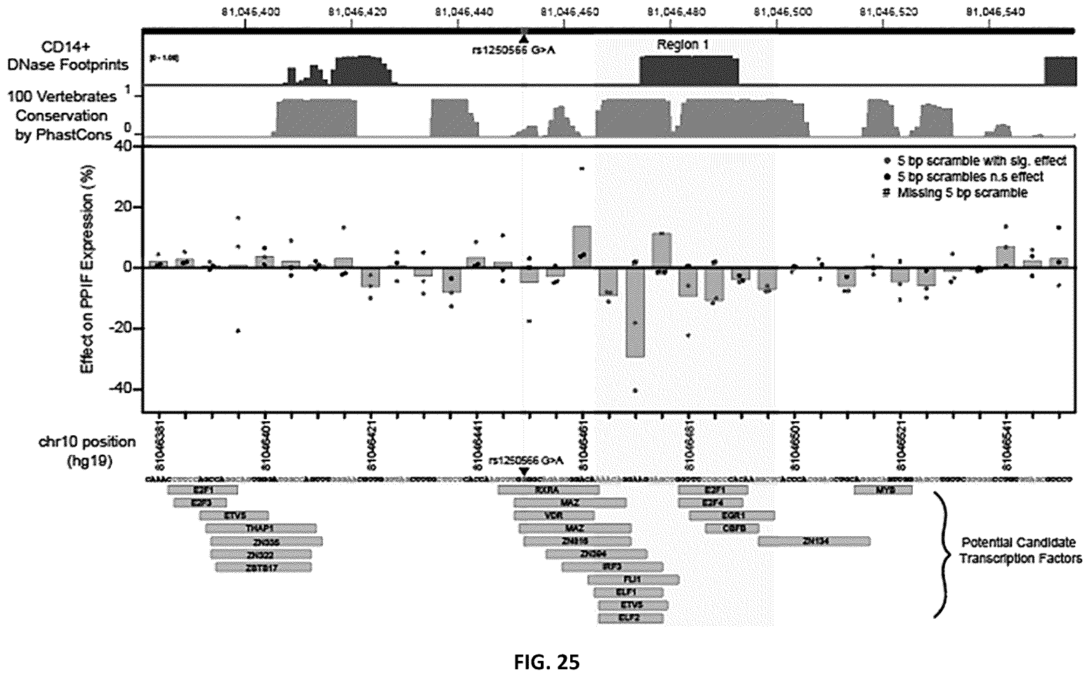

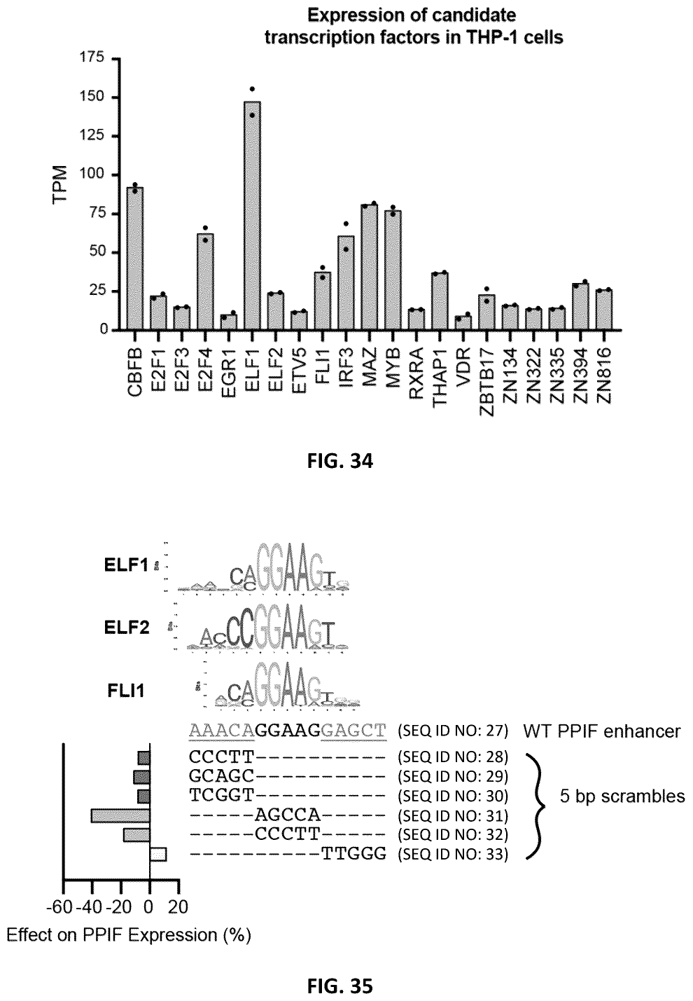

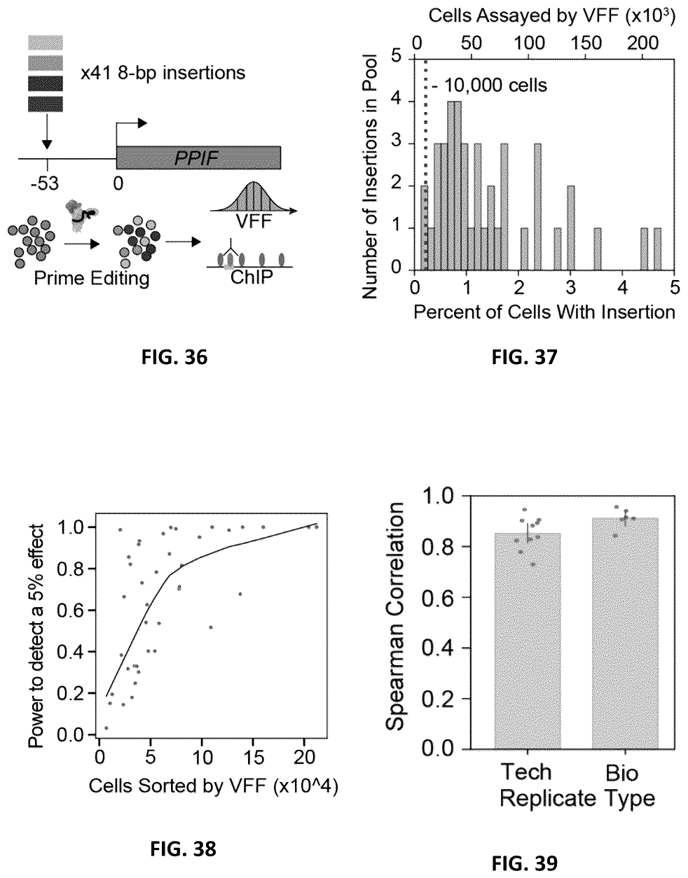

Next, Variant-FlowFISH was performed for the pool of cells containing 100+ MNV edits at the PPIF enhancer region, to determine the effect of each of these edits on PPIF gene expression. After sequencing the alleles in each of the bins following RNA FISH and FACS, a high correlation was observed for the frequency of the variants at a PCR level, across bio-replicates (Pearson r=0.989-0.998, FIG. 22). The maximum likelihood estimation procedure was applied to determine the effect size of each of our MNVs, producing a Pearson r=0.857 correlation between the biological replicates (FIG. 23). Mapping the MNVs introduced into the PPIF enhancer back to the original DNA sequence revealed 47.6% (50/105) of the scrambles introduced were shown to have a significant effect on PPIF gene expression, and using Variant-FlowFISH technology, changes of PPIF expression ranging from 1.25-40.38% could be detected (FIG. 25). This analysis revealed a 35-bp region of particular interest, termed ‘Region 1’, where 14 edits caused a significant decrease of PPIF gene expression, ranging from −4.6% to −40.4% (FIG. 25). The PPIF enhancer DNA sequence was aligned with CD14+ DNase-seq data, DNase Footprints, and conservation tracks from vertebrates, which highlighted that the chromatin within the PPIF enhancer region is accessible and contains footprints which align with regions which are highly conserved within vertebrates (FIG. 25). Finally, to identify potential candidate transcription factors binding to ‘Region 1’ of the PPIF enhancer, the Find Individual Motif Occurrences (FIMO) tool was used (FIG. 25), together with RNA-seq data from THP-1 cells (FIG. 34), to highlight several transcription factors, including members of the ETS transcription factor family (FIG. 35), which could be binding the PPIF enhancer to control PPIF gene expression in THP-1 cells.

Therefore, this analysis at the PPIF enhancer shows that it is possible to introduce ˜100 edits within a single pooled experiment, to detect changes in gene expression as low as 1.25%, and to map key motifs within cis-regulatory elements, thereby identifying potential candidate transcription factors which could be controlling expression of the PPIF gene through binding to the PPIF enhancer.

### Example 4. Rewriting of Transcription Factor Binding Sites

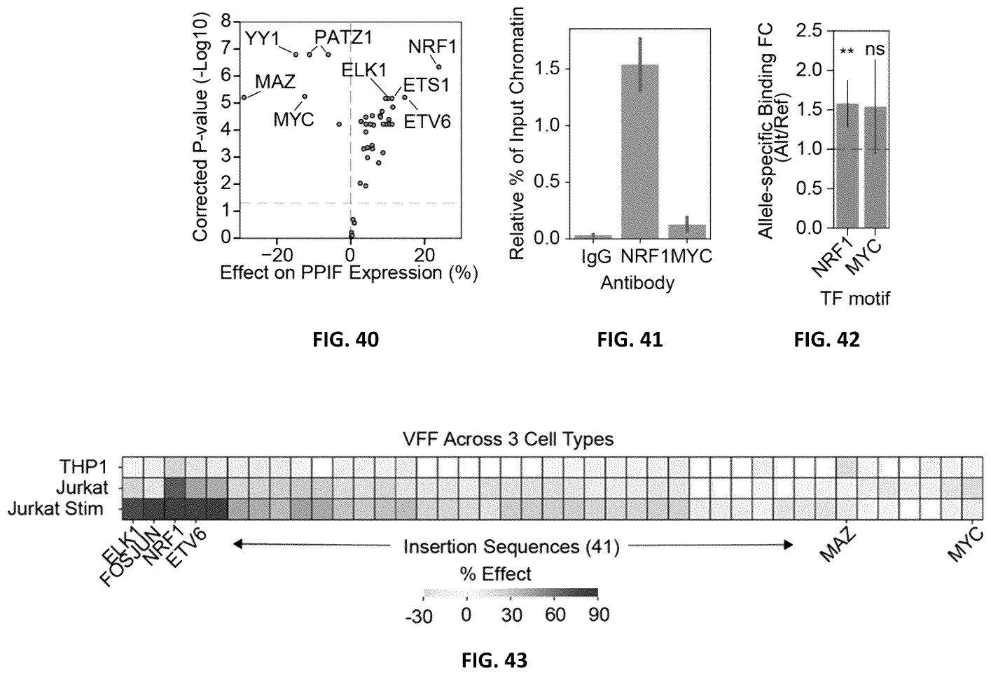

A next study considered the utility of Variant-FlowFISH (VFF) to characterize the effects of engineered sequence insertions at a single endogenous locus in native cellular context. To explore this application, a site with high editing efficiency was identified 53 bp upstream of the PPIF TSS and a library of pegRNAs was designed for insertion of synthetic 8-bp sequences into this site (FIG. 30). A total of 41 insertions we designed, including 8-bp sequences predicted to create new transcription factor motifs, modify or break the existing SP1 motif overlapping the insertion site, or have minimal effect on endogenous motifs in the PPIF promoter. These insertions were deployed in THP-1 monocytes and their effect on PPIF expression was evaluated using VFF. Pooled prime editing correctly integrated the insertions into the PPIF promoter at an average frequency of 1.46% per edit with a range of 0.15-4.65%, and >10,000 cells were assayed for 40 out of 41 edits (FIG. 37). At this coverage, >80% power was available to detect an effect size of 10% for all 40 edits at a cell coverage threshold of 10,000, and a subset of 18 edits in this group had >80% power to detect an effect size of 5% (FIG. 38). The observed effect sizes for each insertion were highly correlated at both technical (rₛ=0.85) and biological (rₛ=0.90) replicate levels, and 36 out of the 41 edits (88%) significantly altered PPIF expression in THP1 monocytes (FIGS. 39 and 40).

The pool of synthetic 8-bp insertions induced a wide range of effect sizes on PPIF expression, encompassing edits that either significantly increased or decreased gene expression relative to the wild-type promoter (FIG. 40). The strongest activating edit in the dataset corresponded to an insertion that is predicted to create a motif for the cell-essential TF NRF1 (+24% effect; P=4.6×10⁻⁷, t-test BH corrected), followed by motifs for ETS family factors (ELK1, ETS1, GABPA). Consistent with the observation that these motifs can upregulate expression when inserted into a native promoter, previous studies have found them to be highly enriched in strong enhancers and promoters and demonstrated their ability to boost expression across a series of sequence and cell types using massively parallel reporter assays. The sequence insertion that led to the strongest decrease in gene expression in the dataset corresponded to a motif for the insulating TF MAZ (−29%; P=6.2×10⁻⁶, t-test BH corrected), followed by motifs for YY1 and MYC. Notably, both YY1 and MYC have been reported to reduce expression by interfering with the binding and activating functions of TFs such as SP1 and NF-Y, and motifs for both of these TFs exist in close proximity to the insertion site for the 8-mer pool. Furthermore, NF-Y has been shown to recruit core components of the transcription preinitiation complex (PIC) such as TFIID, and the positioning of this motif in the TATA-less PPIF promoter suggests that it may be important for guiding recruitment and positioning of transcriptional machinery.

To verify that the observed effects are associated with recruitment of the TFs that were predicted to bind the synthetic sequences introduced in this screen, a chromatin immunoprecipitation (ChIP) experiment was performed to probe for binding of NRF1 and MYC to WT and edited PPIF promoter sequences. ChiP-qPCR was first performed on wild-type cells to establish baseline binding of NRF1 and MYC at the PPIF promoter, which encodes an NRF1 motif 42 bp downstream of the insertion site but does not contain any high-confidence MYC motifs (FIG. 41). As expected, ChIP signal was only observed for NRF1. To investigate whether insertion of synthetic motifs for these TFs influences their binding frequencies at the promoter under native chromatin context, prime editing was used to insert the 8-mer sequence that creates MYC or NRF1 motifs into a pool of cells at the same insertion site as before and ChIP paired with amplicon sequencing was performed to quantify allele-specific TF binding. It was found that introducing the synthetic NRF1 motif into the PPIF promoter increased NRF1 binding by approximately 1.5-fold, and that MYC recruitment was increased by a similar but statistically insignificant proportion (FIG. 42).

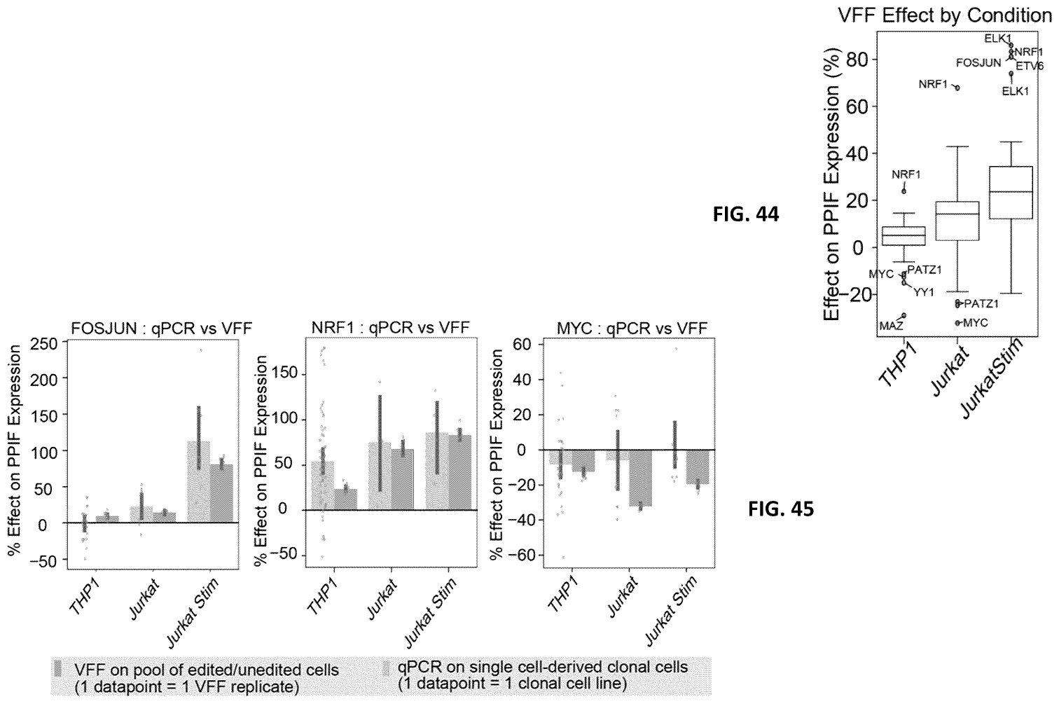

To investigate the influence of native regulatory context on the transcriptional effects of the synthetic motifs and validate the sensitivity of VFF across multiple cell types and states, the VFF screen was repeated in two additional conditions: Jurkat T immune cells with and without immune ligand stimulation. The effect sizes of each 8-mer in the screen was determined within each condition independently and then these effects were compared across conditions (FIG. 43). The effect sizes of many insertions in the screen appeared to be consistently larger in Jurkat cells than in THP1 cells, and this same trend was even more pronounced when comparing within the two Jurkat conditions (FIG. 44). Interestingly, some of the edits also showed highly condition-specific effects, such as FOSJUN (FIG. 43). The effect of this edit increased from 10% and 14% in THP1 and Jurkat T-cells (respectively) to 81% in stimulated Jurkat T-cells (6-8-fold change) and is recognized by AP-1, a critical regulator of gene expression during T-cell activation. To verify that the effect size patterns that were observed across conditions were not a technical artifact related to VFF performance in different cell types, the effects of the FOSJUN, NRF1 and MYC motif insertions were measured in clonal cell lines using qPCR (FIG. 45). Together, these results highlight the ability of VFF to investigate the quantitative relationship between non-coding regulatory DNA and cell type specific gene expression and provide several examples of how cell type-specific regulatory environments tune the transcriptional output of regulatory motifs in the genome.

### Example 5. Deep Learning Applied to Re-Engineering of Gene Expression

The results of the synthetic DNA sequence insertion experiments led to consideration of how reprogramming of non-coding regulatory DNA could be optimized to yield precise and tunable changes to cell type specific gene expression, which has potential for application to synthetic biology and gene therapy. However, iteratively screening and optimizing synthetic DNA edits that would meet criteria for these applications is expensive and time consuming to perform experimentally. To address this bottleneck, a new computational framework was designed based on MCMC-style simulated annealing and deep-learning to design and optimize sequence edits in silico.

In this computational framework, random sequence insertions ≤10 bp were initialized at pre-selected loci and 1,000 randomly sampled alterations to the sequence edit were performed (FIG. 5). These alterations were limited to insertion, deletion, or substitution of 1 nucleotide in the edit, or a 1-bp shift of the edit insertion site. The effect of the edit after each alteration was predicted using the deep learning-based sequence model, Enformer, which was recently developed to predict cell type-specific gene regulatory signals directly from DNA sequence across hundreds of cellular contexts (Avsec et al., Nat. Methods 18, (2021): 1196). The 1-bp change made to the sequence edit was accepted if it increased the predicted effect sought to be optimized. By annealing slowly, this approach can identify sequence edits that reach a near-global optimal impact on non-coding DNA sequence function as predicted by Enformer.

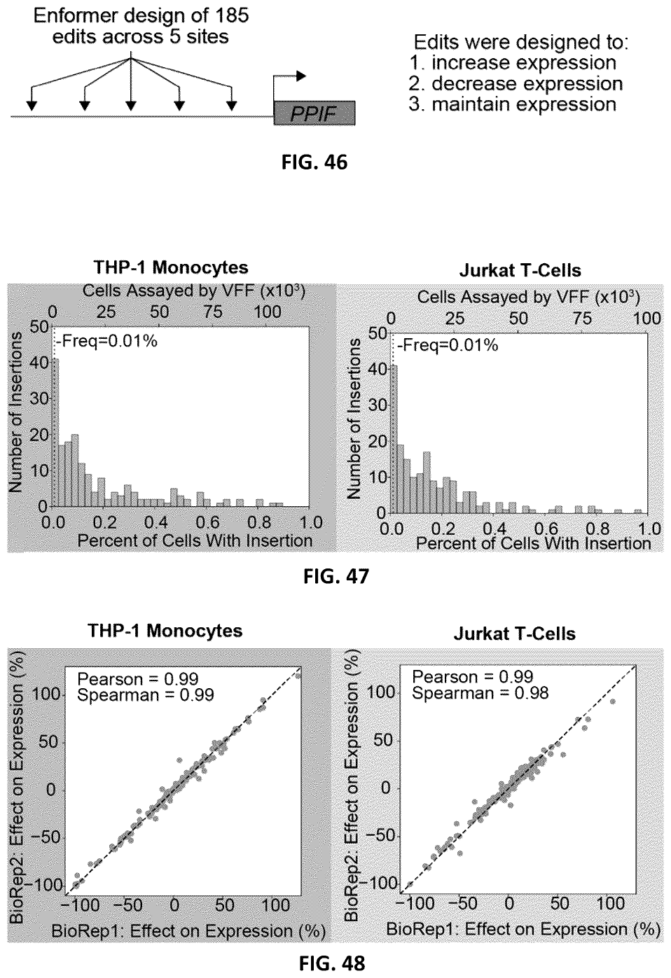

To test this new principal approach for design of synthetic DNA edits, five high-efficiency pegRNA editing sites at the PPIF promoter were selected and 185 randomly initialized sequence edits were optimized across these sites using the provided computational framework (FIG. 46). These edits were optimized to increase gene expression, decrease gene expression, or have no impact, as determined by predicted fold change in cap analysis of gene expression (CAGE) signal between the edited and wild type PPIF promoter sequence (estimated from the three Enformer output bins overlapping the TSS). Edits were also optimized to additionally maximize the absolute fold change between the THP1 and Jurkat outputs (e.g., THP1- or Jurkat-specific designs).

A library of pegRNAs encoding these edits was then cloned into a vector that adds the trimmed evopreQ1 pseudoknot sequence onto the 3′ end (Anzalone et al. Nat. Biotechnol. 40, (2022): 731) and these edits were screened in THP-1 monocyte cells and Jurkat T cells using Variant-FlowFISH. Of the 185 edits in the pool, 163 (88%) were above the filtering threshold of 0.01% frequency in both cell types. These successful edits were introduced into the PPIF promoter at an average frequency of 0.24% per edit with a range of 0.01-1.5%, and >10,000 cells were assayed for 109 of these edits in THP-1 cells, with similar results for Jurkat (average: 0.19%, range: 0.01-0.95, 108 edits >10K cells) (FIG. 47). Notably, 67 of the designed edits in this screen were insertion-deletion combinations (e.g., deleting 1 bp of endogenous DNA sequence and inserting 8 bp of synthetic sequence) and 65 of these passed the filtering threshold in both cell types, demonstrating the capability of prime editing to perform complex DNA edits.

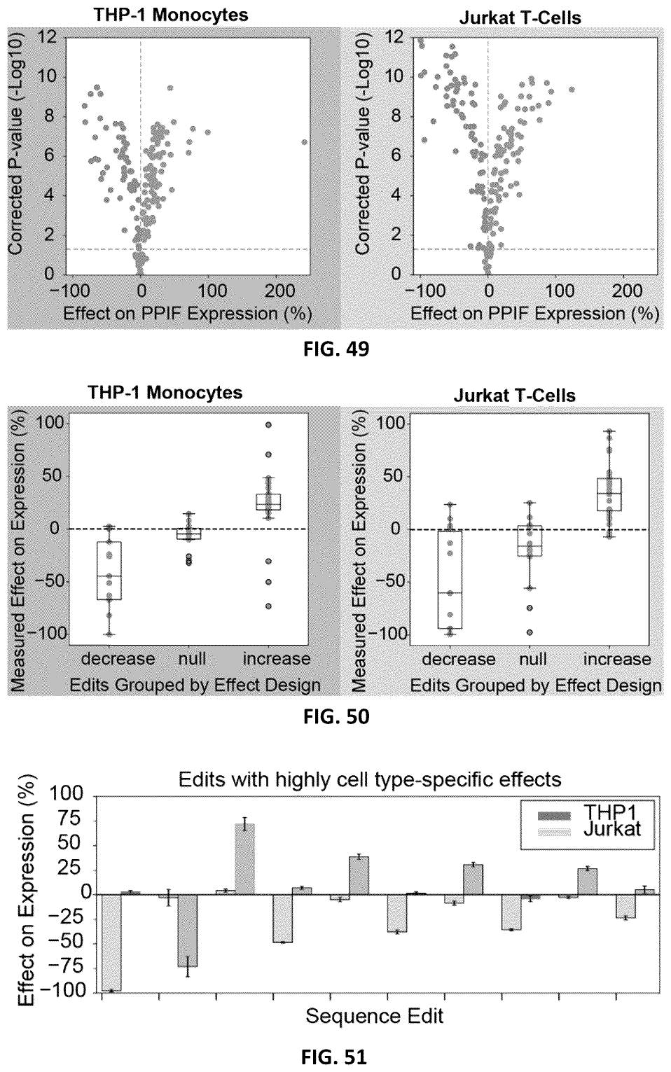

The effect sizes of the 163 edits that passed the frequency filter were highly reproducible across replicates in both cell types (Pearson R=0.99) further supporting the ability to screen hundreds of edits with Variant-FlowFISH (FIG. 48). The effects of 145 and 150 edits in this screen induced statistically significant effects in THP1 and Jurkat cells, respectively (FIG. 49). Strikingly, edits were identified that completely silenced PPIF expression (−100%) in both cell types, as well as edits that increased PPIF expression by as much as 241% (p_{adj}=1.90E-7) in THP-1 cells and 123% (p_{adj}=4.2E-10) in Jurkat cells.

Next, the alignment of the observed effect sizes with intended design goals was examined for edits optimized to achieve specific effects on expression in either cell type (FIG. 50). It was found that the average effect of each group of designed edits elicited the desired effect they were optimized for in silico. Furthermore, edits were identified in the screen that had strong effects in only one cell type (FIG. 51). These results demonstrate how the provided computational design strategy can be integrated into the provided Variant-FlowFISH workflow to improve design and testing of edits that achieve specific tuning of gene expression

Although the foregoing disclosure has been described in some detail by way of illustration and example for purpose of clarity of understanding, one of skill in the art will appreciate that certain changes and modifications within the spirit and scope of the disclosure may be practiced, e.g., within the scope of the appended claims. It should also be understood that aspects of the disclosure and portions of various recited embodiments and features can be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the disclosure. In addition, each reference provided herein is incorporated by reference in its entirety for all purposes to the same extent as if each reference was individually incorporated by reference.

What is claimed is:

1. A method of screening edited genomic sequences for their effect on expression of a target gene, the method comprising:
providing a population of cells, the genomes of the population of cells comprising a target sequence;
introducing a plurality of different target sequence edits into the genomes of the population of cells;
measuring a parameter for each cell of the population of cells, the parameter correlating with an expression level of the target gene;
separating the population of cells into a plurality of sets based on the measured parameter;
for each combination of (a) a set of the plurality of sets and (b) a target sequence edit of the plurality of different target sequence edits, determining a count of the target sequence edit incorporated into the genomes of the cells of the population of cells in the set; and
for each target sequence edit of the plurality of different target sequence edits, based on the counts in the plurality of sets, computing an effect of the target sequence edit on the expression level of the target gene.

2. The method of claim 1, wherein the plurality of different target sequence edits comprises a designed target sequence edit identified by a simulation process, the simulation process comprising:
receiving an initial target sequence edit and an initial edit site of the target sequence;
generating an initial edited sequence comprising the target sequence into which the initial target sequence edit is introduced at the initial edit site;
determining an initial predicted level of a molecular feature of a target gene in response to the initial edited sequence;
setting (i) an input target sequence edit equal to the initial target sequence edit, (ii) an input edit site equal to the initial edit site, and (iii) an input level equal to the initial predicted level of the molecular feature;
performing a series of operations in an iterative manner for a plurality of iterations, the series of operations comprising:
(a) generating a test target sequence edit and a test edit site, wherein either:
(i) the test edit site is equal to the input edit site, and the test target sequence edit is a variant of the input target sequence edit; or
(ii) the test target sequence edit is equal to the input target sequence edit, and the test edit site is one position removed from the input edit site in the target sequence;
(b) generating a test edited sequence comprising the target sequence into which the test target sequence edit is introduced at the test edit site;
(c) determining a test predicted level of the molecular feature of the target gene in response to the test edited sequence;
(d) comparing the test predicted level and the input level;
(e) based on the comparing, either
(i) updating (1) the input level to equal the test predicted level, (2) the input target sequence edit to equal the test target sequence edit, and (3) the input edit site to equal the test edit site; or
(ii) leaving the input expression level, the input target sequence edit, and the input edit site unchanged; and
subsequent to the plurality of iterations, reporting the input target sequence edit as the designed target sequence edit.

3. The method of claim 2, wherein operation (a) of the series of operations comprises
(a) generating a test target sequence edit and a test edit site, wherein either:
(i) the test edit site is equal to the input edit site; and the test target sequence edit is equal to (1) the input target sequence edit from which one nucleotide is deleted, (2) the input target sequence edit in which one nucleotide is substituted, or (3) the input target sequence edit into which one nucleotide is inserted; or
(ii) the test target sequence edit is equal to the input target sequence edit, and the test edit site is one position removed from the input edit site in the target sequence.

4. The method of claim 3, wherein:
the initial target sequence edit has a length no greater than a threshold length; and
operation (a) of the series of operations comprises
(a) generating a test target sequence edit and a test edit site, wherein either:
(i) the test edit site is equal to the input edit site; and the test target sequence edit is equal to (1) the input target sequence edit from which one nucleotide is deleted, (2) the input target sequence edit in which one nucleotide is substituted, or, if the input target sequence edit has a length at least one bp shorter than the threshold length, (3) the input target sequence edit into which one nucleotide is inserted; or
(ii) the test target sequence edit is equal to the input target sequence edit, and the test edit site is one position removed from the input target sequence edit in the target sequence.

5. The method of claim 4, wherein the threshold length is no greater than 35 bp.

6. The method of claim 2, wherein the designed target sequence edit is an insertion sequence.

7. The method of claim 2, wherein the molecular feature of the target gene comprises a gene expression level, a binding of a transcription factor, an epigenetic mark, or an accessibility to a chromatin.

8. The method of claim 7, wherein the molecular feature of the target gene is an expression level of the target gene operably linked to the target sequence.

9. The method of claim 7, wherein the determining of the initial predicted level and the test predicted level comprises use of Cap Analysis of Gene Expression (CAGE).

10. The method of claim 2, wherein the receiving of the initial target sequence edit comprising randomly generating the initial target sequence edit.

11. The method of claim 2, wherein the plurality of iterations consists of at least 10 iterations.

12. The method of claim 2, wherein the method further comprises performing the simulation process, thereby identifying the designed target sequence edit.

13. The method of claim 1, wherein:
the population of cells contains a fusion protein, the fusion protein comprising a polymerase and a DNA targeting protein, the DNA targeting protein having a nickase activity and binding to the target sequence;
the introducing of the plurality of different target sequence edits comprises introducing to the population of cells a plurality of template polynucleotides, the template polynucleotides each independently comprising a target sequence edit of the plurality of different target sequence edits; and
in at least a portion of the population of cells, a template polynucleotide of the plurality of template polynucleotides is primed by a target sequence nicked by the DNA targeting protein, and copied into the genome by the polymerase.

14. The method of claim 13, wherein a plurality of prime-editing guide RNA (pegRNA) molecules comprises the plurality of template polynucleotides.

15. The method of claim 14, wherein the plurality of pegRNA molecules further comprises a gRNA spacer no more than 50 bp away from the target sequence edit.

16. The method of claim 14, wherein the plurality of pegRNA molecules further comprises a scaffold sequence having at least 85% sequence identity with the sequence:
GTTTAAGAGCTATGCTGGAAACAGCATAGCAAGTTTAAATAAGGCTAGTC
CGTTATCAACTTGAAAAAGTGGCACCGAGTCGGTGC.

17. The method of claim 14, wherein the plurality of pegRNA molecules further comprises a primer-binding site (PBS) having a length between 6 bp and 16 bp.

18. The method of claim 14, wherein the plurality of pegRNA molecules further comprises a reverse transcription (RT) template having a length no greater than 60 bp.

19. The method of claim 18, wherein the reverse transcription (RT) template extends between 6 bp and 16 bp upstream of the target sequence edit.

20. The method of claim 14, wherein the plurality of pegRNA molecules further comprises a 3′ end modification having at least 86% sequence identity with the 37-bp trimmed evopreRQ (tevopreQ1) pseudoknot sequence.

21. The method of claim 13, wherein the introducing of the plurality of template polynucleotides comprises use of a lentivirus, a transposase, or a recombinase.

22. The method of claim 21, wherein the introducing of the plurality of template polynucleotides comprises transducing the population of cells with lentivirus.

23. The method of claim 13, wherein the method further comprises introducing to the population of cells an inducer of expression of the fusion protein.

24. The method of claim 23, wherein the population of cells comprises cells of an inducible Cas9 RT-nickase cell line.

25. The method of claim 1, wherein the determining of the counts of the plurality of different target sequence edits comprises:
receiving a plurality of sequence reads;
for each of the plurality of sequence reads, determining if the sequence read belongs to an aligning set consisting of the sequence reads of the plurality of sequence reads that align to a reference amplicon, the reference amplicon comprising the target sequence;
for each variant sequence of a set of variant sequences, determining a count of the sequence reads of the aligning set that comprise the variant sequence, each variant sequence of the set of variant sequences independently comprising the target sequence modified according to a target sequence edit of the plurality of different target sequence edits; and
determining a count of the sequence reads of the aligning set that do not match any variant sequence of the set of variant sequences, and that differ from the target sequence by no more than a threshold percentage of base pairs.

26. The method of claim 25, wherein the threshold percentage is no greater than 15%.

27. The method of claim 1, wherein the computing of the effect of the target sequence edit comprises:
for each target sequence edit of the plurality of different target sequence edits, fitting the counts in the plurality of sets to a log-normal distribution; and
calculating a percent difference between (a) the log-normal distribution for the target sequence edit, and (b) a log-normal distribution derived by fitting counts of the target sequence in the plurality of sets.

28. The method of claim 1, wherein the target sequence comprises a cis-regulatory element (CRE) operably linked to the target gene.

29. The method of claim 1, wherein the plurality of different target sequence edits each independently comprise one or more substitutions, insertions, deletions, or a combination thereof, relative to the target sequence.

30. The method of claim 1, wherein the determining of the count of the plurality of different target sequence edits comprises sequencing a target region of the genomes of the population of cells, the target region comprising the target sequence prior to the introducing of the plurality of different target sequence edits into the population of cells.

31. The method of claim 1, wherein the plurality of different target sequence edits comprises at least 3 different target sequence edits.

32. The method of claim 1, wherein the plurality of sets comprises at least 3 sets.

33. The method of claim 1, wherein the measuring of the parameter comprises using a phenotypic assay.

34. The method of claim 33, wherein the phenotypic assay comprises a fluorescence-in-situ-hybridization (FISH) assay, a fluorescence assay, an immunofluorescence assay, a growth assay, or a combination thereof.

35. The method of claim 1, wherein the separating of the population of cells comprises fluorescence activated cell sorting (FACS).

36. A composition comprising the plurality of template polynucleotides of claim 13.

37. A population of cells comprising the plurality of template polynucleotides of claim 13.

38. A cell comprising a genome incorporating a target sequence edit of the plurality of different target sequence edits of claim 1.

39. The cell of claim 38, wherein the cell is a monocyte or T cell.

40. A method of treating a subject having a disease dependent on expression of the target gene of claim 13, the method comprising administering to the subject a therapeutically effective amount of the plurality of template polynucleotides of claim 13.

41. The method of claim 40, wherein the target gene encodes peptidylprolyl isomerase F (PPIF).

42. The method of claim 41, wherein the disease comprises inflammatory bowel disease.

43. A computer product comprising a non-transitory computer readable medium storing a plurality of instructions that, when executed, cause a computer system to perform the method of any one of claims 1-35.

44. A system comprising:
the computer product of claim 43; and
one or more processors for executing instructions stored on the computer readable medium.
